(19) United States
(12) Patent Application Publication
(10) Pub. No.: US 2021/0260101 A1
(43) Pub. Date: Aug. 26, 2021

(54) POTENTIATED ANTIBIOTIC COMPOSITIONS AND METHODS OF USE FOR TREATING BACTERIAL INFECTIONS AND BIOFILMS

(71) Applicant: The Board of Regents of the University of Oklahoma, Norman, OK (US)

(72) Inventors: Charles V. Rice, Norman, OK (US); Daniel T. Glatzhofer, Norman, OK (US)

(21) Appl. No.: 17/231,854

(22) Filed: Apr. 15, 2021

Related U.S. Application Data
(63) Continuation-in-part of application No. PCT/US2019/054508, filed on Oct. 3, 2019.
(60) Provisional application No. 62/747,517, filed on Oct. 18, 2018.

(51) Int. Cl. A61K 31/785; A61K 31/407; A61K 31/427; A01N 43/44; A01N 43/86; A01N 33/04; A61P 31/04

(52) U.S. Cl. CPC A61K 31/785; A61K 31/407; A61K 31/427; A61P 31/04; A01N 43/86; A01N 33/04; A01N 43/44

(57) ABSTRACT

Compositions of β-lactam antibiotics and branched poly(ethylenimine) (BPEI), and β-lactam antibiotics and potentiating compounds of polyethylene glycol (PEG)-BPEI conjugates, and methods of their use to treat infections and to remove bacterial biofilms from surfaces of devices and wounds. The BPEI and PEG-BPEI conjugates potentiate the activity of the β-lactam antibiotics so the compositions have synergistic effects against various Gram-positive bacteria. For example, the compositions can be used to treat Gram-positive bacteria, such as Methicillin-resistant Staphylococcus aureus (MRSA) and Methicillin-resistant Staphylococcus epidermidis (MRSE), that have developed resistance against most β-lactam antibiotics. The BPEI and PEG-BPEI conjugates result in the resensitization of such resistant bacterial strains to traditional antibiotic therapies such as β-lactam antibiotics.

### CROSS REFERENCE TO RELATED APPLICATIONS

This application is a continuation-in-part national stage filing of PCT Application No. PCT/US2019/054508, filed Oct. 3, 2019, which claims benefit under 35 USC § 119(e) of Provisional Application U.S. Ser. No. 62/747,517, filed Oct. 18, 2018. This application also claims the benefit of U.S. Ser. No. 17/208,176, filed Mar. 22, 2021, which is a continuation-in-part application of U.S. Ser. No. 16/530,756, filed Aug. 2, 2019, which is a continuation application of U.S. Ser. No. 15/736,675, filed Dec. 14, 2017, which is a national stage filing of PCT Application No. PCT/US2016/037799, filed Jun. 16, 2016, which claims the benefit of U.S. Provisional Application Ser. No. 62/180,976, filed Jun. 17, 2015. The entire contents of each of the applications listed above is hereby expressly incorporated herein by reference.

### STATEMENT REGARDING FEDERALLY SPONSORED RESEARCH OR DEVELOPMENT

This invention was made with government support from National Institutes of Health (NIH) National Institute of General Medical Sciences (NIGMS) grant 1R01GM090064-01, NIH grant R03AI142420-01, and National Science Foundation grant 1352604). The government has certain rights in the invention.

### BACKGROUND

Resistance of certain bacterial strains to antibiotics which were previously-effective against the strains is a growing global problem. For example, colonies of methicillin-resistant Staphylococcus aureus (MRSA) bacteria invade host tissue to release toxins that cause tissue injury, leading to significant patient morbidity. The patient suffers while numerous first- and second-line antibiotics are prescribed to no avail. This increases the threat of MRSA to public health. Timely MRSA diagnosis and delivering drugs of last resort are essential to prevent mortality. In 2011 for example, MRSA infected 80,500 people and nearly 1 in 7 cases resulted in death (11,300; 14%). While, several antibiotics of last resort (vancomycin, linezolid, daptomycin) are effective at killing MRSA, and there has never been a S. aureus isolate resistant to all approved antibiotics, patients still die from MRSA infections. The reason for this is because the drugs of last resort are given after morbidity from staphylococcal toxins has set in, too late to prevent mortality. Moreover, vancomycin, a primary treatment option after MRSA diagnosis, presents additional barriers of high cost and toxicity. New antibiotics, such as oxadiazoles, tedizolid, and teixobactin, are awaiting FDA approval to meet the critical need for new treatments because S. aureus strains resistant to vancomycin and β-lactams have emerged. New treatment options for MRSA and other bacterial strains which have become resistant to standard β-lactam antibiotics are needed.

Staphylococci, especially Staphylococcus epidermidis and Staphylococcus aureus, present serious hardships to clinical infectious disease management of biofilms within inner surfaces of implanted medical devices (e.g., catheters). Hundreds of millions of intravascular devices are used annually and the cost of infection resulting from their use is $1-3 billion annually. The current clinical practice guidelines for managing these infections includes replacing the device and/or creating a bolus of high antibiotic concentration inside the catheter lumen (antibiotic lock therapy, or ALT). It is estimated that ALT requires 100-1000 times higher concentrations of antibiotics than normal to kill bacteria within biofilms The danger from biofilms is amplified by antimicrobial resistance. A 2004 survey found that nearly 90% of clinical isolates S. epidermidis had oxacillin resistance. Vancomycin (at 1000×MIC) is the recommended antibiotic for treating drug resistant biofilms while linezolid is held in reserve. Improved outcomes and lower medical costs would result by overcoming biofilm barriers created by the matrix of extracellular polymeric substances (EPS).

Antimicrobial resistance (AMR) is a critical and increasing world threat to health. The CDC estimates that at least 2 million people in the U.S. alone are infected by antibiotic-resistant bacteria annually, leading to 23,000 deaths and $20 billion/year in US healthcare costs. The danger from AMR is amplified by microbial biofilms, whose EPS are physical barriers against antimicrobial agents. Biofilms from staphylococci are predominantly caused by S. epidermidis and S. aureus. MRSA has become a serious threat to public health because, in addition to drug resistance, it releases virulence factors and potent toxins. In contrast, the threat from methicillin-resistant S. epidermidis (MRSE) appears lower because of reduced virulence and fewer toxins. However, with its ubiquitous niche on the human skin, the seriousness of MRSE cannot be overlooked. S. epidermidis bacteria can be found on nearly every medical device. The combination of biofilm formation and AMR create defense mechanisms enabling MRSE to become a leading cause of chronic infections. Coagulase-negative staphylococci (CoNS), such as S. epidermidis, cause more infections associated with central arterial lines than coagulase-positive S. aureus.

Staphylococcus epidermidis belongs to the Gram-positive Staphylococcus genus. It has emerged as one of the most common causes of healthcare-associated infections due to the increasing use of medical implant devices. Unlike the coagulase-positive Staphylococcus aureus, S. epidermidis does not produce coagulase and therefore is classified as CoNS. Accounting for about 70% of all CoNS on human skin, S. epidermidis is a leading cause of severe bloodstream infections. Approximately 80,000 cases of central venous catheter infections per year in the US are caused by S. epidermidis. Most of the CoNS lack aggressive virulence factors (like those in S. aureus) and instead owe their pathogenic success to the ability to form biofilms.

Biofilms are multicellular agglomerations of microorganisms enclosed in a matrix of EPS. Containing polysaccharides, proteins, and extracellular DNA, the EPS matrix acts as a shield that protects the organisms from host defenses and antibiotics. Biofilms can adhere to either biotic or abiotic surfaces—such as cardiac pacemakers and catheters—and have a highly regulated defense mechanism that grants intrinsic resistance against antimicrobial agents. Biofilm development starts with an initial attachment of planktonic cells to a surface, which then grow into clusters of multicellular colonies. Subsequent cell-cell adhesions, divisions, and secretion of EPS create a three-dimensional architecture designed to channel water and supply nutrients to the inner layers, thereby allowing for biofilm maturation. While the outer-layer cells remain metabolically active, the inner-layer cells are persister bacteria that often stay in a dormant state, and thus are the most difficult to eradicate with antimicrobial treatments, that only target growing organisms. During biofilm maturation, part of the biofilm can detach and disperse planktonic cells, which spread to colonize new surfaces. Mechanisms of biofilm maturation and detachment are poorly understood, but studies suggest that dispersed cells are more virulent and heighten the risk of acute infections.

Biofilm defense mechanisms reduce antibiotic efficacy. The antibiotic concentrations required to eradicate biofilms are ten-fold to a thousand-fold higher than the concentrations required to kill bacteria in planktonic form, creating a burden on both public health and the economy from increased medical costs. Removal of biofilm-infected indwelling medical devices complicates treatments and interferes with the healing process. Additionally, persister biofilms are also a leading cause of chronic wound infections and poor wound healing. Around 90% of chronic wound specimens—compared to only 6% for acute wounds—were found to contain biofilms in which the prevalent species was Staphylococci. Thus, few publications offer information on S. epidermidis biofilm properties and antibiofilm testing, and the virulence and resistance factors of S. epidermidis biofilms are poorly understood. There is thus a great and expanding need to develop treatments for these dangerous biofilm infections.

The prognosis is worse for wounds with biofilms of AMR bacteria, such as MRSA, MRSE, and multi-drug resistant Pseudomonas aeruginosa (MDR-PA). Resistance hinders initial treatment of standard of care antibiotics. The persistence of MRSA, MRSE, and/or MDR-PA often allows acute infections to become chronic wound infections.

MRSA and MRSE are dangers in nosocomial environments where 90% of all hospital patients receive an I.V. device and 13% receive a peripherally inserted central catheter (“PICC line”). Between 2011 and 2014, their associated infections (central line associated bloodstream infections, CLABSI) were 37% hospital-acquired device-associated infections. Within CLABSIs, 16.4% are caused by coagulase-negative staphylococci. S. epidermidis is the predominant CoNS species and 13.2% are caused by S. aureus. Adjuvants to improve antimicrobial efficacy target either biofilms or MRSE/MRSA.

A therapeutic compound able to address both the physical barrier inherent in biofilms and the genetic barriers of AMR and would be a highly desired tool in treating the increasingly dangerous array of bacterial infections facing the world today.

### BRIEF DESCRIPTION OF THE DRAWINGS

Several embodiments of the present disclosure are hereby illustrated in the appended drawings. It is to be noted however, that the appended drawings only illustrate several embodiments and are therefore not intended to be considered limiting of the scope of the present disclosure.

FIG. 1 is a schematic of a representative model branched polyethylenimine (BPEI) polyamine containing 1°, 2°, and 3° amines reacting with a 1000 Da polyethylene glycol (PEG₁₀₀₀) molecule having a glycidyl epoxide end-group. Preference for reacting at 1° amines rather than 2° amines is governed by temperature.

FIG. 2 shows ¹H NMR spectra of 600-Da BPEI (BPEI₆₀₀) (A), 1000-MW PEG-epoxide (B), and their reaction product (C). The lack of epoxide signals in (C) indicates the reaction is complete.

FIG. 3 shows a library of BPEI₆₀₀ compounds with 1° amines reacting with an ethyl, diglyme, or PEG molecules having a glycidyl epoxide end-group. The capping groups are hydrophilic but vary in steric bulk.

FIG. 4 shows BPEI compounds formed by 1° amine capping reactions with ethyl, diglyme, or PEG molecules.

FIG. 5 shows a general reaction mechanism for forming an anhydride of BPEI for use in the embodiments of the present disclosure.

FIG. 6 shows a general reaction for forming BPEI-acetic anhydride for use in the embodiments of the present disclosure.

FIG. 7 shows a general reaction for forming BPEI-propionic anhydride for use in the embodiments of the present disclosure.

FIG. 8 shows a general reaction mechanism for forming an acrylamide of BPEI for use in the embodiments of the present disclosure.

FIG. 9 shows a general reaction for forming BPEI-acrylamide for use in the embodiments of the present disclosure.

FIG. 10 shows a general reaction for forming BPEI-isopropylacrylamide for use in the embodiments of the present disclosure.

FIG. 11 shows a general reaction for forming BPEI-methyl 2-(trifluoroethyl)acrylate for use in the embodiments of the present disclosure.

FIG. 12 shows a general reaction for forming BPEI-ethylene glycol dimethacrylate for use in the embodiments of the present disclosure.

FIG. 13 shows a general reaction for forming BPEI-isocyanatoethylmethacrylate for use in the embodiments of the present disclosure.

FIG. 14 shows a general reaction for forming BPEI-methyl methacrylate for use in the embodiments of the present disclosure.

FIG. 15 shows a general reaction for forming BPEI-N-2-hydroxypropyl methacrylamide for use in the embodiments of the present disclosure.

FIG. 16 shows a general reaction for forming BPEI-methylenebisacrylamide for use in the embodiments of the present disclosure.

FIG. 17 is a schematic representation of the experimental procedure of a microtiter biofilm model for synergistic effect screening against MRSE biofilms. MBEC assays were carried out using MBEC inoculator, which is a microtiter plate lid with protruding prongs attached. Each prong fits into each well and allows bacterial biofilm to form and grow.

FIG. 18 shows Scanning Electron Micrographs of the tip of MBEC prongs. A control prong with no bacteria is shown (A). MRSE 35984 biofilm colonies were formed after 24 hours of inoculation (B); the arrows highlight some of the biofilm microcolonies. Scale bars=200 μm.

FIG. 19 shows Scanning electron micrographs of a MRSE 35984 biofilm. The intercellular matrices of EPS are captured as they wrap around every bacterium (A). At higher magnification, the EPS matrix is clearly shown to be sheltering the whole bacterial colony in an amorphous coat (B). Scale bars=2 μm.

FIG. 20 shows the synergistic effects of BPEI and antibiotics against MRSE 35984 (A) and MRSE 29887 (B) on a 96-well checkerboard pattern. The synergy was seen both on the planktonic challenge plates (Aa and Ba) and the biofilm MBEC assays (Ab and Bb).

FIG. 21 shows mature MRSE 35984 biofilms stained with crystal violet treated with 600-Da BPEI for 20 hours, as well as the negative and positive controls. The dissolved biofilm solutions were transferred to a new plate, and the biofilm remainders are shown as top-down view, (A). The mean OD₅₅₀ of the dissolved biofilms was measured, (B). Error bars denote standard deviation (n=10). The MRSE biofilms were significantly dissolved by BPEI₆₀₀ (t-Test, p-value<0.01, significant difference between the negative control and each treatment is indicated with an asterisk).

FIG. 22 shows mature MRSE 35984 biofilms stained with crystal violet were treated with 10,000-Da BPEI (BPEI_{10,000}) for 20 hours, as well as the negative and positive controls. The dissolved biofilm solutions were transferred to a new plate, and the biofilm remainders are shown as top-down view, (A). The mean OD₅₅₀ of the dissolved biofilms was measured, (B). Error bars denote standard deviation (n=10). The MRSE biofilms were significantly dissolved by BPEI_{10,000} (t-Test, p-value<0.01, significant difference between the negative control and each treatment is indicated with an asterisk).

FIG. 23 shows crystal violet absorbance representing MRSE 35984 biofilm biomass. Strong antibiofilm formation synergy between BPEI and piperacillin was observed, compared to individual piperacillin or BPEI treated samples. Error bars denote standard deviation (n=3). PIP, piperacillin.

FIG. 24 shows biofilm kill curves of MRSE 35984 by various treatments. Only the combination treatment of BPEI+oxacillin (64 μg/mL+16 μg/mL)—the diamond-curve—could eradicate MRSE 35984 biofilms. Error bars denote standard deviation (n=2). CFU, colonies forming units.

FIG. 25 shows Scanning electron micrographs of mature MRSE 35984 biofilms (3-day old). The untreated control sample shows thick EPS enfolding every bacterial cell (A). BPEI-treated sample shows disrupted EPS and significant number of exposed cells without the EPS (B). At lower magnification, the untreated control (C) biofilms appear with full and tightly occupied biofilms, while the BPEI-treated sample (D) shows disjointed biofilms by many revealed surfaces. Scale bars (A and B)=1 μm. Scale bars (C and D)=100 μm.

FIG. 26 shows checkerboard assays showing reduction of oxacillin MIC in MRSE 35984 when treated with BPEI₆₀₀ (Upper Panel) or PEG₃₅₀-BPEI₆₀₀ (Lower Panel). Each assay was performed in triplicate and presented as the average change in optical density at 600 nm (OD₆₀₀).

FIG. 27 shows checkerboard assays showing reduction of oxacillin MIC in MRSA USA300 when treated with BPEI₆₀₀ (Upper Panel) or PEG₃₅₀-BPEI₆₀₀ (Lower Panel). Each assay was performed in triplicate and presented as the average change in optical density at 600 nm (OD₆₀₀).

FIG. 28 shows checkerboard assays showing reduction of oxacillin MIC in MRSA MW2 when treated with BPEI₆₀₀ (Upper Panel) or PEG₃₅₀-BPEI₆₀₀ (Lower Panel). Each assay was performed in triplicate and presented as the average change in optical density at 600 nm (OD₆₀₀).

FIG. 29A demonstrates the efficacy of BPEI₆₀₀+piperacillin against P. aeruginosa 27853. P. aeruginosa 27853 is susceptible to piperacillin (MIC≤16 μg/mL) however BPEI₆₀₀ improves piperacillin efficacy. Assay was performed in triplicate and presented as the average change in OD₆₀₀.

FIG. 29B demonstrates the efficacy of PEG₃₅₀-BPEI₆₀₀+piperacillin against P. aeruginosa 27853. PEG₃₅₀-BPEI₆₀₀ improves piperacillin efficacy. Assay was performed in triplicate and presented as the average change in OD₆₀₀.

FIG. 29C demonstrates the efficacy of BPEI₆₀₀+piperacillin against the clinical isolate P. aeruginosa OU1 (PA OU1), which is resistant to piperacillin. Resistance to piperacillin is overcome in the presence of BPEI₆₀₀. Assay was performed in triplicate and presented as the average change in OD₆₀₀.

FIG. 29D demonstrates the efficacy of PEG₃₅₀-BPEI₆₀₀+piperacillin against PA OU1. Resistance to piperacillin is overcome in the presence of PEG₃₅₀-BPEI₆₀₀. Assay was performed in triplicate and presented as the average change in OD₆₀₀.

FIG. 30A shows isothermal titration calorimetry data that demonstrate that P. aeruginosa LPS binds with BPEI₆₀₀.

FIG. 30B shows isothermal titration calorimetry data that demonstrate that P. aeruginosa LPS binds with PEG₃₅₀-BPEI₆₀₀.

FIG. 30C is a graphic which illustrates how PEGylation of BPEI₆₀₀ could reduce piperacillin potentiation. The differences in binding energetics and molar ratio between BPEI₆₀₀ and PEG₃₅₀-BPEI₆₀₀ may be attributed to steric hinderance from the PEG₃₅₀ group attached to BPEI₆₀₀.

FIG. 31 is a photograph of nitrocefin β-lactam ring hydrolysis assay results. Various amounts of BPEI₆₀₀ or PEG₃₅₀-BPEI₆₀₀ were distributed in the wells of a 96-well plate. Each well contained 0.005 moles of nitrocefin. After 30 minutes, hydrolysis caused the yellow color of nitrocefin to become red. These data indicate that nitrocefin is 100-times less susceptible to hydrolysis from PEG₃₅₀-BPEI₆₀₀ as compared to the unmodified BPEI₆₀₀.

FIG. 32A shows photographs of the results of biofilm disruption assays using crystal violet to stain the biomass. Preformed MRSE 35984 biofilms were stained with crystal violet and washed prior to treatment with different concentrations of PEG₃₅₀-BPEI₆₀₀ or BPEI₆₀₀, in addition to treatment with of water only and acetic acid. The stained biomass dissolved by the test agent was transferred into a new plate (lower panel). The biomass remaining in the original plate are shown in the upper panel.

FIG. 32B shows the absorbance of the dissolved biomass of FIG. 32A at 550 nm. Error bars denote standard deviation (n=6). An asterisk indicates a significant difference between the treatments and the negative control of water (t-test, p-value<0.01).

FIG. 33A shows the synergy between BPEI₆₀₀ and ampicillin against MRSA 43300. Checkerboard assay data on planktonic bacteria are shown on the left (i) and corresponding biofilm data are shown on the right (ii).

FIG. 33B shows the synergy between BPEI₆₀₀ and ampicillin against clinical isolate MRSA OU6. Checkerboard assay data on planktonic bacteria are shown on the left (i) and corresponding biofilm data are shown on the right (ii).

FIG. 33C show the synergy between BPEI₆₀₀ and ampicillin against clinical isolate MRSA OU11. Checkerboard assay data on planktonic bacteria are shown on the left (i) and corresponding biofilm data are shown on the right (ii).

FIG. 34 shows in upper panel A results of MRSA OU6 biofilms stained with crystal violet and treated with polymyxin B (PmB) and BPEI₆₀₀ for 20 hours, as well as a negative control (water only) and a positive control (30% acetic acid). The dissolved biofilm solutions were transferred to a new plate, and the biofilm remainders are shown as top-down view. The mean OD₅₅₀ of the dissolved biofilm solution was measured, results shown in lower panel B. Error bars denote standard deviation (n=10).

FIG. 35 shows SEM images of MRSA OU11 biofilms on glass coverslips. Untreated control biofilms are shown to be covered and wrapped around in the matrix of EPS (A and C). BPEI₆₀₀-treated samples have much less EPS with many cells being exposed (B and D). Scale bars in A and B=2 μm. Scale bars in C and D=1 μm.

FIG. 36 shows SEM images of established MRSA OU6 biofilms on PC membranes. A very thick coating of the EPS matrix is present in the untreated control biofilm on the PC membrane which also blocks the bacterial cells from being captured in the microscope (A). BPEI₆₀₀-treated sample has a much clearer view as the EPS removed and even the membrane surface is exposed as many nano-size pores are seen at the bottom (B). Scale bars=1 μm.

FIG. 37 is an illustration of the P. aeruginosa outer membrane in which metal ions stabilize the LPS O-antigen, outer-core, inner-core, and lipid A moieties. This presents a barrier to the passive diffusion of β-lactam antibiotic to porin transports. Many other compounds, such as erythromycin, rely on passive diffusion to reach the periplasm.

FIG. 38 is a checkerboard assay data demonstrating that sub-lethal amounts of 600-Da BPEI lower the piperacillin MIC against PA OU1, that exhibits multidrug resistance against aztreonam, cefepime, ceftazidime, ciprofloxacin, meropenem and piperacillin/tazobactam. The MIC of piperacillin (64 μg/mL) is resistant but 2 μg/mL of BPEI₆₀₀ (3.3 μM) reduces the β-lactam MIC to 4 μg/mL which is interpreted as susceptibility.

FIG. 39 shows growth curves of PA BAA-47 shows that sub-lethal amounts of BPEI₆₀₀ and piperacillin slow bacterial growth but do not kill the culture. Treating the culture with a combination of BPEI₆₀₀ and piperacillin, each at sub-lethal concentrations, stops growth. Error bars denote standard deviation (n=2) and, for some data points, are smaller than the data symbol.

FIG. 40 shows raw ITC data of (A) of BPEI₆₀₀ interacting with P. aeruginosa LPS. BPEI₆₀₀ (0.64 mg/mL) was titrated into LPS (5 mg/mL) via 2 μL injections in 50 mM Tris-HCl (pH 7) buffer at 25° C. The raw data in (A) indicate an exothermic binding event which can be quantified by conversion to an ITC thermogram (B). The thermogram abscissa is generated from the molar ratio of each species. Here, the molecular mass of LPS was estimated to be 20 kDa.

FIG. 41 is an illustration of how BPEI₆₀₀ binds to LPS and facilitate the passive diffusion of β-lactams toward porin transporters. Higher concentrations are required to increase the uptake of non-β-lactams (such as erythromycin), and at the highest concentration, BPEI₆₀₀ exhibits its own antibacterial properties.

FIG. 42 shows the effect of BPEI₆₀₀ on the intracellular accumulation of the DNA-binding fluorescent probe H33342 in a P. aeruginosa PAO1 strain with drug resistance. Real-time kinetics of H33342 uptake show that BPEI₆₀₀ significantly increased the H33342 accumulation (closed circles) into the bacterial cells, compared to the untreated control (open circles). Similar effects are seen with the efflux deficient mutant PaΔ3 (open and closed diamonds). The intracellular concentration of H33342 in the treated cells is higher than the wild-type cells indicating that BPEI₆₀₀ does not hinder efflux processes. Error bars denote standard deviation (n=5).

FIG. 43A shows the concentration dependence of influx of H33342 into wild-type PAO1 cells. Competition between influx and efflux processes in the viable cells results in overlapping data points.

FIG. 43B shows the concentration dependence of influx of H33342 into an efflux-deficient mutant strain of PAO1. In this efflux-deficient mutant strain, higher concentrations of BPEI₆₀₀ resulted in higher dye concentrations. (n=5)

FIG. 44 shows that the uptake of H33342 is a multi-step process with exponential kinetics. This phenomenon can be identified by plotting the natural logarithm of dye concentration versus time. The increasing slope with concentration shows that the rate of influx increases as the passive diffusion barriers are lowered from BPEI₆₀₀ binding to additional anionic sites on LPS molecules.

FIG. 45 shows results that indicate BPEI binds to LPS through anionic sites on the inner-core, outer-core, and O-antigen regions. These sites also bind divalent metal ions. The growth media contains trace amounts of metal ions and thus the anionic regions of LPS are not fully occupied. This provides an opportunity for BPEI₆₀₀ to bind with LPS and increase H33342 influx (triangles) compared to untreated cells (open circles). Addition of 2 mM MgCl₂ to BPEI-treated cells results in a reduction of dye influx (open squares) as the metals ions occupy remaining anionic sites and restore LPS barriers to diffusion. When metal ions are added first (closed squares), all LPS anionic sites are occupied, preventing the binding of BPEI₆₀₀ that would otherwise increase dye influx. n=5.

FIG. 46 demonstrates that the dye 1-N-phenylnaphthylamine (NPN) accumulates in hydrophobic regions and fluoresces when bound to phosphate groups. Polymyxin-B (PmB) allows greater uptake of NPN than BPEI₆₀₀. The sub-lethal concentrations of BPEI₆₀₀ allow NPN access to the membrane without affecting cell viability. (n=5)

FIG. 47 shows scanning electron micrograph images of treated PA BAA-47 cells. Untreated control cells appear with regular rod-shape of about 2-3 μm long (A). BPEI₆₀₀ treated cells (4 μg/mL) (B) and piperacillin treated cells (1 μg/mL) (C) show inconsistency in their size with longer lengths but the rod-shape remains. Combination of 4 μg/mL+1 μg/mL piperacillin treated cells (D) show extreme distortions both in size and shape with insets (E) and (F) for higher magnifications. Scale bars=2 μm.

FIG. 48 shows biofilm eradication assay data using collected with the Calgary biofilm device. EPS creates additional barriers to piperacillin efficacy and thus 256 μg/mL are required to kill the bacteria. However, BPEI₆₀₀ disrupts the biofilm EPS and increases β-lactam access to the cells, reducing the MBEC to 8 μg/mL.

FIG. 49 is an illustration of BPEI₆₀₀ bind to and dispersing the exopolymeric substances (EPS) of P. aeruginosa BAA-47 bacteria. The dispersal of EPS allows antibiotics, such as piperacillin (PIP) to reach the bacteria and kill them. The presence of BPEI₆₀₀ also enables reduction of the LPS diffusion barrier to potentiate antibiotic efficacy.

### DETAILED DESCRIPTION

The present disclosure is directed to potentiated antibiotic compositions and their use in treating bacterial infections and biofilms. In at least certain embodiments, the present disclosure is directed to novel compositions comprising antibiotics against which certain bacteria (e.g., MRSA) strains have become resistant. In other words, the bacterial strains have become resensitized to the novel antibiotic formulations of the present disclosure which comprise historical antibiotics, such as, but not limited to, the β-lactams, for example, methicillin, amoxicillin, and ampicillin, and others described elsewhere herein. In particular, results provided herein show that the lost anti-MRSA effectiveness of certain FDA-approved antibiotics, such as ampicillin (or other antibiotic listed elsewhere herein), can be restored via a synergistic effect when they are administered conjointly with BPEI, a cationic polyamine. Further, the effective levels (i.e., the minimum inhibitory concentration (MIC)) of certain other antibiotics can be substantially reduced (e.g., by about ten-fold) when administered with BPEI.

The compositions of the present disclosure also include, but are not limited to, β-lactam antibiotics used conjointly with a BPEI, such as a low molecular-weight BPEI (e.g. BPEI₆₀₀), to which is conjugated a polyethylene glycol (PEG) molecule to form a PEG-BPEI compound (also referred to herein as a PEGylated BPEI). As discovered herein, in non-limiting embodiments, β-lactam antibiotics that kill methicillin-susceptible S. aureus (MSSA)) are also able to prevent and/or reduce the growth of MRSA when administered with PEG-BPEI con jugate. The β-lactam+BPEI and β-lactam+PEG-BPEI combinations of the disclosure are also effective against exopolymers (the EPS matrix) that surround MRSE bacteria and other bacteria. The BPEI compounds can also potentiate antibiotics, such as oxacillin, vancomycin, rifampin and linezolid, to improve their efficacy against biofilms comprising resistant bacteria. BPEI has been found to disable β-lactam antibiotic resistance from penicillin binding protein 2a (PBP2a). PEG-BPEIs of the present disclosure can potentiate antibiotics against drug-resistant and drug-susceptible forms of S. epidermidis (MRSE and MSSE, respectively) and drug-resistant and drug-susceptible forms of S. aureus (MRSA and MSSA, respectively) when these pathogens are planktonic (free-living) or sequestered in biofilms. Thus, the PEG-BPEI+antibiotic compositions and combinations described herein function kill both bacterial pathogens in isolation and in the biofilms that contain these pathogens. For example, in certain embodiments, the PEG-BPEI+antibiotic compositions and combinations described herein can be used to kill or inhibit the growth of a microbial biofilm on a tissue surface of a subject, such as an epithelial or endothelial lining of an organ or vessel within the body of a patient or on a surface of an external or internally implanted medical device.

In certain embodiments, the compositions of the present disclosures may be applied topically to an external or internal wound to treat a planktonic or biofilm bacterial infection in or on the wound. The treated wounds may be acute or chronic. Acute wounds are typically due to some type of trauma and include, for example, abrasions, lacerations, punctures, avulsions and incisions. Chronic or “non-healing” wounds include wounds such as diabetic foot ulcers, venous leg ulcers, pressure ulcers (e.g., bed sores), wounds due to arterial insufficiency, radiation wounds, and non-healing surgical wounds (e.g., due to abdominal surgery). Evidence indicates that bacterial biofilms play a significant role in the inability of chronic wounds to heal properly, since biofilms are present in only about 6% of acute wounds but are present in about 90% of chronic wounds. The biofilm apparently impairs or interferes with the normal growth factors and other endogenous chemicals necessary for the growth of epithelial tissues. Debridement of the wound can remove some of the biofilm but cannot be 100% effective. The compositions of the present disclosure can be much more effective in attacking the biofilms than just their physical removal.

Without wishing to be bound by theory, it is believed that the BPEIs target wall teichoic acid (WTA), an essential cofactor for PBP2a and PBP4 function and also an essential component of biofilms. These compounds depart from the status quo drug activity of stopping WTA biosynthesis in the cytoplasm and instead target mature WTA in the cell wall and WTA within the biofilm matrix. In certain embodiments, the PEG-BPEI compounds are (1) cationic for electrostatic binding to anionic sites on WTA biopolymers; (2) hydrophilic with high water solubility to reduce protein binding effects, reduce cytotoxicity from membrane permeation, and facilitate formulation into an oral, subcutaneous, or intravenous antibiotic; and (3) flexible for adapting to the disordered structure of WTA and the heterogeneous architecture of the biofilm matrix. Biofilm EPS also contains polysaccharide intercellular adhesins, such as N-acetylglucosamine (NAG), that can be cationic.

Rather than developing new inhibitors which require exhaustive clinical testing, we have identified FDA-approved β-lactam antibiotics that can regain their previously-lost efficacy against antibiotic resistant bacteria such as MRSA and other described herein. The β-lactam-BPEI combination formulations disclosed herein provide dramatic benefits to human health when used as a routine antibiotic therapy, eliminating for example S. aureus infections, while simultaneously preventing the growth of antibiotic-resistant bacteria. By using a combination of BPEI and ampicillin (or other β-lactams) to treat a non-resistant S. aureus infection, the emergence of β-lactam resistant strains in vivo can be slowed. This benefit would not be possible with ampicillin (or other β-lactams) alone.

Before further describing various embodiments of the compositions, kits and methods of the present disclosure in more detail by way of exemplary description, examples, and results, it is to be understood that the present disclosure is not limited in application to the details of methods and compositions as set forth in the following description. The description provided herein is intended for purposes of illustration only and is not intended to be construed in a limiting sense. The inventive concepts of the present disclosure are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure as defined herein. Thus, while the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference. In particular, incorporated by reference herein in their entireties are U.S. Provisional Application Ser. No. 62/747,517, filed Oct. 18, 2018, U.S. patent application Ser. No. 15/736,675, filed Dec. 14, 2017, PCT Application No. PCT/US2016/037799, filed Jun. 16, 2016, and U.S. Provisional Application Ser. No. 62/180,976, filed Jun. 17, 2015, which contain subject matter related to the present disclosure.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

Abbreviations

AMR, antimicrobial resistance; MDR, multidrug-resistant; MRSA, methicillin-resistant Staphylococcus aureus; MRSE, methicillin-resistant Staphylococcus epidermidis; BPEI, branched polyethylenimine; PEG, polyethylene glycol; LPS, lipopolysaccharide; WTA, wall teichoic acid; FICI, fractional inhibitory concentration index; EPS, extracellular polymeric substances; SEM, scanning electron microscopy; NMR, nuclear magnetic resonance; ITC, isothermal calorimetry; DMSO, dimethylsulfoxide; PBS, phosphate-buffered saline; MIC, minimum inhibitory concentration; MTD, maximum tolerable dose; PBP, penicillin-binding protein; sc, subcutaneous; MPC₄, Minimum Potentiating Concentration; MBEC, Minimum Biofilm Eradication Concentration; AuPd, Gold palladium; OXA, oxacillin; PIP, Piperacillin; PNAG, poly-N-acetyl glucosamine; PC, polycarbonate; SSTI, skin or soft-tissue infections; PmB, polymyxin B; HMDS, hexamethyldisilazane; OD₆₀₀, optical density at 600 nm; CAMHB, cation-adjusted Muller-Hinton broth; TSB, tryptic soy broth; Da, Dalton.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Use of the word “we” as a pronoun herein refers generally to laboratory personnel or other contributors who assisted in laboratory procedures and data collection and is not intended to represent an inventorship role by said laboratory personnel or other contributors in any subject matter disclosed herein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, component, step, feature, structure, or characteristic described in connection with the embodiment is included in at least one embodiment. The appearances of the phrase “in one embodiment” in various places in the specification are not necessarily all referring to the same embodiment.

The active agents of the combination therapies of the present disclosure may be used or administered conjointly. As used herein the terms “conjointly” or “conjoint administration” refers to any form of administration of two or more different biologically-active compounds (i.e., active agents) such that the second compound is administered while the previously administered therapeutic compound is still effective in the body, whereby the two or more compounds are simultaneously active in the patient, enabling a synergistic interaction of the compounds. For example, the different therapeutic compounds can be administered either in the same formulation, or in separate formulations, either concomitantly (together) or sequentially. When administered sequentially the different compounds may be administered immediately in succession, or separated by a suitable duration of time, as long as the active agents function together in a synergistic manner. In certain embodiments, the different therapeutic compounds can be administered within one hour of each other, within two hours of each other, within 3 hours of each other, within 6 hours of each other, within 12 hours of each other, within 24 hours of each other, within 36 hours of each other, within 48 hours of each other, within 72 hours of each other, or more. Thus an individual who receives such treatment can benefit from a combined effect of the different therapeutic compounds. In one example of conjoint administration, a β-lactam antibiotic and a potentiating compound (e.g., a BPEI and/or PEG-BPEI) are administered to the surface in sequential or simultaneous steps, or as a composition comprising both the β-lactam antibiotic and the potentiating compound.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability of an agent to modify the physiological system of an organism without reference to how the agent (“active agent”) has its physiological effects.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “subject” and “patient” are used interchangeably herein and will be understood to refer to a warm-blooded animal, particularly a mammal, and more particularly, humans. Animals which fall within the scope of the term “subject” as used herein include, but are not limited to, dogs, cats, rats, mice, guinea pigs, chinchillas, horses, goats, ruminants such as cattle, sheep, swine, poultry such as chickens, geese, ducks, and turkeys, zoo animals, Old and New World monkeys, and non-human primates. Veterinary diseases and conditions which may be treated with the compositions of the present disclosure include, but are not limited to, anthrax, listeriosis, leptospirosis, clostridial and corynebacterial infections, streptococcal mastitis, and keratoconjunctivitis in ruminants; erysipelas, streptococcal and clostridial infections in swine; tetanus, strangles, streptococcal and clostridial infections, and foal pneumonia in horses; urinary tract infections, and streptococcal and clostridial infections in dogs and cats; and necrotic enteritis, ulcerative enteritis and intestinal spirochetosis in poultry.

“Treatment” refers to therapeutic treatments. “Prevention” refers to prophylactic or preventative treatment measures. The term “treating” refers to administering the composition to a patient for therapeutic purposes.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “β-lactam antibiotic” refers to the class of antibiotic agents that have a β-lactam ring or derivatized β-lactam ring in their molecular structures. Examples of such β-lactam antibiotics include but are not limited to, penams, including but not limited to, penicillin, benzathine penicillin, penicillin G, penicillin V, procaine penicillin, ampicillin, amoxicillin, Augmentin® (amoxicillin+clavulanic acid), methicillin, cloxacillin, dicloxacillin, flucloxacillin, nafcillin, oxacillin, temocillin, mecillinam, carbenicillin, ticarcillin, and azlocillin, mezlocillin, piperacillin, Zosyn® (piperacillin+tazobactam); cephems, including but not limited to, cephalosporin C, cefoxitin, cephalosporin, cephamycin, cephem, cefazolin, cephalexin, cephalothin, cefaclor, cefamandole, cefuroxime, cefotetan, cefoxitin, cefixime, cefotaxime, cefpodoxime, ceftazidime, ceftriaxone, cefepime, cefpirome, and ceftaroline; carbapenems and penems including but not limited to, biapenem, doripenem, ertapenem, earopenem, imipenem, primaxin, meropenem, panipenem, razupenem, tebipenem, and thienamycin; and monobactams including but not limited to, aztreonam, tigemonam, nocardicin A, and tabtoxinine β-lactam.

The terms “effective amount”, “antibacterially-effective amount”, or “therapeutically-effective amount” refers to an amount of an antibiotic composition (β-lactam antibiotic plus BPEI, or plus PEG-BPEI) which is sufficient to exhibit a detectable therapeutic effect against bacterial growth without excessive adverse side effects (such as toxicity, irritation and allergic response) commensurate with a reasonable benefit/risk ratio when used in the manner as described herein. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance for a given subject or patient. However, the effective amount for a given situation can be determined by one of ordinary skill in the art using routine experimentation based on the information provided herein.

In some embodiments of the present disclosure a pegylated low molecular weight (“low Mw”) BPEI is used in combination with an anti-bacterial agent to treat and/or inhibit a resistant bacterial infection and/or the growth of resistant bacterial infection, e.g., by sensitizing a bacterium that was previously resistant or substantially resistant to an antibacterial agent, are described herein. In certain non-limiting embodiments the low Mw BPEI of the present disclosure has a Mw in range of, for example, 0.1 kDa (kilodaltons) to 25 kDa. Examples of BPEI compounds which may be used in various embodiments of the present disclosure include but are not limited to those shown in U.S. Pat. Nos. 7,238,451 and 9,238,716, and U.S. Published application 2014/0369953, the entireties of which are hereby incorporated by reference herein.

A minimum inhibitory concentration (MIC) of an antibiotic for a particular bacterial strain is defined as the lowest concentration of the antibiotic that is required to inhibit the growth of the bacterium. The MIC is determined by finding the concentration of antibiotic at which there is no growth of the bacterium.

A breakpoint (or resistance breakpoint) is defined as a concentration (mg/L) of an antibiotic that defines when a strain of bacteria is susceptible to successful treatment by the antibiotic. If the MIC is less than or equal to the breakpoint, the strain is considered susceptible to the antibiotic. If the MIC is greater than the breakpoint, the strain is considered intermediate or resistant to the antibiotic.

Sensitizing, or sensitization, as the term is used herein, is the process of lowering the MIC of an antibiotic for a resistant bacterial strain to a value that is below the resistance breakpoint for the bacterial strain, thereby causing the bacterium to be more susceptible to that antibiotic.

The compounds and compositions of the present disclosure can be used to treat a subject having resistant bacterial infection, e.g., by administering BPEI in combination with an antibiotic. The combinations of BPEI and the antibacterial agent can result in sensitization of a resistant bacterial strain, e.g., the resistant bacterial strain has a reduced MIC of either the BPEI, or the antibacterial agent, or both, so that the MIC is below the resistance breakpoint for the bacterial strain.

As used herein “resistant bacterial strain” means a bacterial strain which is resistant to an antibacterial agent, e.g. having an MIC that is greater than the resistance breakpoint (as the term is defined herein). In certain embodiments the MIC of a resistant bacterial strain will be at least 2-fold, 4-fold, 8-fold, 10-fold, 16-fold, 32-fold, 64-fold, or 100-fold greater than for that seen with a non-resistant bacterial strain for a selected antibacterial agent. As used herein, rendering or transforming a resistant bacterial into a sensitive bacterial strain means reducing the MIC, e.g., by at least 2-fold, 4-fold, 8-fold, 10-fold, 16-fold, 32-fold, 64-fold, or 100-fold.

The term “biofilm” as used herein refers to an aggregate of microorganisms in which cells adhere to each other and/or to a surface. These adherent cells are frequently embedded within a self-produced matrix of extracellular polymeric substance. The microorganisms comprising a biofilm may include bacteria, archaea, fungi, protozoa, algae, or combinations thereof. In particular embodiments, the biofilm comprises a bacterium (such as described elsewhere herein) such that the biofilm is a bacterial biofilm.

In some embodiments, the surface having the biofilm thereon may be a surface of a medical device. In some embodiments, the biofilm may be partially or entirely implantable in a body of a subject. For example, the medical device may be a catheter. Non-limiting examples of suitable catheters include intravascular catheters (such as, e.g., arterial catheters, central venous catheters, hemodialysis catheters, peripheral and venous catheters), endovascular catheter microcoils, peritoneal dialysis catheters, urethral catheters, catheter access ports, shunts, intubating and tracheotomy tubes. For example, the medical device may be a peripherally inserted central catheter (PICC) line. In another embodiment, the implantable device may be a cardiac device. Examples of cardiac devices include, but are not limited to, cardiac stents, defibrillators, heart valves, heart ventricular assist devices, OEM component devices, pacemakers, and pacemaker wire leads. In further embodiments, the medical device may be an orthopedic device. Non-limiting examples of suitable orthopedic devices include implants such as knee replacements, hip replacements, shoulder replacements, other joint replacements and prostheses, spinal disc replacements, orthopedic pins, plates, screws, rods, and orthopedic OEM components. In other embodiments, the medical device may include endotracheal tubes, nasogastric feeding tubes, gastric feeding tubes, synthetic bone grafts, bone cement, biosynthetic substitute skin, vascular grafts, surgical hernia mesh, embolic filter, ureter renal biliary stents, urethral slings, gastric bypass balloons, gastric pacemakers, insulin pumps, neurostimulators, penile implants, soft tissue silicone implants, intrauterine contraceptive devices, cochlear implants, dental implants and prosthetics, voice restoration devices, ophthalmic devices such as contact lenses.

In some embodiments, the surface having the biofilm thereon is a surface or within the body of a subject. For example, the subject may be a veterinary subject. Non-limiting examples of suitable veterinary subjects include companion animals such as cats, dogs, rabbits, horses, and rodents such as gerbils; agricultural animals such as cows, cattle, pigs, goats, sheep, horses, deer, chickens and other fowl; zoo animals such as primates, elephants, zebras, large cats, bears, and the like; and research animals such as rabbits, sheep, pigs, dogs, primates, chinchillas, guinea pigs, mice, rats and other rodents. For instance, the composition may be used to treat skin infections, soft tissue infections, and/or mastitis in veterinary subjects such as companion animals and/or agricultural animals. The veterinary subject may be suffering from or diagnosed with a condition needing treatment, or the veterinary subject may be treated prophylactically.

In other embodiments, the subject having the surface having the biofilm thereon may be a human health care patient. Non-limiting examples of suitable health care patients include ambulatory patients, surgery patients, medical implantation patients, hospitalized patients, long-term care patients, and nursing home patients. In still other embodiments, the subject may be a health care worker. Suitable health care workers include those with direct and indirect access to patients, medical equipment, and medical facilities.

In some embodiments, the combination of the BPEI and the antibiotic results in a reduction in the MIC of the BPEI and/or the antibiotic of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 100.5%, or more.

The antimicrobial (antibacterial) action of two or more active agents is considered additive if the combined action merely results from the addition of the effects the individual components would have in isolation. In contrast, the antimicrobial action of two or more active compounds is considered to be synergistic if the combined effect of the two or more compounds is stronger than expected based on the assumption of additivity.

The terms “synergy” or “synergistic,” as in “synergistic effect” or “synergistic activity,” refers to an effect in which two or more agents work together to produce an effect that is more than additive of the effects of each agent independently. More particularly, the terms “synergy”, “synergistic”, “synergistic effect” or “synergistic activity” as used herein, refers to an outcome when two agents (e.g., BPEI and an antibiotic) are used in combination, wherein the combination of the agents acts so as to require a smaller amount of each individual agent than would be required of that agent to be efficacious in the absence of the other agent. For example, with lower dosages of the first agent than would be required in the absence of the second agent. In some embodiments, use of synergistic agents can result in the beneficial effect of less overall use of an agent. Typically, evidence of synergistic antimicrobial action may be provided at concentrations below the MICs of each of the components when taken individually. However, a synergistic interaction can also occur when the concentration of one or more of the active compounds is raised above its MIC (when taken individually).

The fractional inhibitory concentration (FIC) as used herein is a measure of the interaction of two agents, such as an antibiotic and a BPEI compound, used together, and is an indicator of synergy. FIC uses a value of the MIC of each of the independent agents, e.g., MIC_{A} and MIC_{B} for agents A and B, for a particular bacterium as the basis, then takes the concentration of each component in a mixture where an MIC_{(A in B)} is observed. For example, for a two component system of agents A and B, MIC_{(A in B)} is the concentration of A in the compound mixture and MIC_{(B in A)} is the concentration of B in the compound mixture. The FIC is defined as follows:

FIC_{A}=(MIC_{(A in B)}/MIC_{A}) Eqn. 1

FIC_{B}=(MIC_{(B in A)}/MIC_{B}) Eqn. 2

FIC_{A+3}=FIC_{A}+FIC_{B} Eqn. 3

Synergism (i.e., the two compounds together provide a synergistic effect or synergistic activity against a bacterium) is defined herein as occurring when FIC_{A+B}≤0.5. The mixture is defined as having an additive effect when 1≤FIC_{A+B}≤4. When, FIC_{A+B}>4 the mixture is considered to have an antagonistic interaction. An example of how FIC is used to determine synergism is shown in U.S. Pat. No. 8,338,476, the entirety of which is incorporated herein by reference in its entirety.

In certain embodiments of the present disclosure, the BPEI/antibiotic or PEG-BPEI/antibiotic combination results in an FIC less than about 0.55, or less than about 0.5, or less than about 0.4, or less than about 0.3, or less than about 0.2, or less than about 0.1, or less than about 0.05, or less than about 0.02, or less than about 0.01, or less than about 0.005, or less than about 0.001. In some embodiments, the combination results in a bactericidal activity at least about 2 logs, at least about 2.5 logs, at least about 3 logs, at least about 3.5 logs, at least about 4 logs, at least about 4.5 logs, or at least about 5 logs more effective than the most effective individual activity, e.g., the activity of the BPET or the antibiotic agent.

As used herein, “resistant microorganism or bacterium” means an organism which has become resistant to an anti-bacterial agent. In certain embodiments an MIC of a resistant bacterium will be at least, 2-fold, 4-fold, 8-fold, 10-fold, 16-fold, 32-fold, 64-fold, or 100-fold greater than that seen with a non-resistant bacterium for a particular anti-bacterial agent. As used herein, the term “resistance breakpoint” is the threshold concentration of an antibacterial agent above which a bacterium is considered resistant, as defined above.

In certain non-limiting embodiments, the antibiotic/BPEI composition is formulated to contain a mass ratio in a range of 100:1 (e.g., 100 mg antibiotic per 1 mg of BPEI additive), to 1:1 (1 mg antibiotic per 1 mg BPEI), to 1:100 (1 mg antibiotic per 100 mg BPEI), or more particularly, a mass ratio in a range of 75:1 (e.g., 75 mg antibiotic per 1 mg of BPEI additive), to 1:1 (1 mg antibiotic per 1 mg BPEI), to 1:75 (1 mg antibiotic per 75 mg BPEI), or more particularly, a mass ratio in a range of 64:1 (e.g., 64 mg antibiotic per 1 mg of BPEI additive), to 1:1 (1 mg antibiotic per 1 mg BPEI), to 1:64 (1 mg antibiotic per 64 mg BPEI), or more particularly, a mass ratio in a range of 50:1 (e.g., 50 mg antibiotic per 1 mg of BPEI additive), to 1:1 (1 mg antibiotic per 1 mg BPEI), to 1:50 (1 mg antibiotic per 50 mg BPEI), or more particularly, a mass ratio in a range of 32:1 (e.g., 32 mg antibiotic per 1 mg of BPEI additive), to 1:1 (1 mg antibiotic per 1 mg BPEI), to 1:32 (1 mg antibiotic per 32 mg BPEI), or more particularly, a mass ratio in a range of 24:1 (e.g., 24 mg antibiotic per 1 mg of BPEI additive), to 1:1 (1 mg antibiotic per 1 mg BPEI), to 1:24 (1 mg antibiotic per 24 mg BPEI), or more particularly, a mass ratio in a range of 16:1 (e.g., 16 mg antibiotic per 1 mg of BPEI additive), to 1:1 (1 mg antibiotic per 1 mg BPEI), to 1:16 (1 mg antibiotic per 16 mg BPEI), or more particularly, a mass ratio in a range of 10:1 (e.g., 10 mg antibiotic per 1 mg of BPEI additive), to 1:1 (1 mg antibiotic per 1 mg BPEI), to 1:10 (1 mg antibiotic per 10 mg BPEI), or more particularly, a mass ratio in a range of 8:1 (e.g., 8 mg antibiotic per 1 mg of BPEI additive), to 1:1 (1 mg antibiotic per 1 mg BPEI), to 1:8 (1 mg antibiotic per 8 mg BPEI), or more particularly, a mass ratio in a range of 4:1 (e.g., 4 mg antibiotic per 1 mg of BPEI additive), to 1:1 (1 mg antibiotic per 1 mg BPEI), to 1:4 (1 mg antibiotic per 4 mg BPEI), or any range comprising a combination of said ratio endpoints, such as for example, a mass ratio in a range of 64:1 (e.g., 64 mg antibiotic per 1 mg of BPEI additive), to 1:4 (1 mg antibiotic per 4 mg BPEI), or a mass ratio in a range of 32:1 (e.g., 32 mg antibiotic per 1 mg of BPEI additive), to 1:16 (1 mg antibiotic per 16 mg BPEI).

In certain non-limiting embodiments, the dosage of the antibiotic/BPEI composition administered to a subject could be in a range of 1 μg per kg of subject body mass to 1000 mg/kg, or in a range of 5 μg per kg to 500 mg/kg, or in a range of 10 μg per kg to 300 mg/kg, or in a range of 25 μg per kg to 250 mg/kg, or in a range of 50 μg per kg to 250 mg/kg, or in a range of 75 μg per kg to 250 mg/kg, or in a range of 100 μg per kg to 250 mg/kg, or in a range of 200 μg per kg to 250 mg/kg, or in a range of 300 μg per kg to 250 mg/kg, or in a range of 400 μg per kg to 250 mg/kg, or in a range of 500 μg per kg to 250 mg/kg, or in a range of 600 μg per kg to 250 mg/kg, or in a range of 700 μg per kg to 250 mg/kg, or in a range of 800 μg per kg to 250 mg/kg, or in a range of 900 μg per kg to 250 mg/kg, or in a range of 1 mg per kg to 200 mg/kg, or in a range of 1 mg per kg to 150 mg/kg, or in a range of 2 mg per kg to 100 mg/kg, or in a range of 5 mg per kg to 100 mg/kg, or in a range of 10 mg compound per kg to 100 mg/kg, or in a range of 25 mg per kg to 75 mg/kg. For example, in certain non-limiting embodiments, the composition could contain antibiotic in a range of 0.1 mg/kg to 10 mg/kg, and BPEI in a range of 0.1 mg/kg to 10 mg/kg, or any range comprising a combination of said ratio endpoints, such as, for example, a range of 10 μg/kg to 10 mg/kg of the antibiotic/BPEI composition. In some embodiments, the antibiotic and/or potentiating compound is administered at a dose of about 0.1 mg/kg to about 50 mg/kg. In particular embodiments, the subject is a pediatric patient, which means under 18 years of age for a human patient. For a pediatric patient, in some embodiments the antibiotic and/or potentiating compound is administered about 10 mg/kg to about 50 mg/kg intravenously or intramuscularly every 6 to 12 hours or about 12.5 mg/kg orally every 6 hours.

The BPEI used in the present formulations may have an average molecular weight (MW) in a range of, for example, from 0.1 kDa (kilodaltons), to 0.2 kDa, to 0.3 kDa, to 0.4 kDa, to 0.50 kDa, to 0.6 kDa, to 0.7 kDa, to 0.8 kDa, to 0.9 kDa, to 1.0 kDa, to 1.1 kDa, to 1.2 kDa, to 1.3 kDa, to 1.4 kDa, to 1.5 kDa, to 1.6 kDa, to 1.7 kDa, to 1.8 kDa, to 1.9 kDa, to 2 kDa, to 2.5 kDa, to 3 kDa, to 3.5 kDa, to 4 kDa, to 4.5 kDa, to 5 kDa, to 5.5 kDa, to 6 kDa, to 6.5 kDa, to 7 kDa, to 7.5 kDa, to 8 kDa, to 9 kDa, to 10 kDa, to 12.5 kDa, to 15 kDa, to 17.5 kDa, to 20 kDa, to 22.5 kDa, to 25 kDa, to 30 kDa, to 35 kDa, to 40 kDa, to 45 kDa, to 50 kDa, to 55 kDa, to 60 kDa, to 65 kDa, to 70 kDa, to 75 kDa including any fractional or integeric value within said range. Also, the percentage of primary amine-to-secondary amine-to-tertiary amine in the BPEI can be varied. For example, the BPEI may have a higher primary amine content as compared to the secondary amine and/or tertiary amine content.

The PEG molecules used in the present formulations may have an average molecular weight (MW) in a range of, for example, from 0.1 kDa (kilodaltons), to 0.2 kDa, to 0.3 kDa, to 0.4 kDa, to 0.50 kDa, to 0.6 kDa, to 0.7 kDa, to 0.8 kDa, to 0.9 kDa, to 1.0 kDa, to 1.1 kDa, to 1.2 kDa, to 1.3 kDa, to 1.4 kDa, to 1.5 kDa, to 1.6 kDa, to 1.7 kDa, to 1.8 kDa, to 1.9 kDa, to 2 kDa, to 2.1 kDa, to 2.2 kDa, to 2.3 kDa, to 2.4 kDa, to 2.5 kDa, to 2.6 kDa, to 2.7 kDa, to 2.8 kDa, to 2.9 kDa, to 3 kDa, to 3.1 kDa, 3.2 kDa, to 3.3 kDa, to 3.4 kDa, to 3.5 kDa, to 3.6 kDa, to 3.7 kDa, to 3.8 kDa, to 3.9 kDa, to 4 kDa, to 4.1 kDa, 4.2 kDa, to 4.3 kDa, to 4.4 kDa, to 4.50 kDa, to 4.6 kDa, to 4.7 kDa, to 4.8 kDa, to 4.9 kDa, to 5 kDa, to 5.5 kDa, to 6 kDa, to 6.5 kDa, to 7 kDa, to 7.5 kDa, to 8 kDa, to 9 kDa, to 10 kDa, including any fractional or integeric value within said range, such as 150 Da to 2500 Da (i.e., 0.15 kDa to 2.5 kDa), 200 Da to 1750 Da (i.e., 0.2 kDa to 1.75 kDa), 250 Da to 1500 Da (i.e., 0.25 kDa to 1.5 kDa), and 300 Da to 1250 Da (i.e., 0.3 kDa to 1.25 kDa).

The antibiotic and BPEI or PEG-BPEI can be administered conjointly, i.e., together in a single formulation (dose), or together (simultaneously) in separate formulations (doses), or sequentially, whereby administration of the antibiotic dosage is followed by the BPEI dosage, or administration of the BPEI dosage is followed by administration of the antibiotic dosage. The dosage(s) can be administered, for example but not by way of limitation, on a one-time basis, or administered at multiple times (for example but not by way of limitation, from one to five times per day, or once or twice per week), or continuously via a venous drip, depending on the desired therapeutic effect. In one non-limiting example of a therapeutic method of the present disclosure, the composition is provided in an IV infusion. Administration of the compounds used in the pharmaceutical composition or to practice the method of the present disclosure can be carried out in a variety of conventional ways, such as, but not limited to, orally, by inhalation, rectally, or by cutaneous, subcutaneous, intraperitoneal, vaginal, or intravenous injection. Oral formulations may be formulated such that the compounds pass through a portion of the digestive system before being released, for example it may not be released until reaching the small intestine, or the colon.

In some embodiments the antibiotic and the potentiator compound are in the same composition. In other embodiments the antibiotic and the potentiator compound are administered simultaneously in the same or different compositions. A subject is administered an antibiotic up to 24 hours prior to administration of the potentiator compound in some cases. In others, the potentiator compound is administered up to 24 hours prior to administration of the antibiotic. In some embodiments, the antibiotic and potentiator compound are administered within 0.5, 1, 2, 3, 4, 6, 8, 10, 12, 14, 16, 18, 20, 22, or 24 hours of each other.

As noted above, in certain embodiments, the compositions of the present disclosures may be applied topically to an external or internal wound to treat a planktonic or biofilm bacterial infection in or on the wound. The treated wounds may be acute wounds, such as abrasions, lacerations, punctures, avulsions and incisions, or chronic wounds, or “non-healing” wounds such as diabetic foot ulcers, venous leg ulcers, pressure ulcers (e.g., bed sores), wounds due to arterial insufficiency, radiation wounds, and non-healing surgical wounds (e.g., due to abdominal surgery).

The composition for topical or internal application may be provided in any suitable solid, semi-solid, or liquid form. In certain embodiments, the topical composition may be provided in or be disposed in a carrier(s) or vehicle(s) such as, for example, creams, pastes, gums, lotions, gels, foams, ointments, emulsions, suspensions, aqueous solutions, powders, lyophilized powders, solutions, granules, foams, drops, eye drops, adhesives, sutures, aerosols, sprays, sticks, soaps, bars of soap, balms, body washes, rinses, tinctures, gel beads, gauzes, wound dressings, bandages, cloths, towelettes, stents, and sponges. Non-limiting examples of formulations of such carriers and vehicles include, but are not limited to, those shown in “Remington, The Science and Practice of Pharmacy, 22nd ed., 2012, edited by Loyd V. Allen, Jr”.

Creams are emulsions of water in oil (w/o), or oil in water (o/w). O/w creams spread easily and do not leave the skin greasy and sticky. W/o creams tend to be more greasy and more emollient. Ointments are semi-solid preparations of hydrocarbons and the strong emollient effect makes it useful in cases of dry skin. The occlusive effect enhances penetration of the active agent and improves efficacy. Pastes are mixtures of powder and ointment. The addition of the powder improves porosity thus breathability. The addition of the powder to the ointment also increases consistency so the preparation is more difficult to rub off or contact non-affected areas of the skin. Lotions are liquid preparations in which inert or active medications are suspended or dissolved. For example, an o/w emulsion with a high water content gives the preparation a liquid consistency of a lotion. Most lotions are aqueous of hydroalcoholic systems wherein small amounts of alcohol are added to aid in solubilization of the active agent and to hasten evaporation of the solvent from the skin surface. Gels are transparent preparations containing cellulose ethers or carbomer in water, or a water-alcohol mixture. Gels liquefy on contact with the skin, dry, and leave a thin film of active medication.

A person with ordinary skill in the art will be capable of determining the effective amount of the composition needed for a particular treatment. Such amount may depend on the strength of the composition or extent of the wound to be treated. Although a person with ordinary skill in the art will know how to select a treatment regimen for a specific condition. In a non-limiting example, a dosage of the composition comprising about 0.01 mg to about 1000 mg of the active agent (antibiotic plus BPEI or PEG+BPEI) per ml may be applied 1 to 2 to 3 to 4 to 5 to 6 times per day or more to the affected area. It is foreseeable in some embodiments that the composition is administered over a period of time. The composition may be applied for a day, multiple days, a week, multiple weeks, a month, or even multiple months in certain circumstances. Alternatively, the composition may be applied only once when the skin condition is mild.

In certain embodiments, the composition may comprise the active agents in a concentration of, but is not limited to, 0.0001 M to 1 M, for example, or 0.001 M to 0.1 M. The composition may comprise about 0.01 to about 1000 milligrams of the active agents per ml of carrier or vehicle with which the active agents are combined in a composition or mixture. The composition may comprise about 1 wt % to about 90 wt % (or 1 mass % to about 90 mass %) of one or more shikimate analogues and about 10 wt % to about 99 wt % (or 10 mass % to about 99 mass %) of one or more secondary compounds (where “wt %” is defined as the percentage by weight of a particular compound in a solid or liquid composition, and “mass %” is defined as the percentage by mass of a particular compound in a solid or liquid composition).

The topical compositions may further comprise ingredients such as propylene glycol, sodium stearate, glycerin, a surfactant (e.g., sodium laurate, sodium laureth sulfate, and/or sodium lauryl sulfate), and water, and optionally, sorbitol, sodium chloride, stearic acid, lauric acid, aloe vera leaf extract, pentasodium penetrate, and/or tetrasodium etidronate.

The topical compositions may be formulated with liquid or solid emollients, solvents, thickeners, or humectants. Emollients include, but are not limited to, stearyl alcohol, mink oil, cetyl alcohol, oleyl alcohol, isopropyl laurate, polyethylene glycol, olive oil, petroleum jelly, palmitic acid, oleic acid, and myristyl myristate. Emollients may also include natural butters extracted from various plants, trees, roots, or seeds. Examples of such butters include, but are not limited to, shea butter, cocoa butter, avocado butter, aloe butter, coffee butter, mango butter, or combination thereof.

Suitable materials which may be used in the compositions as carriers or vehicles or secondary compounds or solvents include, but are not limited to, propylene glycol, ethyl alcohol, isopropanol, acetone, diethylene glycol, ethylene glycol, dimethyl sulfoxide, and dimethyl formamide. Suitable humectants include, but are not limited to, acetyl arginine, algae extract, Aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxypolyethylene glycol, myristamidobutyl guanidine acetate, polyglyceryl sorbitol, potassium pyrollidone carboxylic acid (PCA), propylene glycol (PGA), sodium pyrollidone carboxylic acid (PCA), sorbitol, and sucrose. Other humectants may be used for yet additional embodiments of the compositions of the present disclosure.

Suitable thickeners include, but are not limited to, polysaccharides, in particular xantham gum, guar-guar, agar-agar, alginates, carboxymethylcellulose, relatively high molecular weight polyethylene glycol mono- and diesters of fatty acids, polyacrylates, polyvinyl alcohol and polyvinylpyrrolidone, surfactants such as, for example, ethoxylated fatty acid glycerides, esters of fatty acids with polyols such as, for example, pentaerythritol or trimethylpropane, fatty alcohol ethoxylates or alkyl oligoglucosides, and electrolytes, such as sodium chloride and ammonium chloride.

The topical compositions may further comprise one or more penetrants, compounds facilitating penetration of active ingredients into the skin of a patient. Non-limiting examples of suitable penetrants include isopropanol, polyoxyethylene ethers, terpenes, cis-fatty acids (oleic acid, palmitoleic acid), acetone, laurocapram dimethyl sulfoxide, 2-pyrrolidone, oleyl alcohol, glyceryl-3-stearate, cholesterol, myristic acid isopropyl ester, and propylene glycol. Additionally, the compositions may include surfactants or emulsifiers for forming emulsions. Either a water-in-oil or oil-in-water emulsion may be formulated. Examples of suitable emulsifiers include, but are not limited to, stearic acid, cetyl alcohol, PEG-100, stearate and glyceryl stearate, cetearyl glucoside, polysorbate 20, methylcellulose, sodium carboxymethylcellulose, glycerin, bentonite, ceteareth-20, cetyl alcohol, cetearyl alcohol, lanolin alcohol, riconyl alcohol, self-emulsifying wax (e.g., Lipowax P), cetyl palmitate, stearyl alcohol, lecithin, hydrogenated lecithin, steareth-2, steareth-20, and polyglyceryl-2 stearate.

In some formulations, such as in aerosol form, the composition may also include a propellant. For example, hydrofluoroalkanes (HFA) such as either HFA 134a (1,1,1,2-tetrafluoroethane) or HFA 227 (1,1,1,2,3,3,3-heptafluoropropane) or combinations of the two, may be used since they are widely used in medical applications. Other suitable propellants include, but are not limited to, mixtures of volatile hydrocarbons, typically propane, n-butane and isobutane, dimethyl ether (DME), methylethyl ether, nitrous oxide, and carbon dioxide. Those skilled in the art will readily appreciate that emollients, solvents, thickeners, humectants, penetrants, surfactants or emulsifiers, and propellants, other than those listed may also be employed.

When a therapeutically effective amount of the composition(s) is administered orally, it may be in the form of a solid or liquid preparation such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition(s) may contain a solid carrier, such as a gelatin or an adjuvant. The tablet, capsule, and powder may contain from about 0.05 to about 95% of the active substance compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition(s) may further contain physiological saline solution, dextrose or other saccharide solution, or glycols such as ethylene glycol, propylene glycol, or polyethylene glycol. When administered in liquid form, the pharmaceutical composition(s) particularly contains from about 0.005 to about 95% by weight of the active substance. For example, a dose of about 10 mg to about 1000 mg once or twice a day could be administered orally.

In another embodiment, the composition(s) of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the composition(s) in an aqueous or non-aqueous pharmaceutically acceptable solvent which may also contain suspending agents, sweetening agents, flavoring agents, and preservative agents as are known in the art.

For parenteral administration, for example, the composition(s) may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When a therapeutically effective amount of the composition(s) is administered by intravenous, cutaneous, or subcutaneous injection, the compound is particularly in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the active agent(s), an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition(s) of the present disclosure may also contain stabilizers, preservatives, buffers, antioxidants, or other additives known to those of skill in the art.

As noted, particular amounts and modes of administration can be determined by one skilled in the art. One skilled in the art of preparing formulations can readily select the proper form and mode of administration, depending upon the particular characteristics of the composition(s) selected, the infection to be treated, the stage of the infection, and other relevant circumstances using formulation technology known in the art, described, for example, in Remington: The Science and Practice of Pharmacy, 22^{nd} ed.

Additional pharmaceutical methods may be employed to control the duration of action of the composition(s). Increased half-life and/or controlled release preparations may be achieved through the use of polymers to conjugate, complex with, and/or absorb the active substances described herein. The controlled delivery and/or increased half-life may be achieved by selecting appropriate macromolecules (for example but not by way of limitation, polysaccharides, polyesters, polyamino acids, homopolymers polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release. The compound(s) may also be ionically or covalently conjugated to the macromolecules described above.

Another possible method useful in controlling the duration of action of the composition(s) by controlled release preparations and half-life is incorporation of the composition(s) or functional derivatives thereof into particles of a polymeric material such as polyesters, polyamides, polyamino acids, hydrogels, poly(lactic acid), ethylene vinylacetate copolymers, copolymer micelles of, for example, PEG and poly(l-aspartamide).

Examples of bacterial families which contain bacterial species against which the presently disclosed compositions and treatment protocols are effective include, but are not limited to: Alicyclobacillaceae, Bacillaceae, Listeriaceae, Paenibacillaceae, Pasteuriaceae, Planococcaceae, Sporolactobacillaceae, Staphylococcaceae, Thermoactinomycetaceae, Aerococcaceae, Carnobacteriaceae, Enterococcaceae, Lactobacillaceae, Leuconostocaceae, Streptococcaceae, Caldicoprobacteraceae, Christensenellaceae, Clostridiaceae, Defluviitaleaceae, Eubacteriaceae, Graciibacteraceae, Heliobacteriaceae, Lachnospiraceae, Oscillospiraceae, Peptococcaceae, Peptostreptococcaceae, Ruminococcaceae, Syntrophomonadaceae, Veillonellaceae, Halanaerobiaceae, Halobacteroidaceae, Natranaerobiaceae, Thermoanaerobacteraceae, and Thermodesulfobiaceae.

Specific bacteria that can be treated with the compositions and methods of the present disclosure include, but are not limited to: Enterococcus faecalis, Enterococcus faecium, Staphylococcus aureus, Methicillin-resistant Staphylococcus aureus (MRSA), Staphylococcus epidermidis, Methicillin-resistant Staphylococcus epidermidis (MRSE), oxacillin-resistant Staphylococcus aureus (ORSA), vancomycin-resistant Staphylococcus aureus (VRSA), Streptococcus pneumonia, e.g., penicillin-resistant Streptococcus pneumonia, Streptococcus mutans, Streptococcus sanguinis, Bacillus anthracis, Bacillus cereus, Clostridium botulinum, Clostridium botulinum, Pseudomonas aeruginosa, MDR Pseudomonas aeruginosa, and Listeria monocytogenes.

In certain embodiments, the compositions of the present disclosure may be provided as a package or kit, which include, for example, substantially pure preparations of the active agents described herein, combined with pharmaceutically acceptable carriers, diluents, solvents, excipients, and/or vehicles to produce an appropriate pharmaceutical composition. One embodiment of such a package or kit therefore includes at least one container with an antibiotic and at least one container with a potentiating compound. Each container may comprise a pharmaceutically acceptable carrier, diluent, solvent, excipient, and/or vehicle. Each container may comprise one or more doses of the antibiotic and/or of the potentiating compound. The package or kit may comprise a plurality of containers with an antibiotic and a potentiating compound. The package or kit may comprise a plurality of containers each with a different an antibiotic and a plurality of containers with the same potentiating compound or different potentiating compounds. The package or kit may further comprise a set of directions for administering the antibiotic(s) and potentiating compound(s).

In some embodiments, the present disclosure is directed to apparatus such as medical devices and medical instruments which have been externally and/or internally coated with a potentiated antibiotic composition as described herein. For example, the potentiated antibiotic composition may be combined with a biodegradable or dissolvable polymeric material to form the material used to make the coating. For example, the medical device may be a catheter. Non-limiting examples of suitable catheters include intravascular catheters (such as, e.g., arterial catheters, central venous catheters, hemodialysis catheters, peripheral and venous catheters), endovascular catheter microcoils, peritoneal dialysis catheters, urethral catheters, urinary catheters, catheter access ports, shunts, intubating and tracheotomy tubes. For example, the medical device may be a PICC line. In another embodiment, the device may be an implabtable cardiac device. Examples of cardiac devices include, but are not limited to, cardiac stents, defibrillators, heart valves, heart ventricular assist devices, OEM component devices, pacemakers, and pacemaker wire leads. In further embodiments, the medical device may be an orthopedic device. Non-limiting examples of suitable orthopedic devices include implants such as knee replacements, hip replacements, shoulder replacements, other joint replacements and prostheses, spinal disc replacements, orthopedic pins, plates, screws, rods, and orthopedic OEM components. In other embodiments, the medical device may include endotracheal tubes, nasogastric feeding tubes, gastric feeding tubes, synthetic bone grafts, bone cement, biosynthetic substitute skin, vascular grafts, surgical hernia mesh, embolic filter, ureter renal biliary stents, urethral slings, gastric bypass balloons, gastric pacemakers, insulin pumps, neurostimulators, penile implants, soft tissue silicone implants, intrauterine contraceptive devices, cochlear implants, dental implants and prosthetics, voice restoration devices, and ophthalmic devices such as contact lenses.

### EXAMPLES

The inventive concepts of the present disclosure will now be discussed in terms of several specific, non-limiting, examples. The examples described below, which include particular embodiments, will serve to illustrate the practice of the present disclosure, it being understood that the particulars shown are by way of example and for purposes of illustrative discussion of particular embodiments of the present disclosure only and are presented in the cause of providing what is believed to be a useful and readily understood description of procedures as well as of the principles and conceptual aspects of the inventive concepts.

### Example 1: Antibiotic Synergies of β-Lactam/BPEI and β-Lactam/PEG-BPEI

In non-limiting embodiment, a combined antibiotic drug+BPEI or combined antibiotic drug+PEG BPEI therapy can be used to enhance the efficacy of any antimicrobial used against bacteria that are growing, non-growing, stationary, or dormant within biofilms. In particular, the PEG-BPEIs of the disclosure potentiate antibiotics against MRSA and MRSE. The PEG-BPEIs of the disclosure bind to WTA and thus, prevent PBP2a and PBP4 from functioning properly. Additionally, the in vitro effective concentration of the PEG-BPEIs is orders of magnitude lower than the in vitro cytotoxic concentration. Binding of the PEG to the BPEI as a co-polymer increases the maximum tolerable dose. For example, the in vivo maximum tolerable dose (MTD or LD₀) of PEG₁₀₀₀BPEI₆₀₀ in mice with subcutaneous dosing is greater than 200 mg/kg which is at least 8 times higher than that of 600-Da BPEI (25 mg/kg).

The compositions and methods of the present disclosure therefore are intended to be used to potentiate antibiotics such as β-lactams, vancomycin, linezolid, rifampicin against their target bacterial pathogens such as MRSA and MRSE that express biofilm extracellular polymeric substances (EPS) and the mecA gene responsible for PBP2a expression. Without wishing to be bound by theory, it is believed that resistance from EPS and PBP2a can be conquered when WTA is disabled by cationic polymer potentiators such as BPEI. This effect may arise from electrostatic interactions between BPEI and WTA which disrupt the biofilm architecture and counteract resistance from mecA, making MRSA and MRSE susceptible to the antibiotics. Toxicity of the BPEI is reduced by linkage to PEG.

As noted, MRSE and MRSA rely on PBP2a to survive in the presence of β-lactam antibiotics and have become a serious threat to public health. Diagnosed or suspected MRSA infections require treatment with vancomycin, linezolid, or daptomycin, and to date, no MRSA strain is currently resistant to more than one of them. Other drugs such as ceftaroline, teflaro, and telavancin have been approved for patient use in severe cases but all must be given intravenously. Yet, when these patients are admitted to hospital, they are given vancomycin. Vancomycin is not without risk and linezolid is restricted to short or intermediate usage as it causes mitochondrial toxicity, especially dangerous for dialysis patients. Results below shows the effect of cationic polymer potentiators on resistance from PBP2a and biofilm EPS.

In the present work, low MW BPEI polymers are used as antibiotic adjuvants. Using the disclosed compositions, WTA biosynthesis still occurs naturally in bacteria but is deactivated in situ through electrostatic interactions with the BPEI. This enables the simultaneous disabling of the WTA in the biofilm EPS as well as within the cell wall.

Without wishing to be bound by theory it is proposed that cationic polymer potentiators interact with anionic WTA using amine-phosphate binding. We used NMR spectroscopy to study the structure of WTA and changes caused by metal ions. We have also examined the equilibrium binding behavior of Ca²⁺ and Mg²⁺ with WTA and described a metal-to-WTA binding mechanism. As shown in U.S. patent application Ser. No. 15/736,675, PCT Application No. PCT/US2016/037799, and U.S. Provisional Application Ser. No. 62/180,976, small amounts (e.g., 1-8 μg/mL) of BPEI potentiate β-lactam antibiotics against MRSA. The effect of BPEI and β-lactam antibiotics in inhibiting MRSA growth is characterized as synergistic from the FIC index and determination of MBC values. Monitoring MRSA growth reveals that bacteria exposed to sub-inhibitory concentrations of BPEI and oxacillin fail to reach exponential phase when the two compounds are combined. This data demonstrates that the mechanism by which BPEI+oxacillin prevents growth of MRSA is bactericidal. Additional checkerboard assays shown herein demonstrate anti-MRSA potency of β-lactam antibiotics mixed with BPEI₆₀₀ when exposed to MRSA USA300, the predominant epidemic MRSA strain (Table 1). Higher BPEI₆₀₀ concentrations decrease further the MIC values.

Our data support a WTA-based mechanism by the absence of potentiation in a MRSA-MW2 ΔtarO strain which lacks WTA, the presumed target for BPEI binding. As recently reported, BPEI₆₀₀ does not alter the oxacillin MIC value against the mutant (the expected result if WTA is the BPEI target) and SEM images of BPEI-treated MRSA, collected at mid-exponential phase, are similar to those of the WTA-deficient mutant (Foxley, M. A.; Wright, S. N.; Lam, A. K.; Friedline, A. W.; Strange, S. J.; Xiao, M. T.; Moen, E. L.; Rice, C. V., Targeting Wall Teichoic Acid in Situ with Branched Polyethylenimine Potentiates beta-Lactam Efficacy against MRSA. ACS Med. Chem. Let. 2017, 8 (10), 1083-1088). We previously reported ³¹P NMR spectra whose perturbations are explained with phosphate-amine-binding and fluorescent laser-scanning confocal-microscopy (LSCM) images showing that cationic polymer potentiators binding to the cell wall and septum regions where WTA is located (Foxley, M. A.; Friedline, A. W.; Jensen, J. M.; Nimmo, S. L.; Scull, E. M.; King, J. B.; Strange, S.; Xiao, M. T.; Smith, B. E.; Thomas Iii, K. J.; Glatzhofer, D. T.; Cichewicz, R. H.; Rice, C. V., Efficacy of ampicillin against methicillin-resistant Staphylococcus aureus restored through synergy with branched poly(ethylenimine). J Antibiot (Tokyo) 2016, 69 (12), 871-878).

Without wishing to be bound by theory, it is believed that the effect cationic polymers have on the potentiation of the effect of antibiotics against exopolymers has a technical foundation from BPEI potentiation of antibiotics against MRSE. S. epidermidis ATCC 12228®, a negative control which does not contain the mecA or ica gene, is susceptible to β-lactam antibiotics and does not have a slime exolayer. As summarized in Table 2A, BPEI₆₀₀ does not alter the MICs of oxacillin, vancomycin, or linezolid. However, the presence of mecA and ica in the MRSE strain S. epidermidis ATCC 35984® confers resistance from PBP2a and extracellular slime, respectively. These effects are shown in Table 2B where MRSE 35984 shows higher MICs for oxacillin, vancomycin, and linezolid. By adding 6.75 μM of BPEI₆₀₀, the MIC of oxacillin is reduced 256-fold, an effect that is superior to the 2-fold reduction seen with MRSA USA300 (Table 1). Unlike the data for MRSA USA300, we also observed potentiation of vancomycin and linezolid against MRSE 35984. Thus, in addition to overcoming resistance from mecA, BPEI₆₀₀ can reduce resistance from the slime layer. The potentiation of oxacillin, vancomycin, and linezolid by BPEI₆₀₀ is characterized as synergistic from the fractional inhibitory concentration (FIC) index and determination of MBC values. Biofilms were created in the bottom of 96-well plates as confirmed by crystal violet staining. However, measuring MBEC requires several rinsing and media replacement steps that cause mechanical disruption of the biofilm. The Calgary Biofilm Device, with plastic pegs on the plate lid, is designed for robust and reproducible measurement of MBEC (Ceri, H.; Olson, M. E.; Stremick, C.; Read, R. R.; Morck, D.; Buret, A., The Calgary Biofilm Device: New technology for rapid determination of antibiotic susceptibilities of bacterial biofilms. Journal of Clinical Microbiology 1999, 37 (6), 1771-1776; and Harrison, J. J.; Ceri, H.; Yerly, J.; Stremick, C. A.; Hu, Y. P.; Martinuzzi, R.; Turner, R. J., The use of microscopy and three-dimensional visualization to evaluate the structure of microbial biofilms cultivated in the Calgary Biofilm Device. Biological Procedures Online 2006, 8, 194-215)

BPEI has been PEGylated for gene delivery (Kim, S. J.; Singh, M.; Wohlrab, A.; Yu, T. Y.; Patti, G. J.; O'Connor, R. D.; VanNieuwenhze, M.; Schaefer, J., The isotridecanyl side chain of plusbacin-A3 is essential for the transglycosylase inhibition of peptidoglycan biosynthesis. Biochemistry 2013, 52 (11), 1973-9), and toxicity data shows that PEGylation of cationic amine polymers reduces toxicity. Non-toxic PEG is FDA approved for pharmaceuticals and PEG functionalized drugs are used clinically for a variety of diseases. The approach disclosed herein employs PEGylated-BPEI as a potentiator rather than as an antibiotic itself.

Checkerboard assays demonstrate anti-MRSA potency of β-lactam antibiotics mixed with BPEI₆₀₀ when exposed to MRSA USA300, the predominant epidemic MRSA strain (Table 1). The USCAST definition of oxacillin resistance is an MIC>2 μg/mL and the cut-offs for other penicillins are referenced to this value. Resistance is also removed for cephalosporins and imipenem. We have observed potentiation in fetal bovine serum (FBS) which suggests minimal protein binding effects, 50% FBS does not change the oxacillin MIC measured in CAMHB without FBS. This indicates that serum proteins do not hinder potentiation from BPEI.

As shown in the checkerboard assay data of Tables 1 and 3, PEG-BPEI copolymers potentiate the activity of β-lactam antibiotics. These data show that modification of BPEI with a single PEG₃₅₀ molecule does not prevent potentiation against MRSA. Data for MRSA USA300 show an oxacillin MIC of 1 μg/mL with 4 μg/mL BPEI₆₀₀ (6.75 μM, Table 1) and an oxacillin MIC of 0.5 μg/mL with 16 μg/mL of PEG₃₅₀BPEI₆₀₀ (17 μM, Table 3A). The amounts of potentiator are similar whereas modification of BPEI with PEG₁₀₀₀ reduces potentiation by 3× in μM units. An oxacillin MIC of 0.5 μg/ml requires 64 μg/mL of PEG₁₀₀₀BPEI₆₀₀ (40 μM, Table 3B). Thus, PEGylation with 1000 MW PEG (PEG₁₀₀₀) lowers potentiation. Although PEG₁₀₀₀BPEI₆₀₀ has lower efficacy than PEG₃₅₀BPEI₆₀₀ or BPEI₆₀₀ itself, PEGylation increases safety by lowering toxicity.

Capping one primary amine on BPEI₆₀₀ with a single PEG₃₅₀ molecule retains the ability to remove β-lactam resistance in MRSA. Data for MRSA USA300 show an oxacillin MIC of 1 μg/mL with 8 μg/mL (8.5 μM, Table 4) of PEG₃₅₀-BPEI₆₀₀ whereas modification of BPEI with PEG₁₀₀₀ reduces potentiation by 3× in μM units. An oxacillin MIC of 1 μg/mL requires 32 μg/mL (20 μM) of PEG₁₀₀₀-BPEI₆₀₀.

The BPEI compounds of the present disclosure disable resistance in Gram-negative bacteria, demonstrated with data showing potentiation of piperacillin against P. aeruginosa PA01 and E. coli 25922. With the CLSI inoculation of 3×10⁵ CFU/mL, 0.5 μg/mL (0.85 μM) of 600-Da BPEI lowered the piperacillin MIC from 4 to 1 μg/mL. However, 4.25 μM of PEG₃₅₀-BPEI₆₀₀ is required to lower the piperacillin MIC from 4 to 1 μg/mL. The inoculum effect on antibiotic MIC's is well known. When the inoculum is 5×10⁷ CFU/mL, the piperacillin MIC increases to 128 μg/mL and the MIC is reduced using a potentiator (Table 5). Potentiation against E. coli 25922 (3×10⁵ CFU/mL) requires 8 μg/mL (13.6 μM) of BPEI₆₀₀ to lower the piperacillin MIC from 1 to 0.125 μg/mL and 32 μg/mL (34 μM) of PEG₃₅₀-BPEI₆₀₀ to lower the piperacillin MIC from 1 μg/mL to 0.25 μg/mL.

Certain cationic compounds, such as aminoglycosides and polymyxins, have been reported as leading to nephrotoxicity. A presumption that all BPEIs are toxic overlooks the stipulation that toxicity depends on molecular weight and concentration. BPEI is available with a wide range of sizes (600 to 1,000,000 Da) and has been tested in a wide range of concentrations. High molecular weight BPEIs (over 25,000 Da) are toxic, whereas low MW BPEI (e.g., <25,000 Da) is not toxic unless their concentration is orders-of-magnitude higher than amount required for potentiation. Low cytotoxicity has been confirmed in our lab. Exposure to colon, kidney, HeLa cells leads to IC₅₀ values much higher than the concentration required for in vitro efficacy. The IC₅₀ values for BPEI₆₀₀ (300-1,000 μg/mL) are orders of magnitude higher than the amount required for potentiation (1-8 μg/mL). An in vitro nephrotoxicity assay was performed using primary human renal proximal tubule epithelial cells (hRPTECs). Exposure to BPEI₆₀₀ caused minimal release of LDH (3.5% at 62 μg/mL) and is lower than release values for cationic colistin (26% at 62 μg/mL). These data indicate low BPEI toxicity.

The reason for the low toxicity centers on its hydrophilic nature. BPEI₆₀₀ is miscible with water. Secondly, BPEI₆₀₀ does not contain regions of hydrophobic character, such as seen with cationic peptides, aminoglycosides, and polymyxins. Thus, BPEI₆₀₀ lacks the energetic force that drives hydrophobic compounds into lipid membranes. To the contrary, 25,000-1,000,000 Da BPEI possess hydrophobic interiors that increase lipophilicity and membrane penetration. Nevertheless, recognizing the need to alleviate safety concerns, we have undertaken PEGylation of low MW BPEI such as BPEI₆₀₀. Herein, we show that PEG-BPEI copolymers have lower in vivo toxicity than BPEI₆₀₀ alone. Over 3 days, female ICR mice were exposed daily to BPEI₆₀₀ and its modification with a single PEG₃₅₀ chain (PEG₃₅₀BPEI₆₀₀) or a single PEG₁₀₀₀ chain (PEG₁₀₀₀BPEI₆₀₀). For BPEI₆₀₀, the MTD (or LD₀) is 25 mg/kg but for PEG₃₅₀BPEI₆₀₀ the MTD is 75 mg/kg and for PEG₁₀₀₀PEI₆₀₀ the MTD is over 200 mg/kg (the highest amount tested).

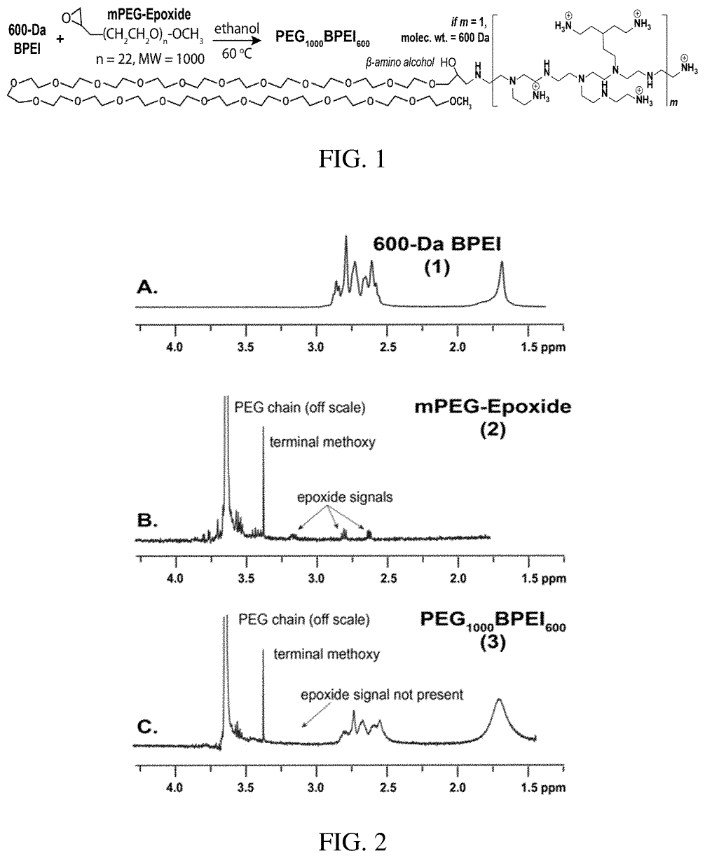

In one non-limiting embodiment, PEGylation of a low MW BPEI (e.g., BPEI₆₀₀) is performed with mPEG-Epoxide (e.g., 350 MW, 550 MW, 750 MW, 1000 MW, or 2000 MW) using a reaction scheme shown in FIG. 1. The reaction proceeds in anhydrous ethanol at 60° C., as shown in the NMR spectra (FIG. 2) where the epoxide-ring signals disappear. In non-limiting embodiments, the BPEI is decorated with the 350, 550, 750, 1000, or 2000 MW PEG separately in three different molar ratios of 1:1, 2:1, and 3:1 each.

Poly(ethylenimine)s can be readily modified by covalently attaching methyl end-capped polyethylene glycol chains (PEGs, CH₃[OCH₂CH₂]ₙ— with various n values) to its amine nitrogens (e.g. see FIG. 1). This can be accomplished by various means including nucleophilic substitution by the nitrogens of poly(ethylenimines) on PEGs with terminal leaving groups (such as halogens or sulfonates), by reductive amination on PEGs with carboxylic acid end-groups, and by conjugate addition of the nitrogens of poly(ethylenimines) to PEGs with acrylate end-groups. However, perhaps the most convenient strategy is to react the nitrogens of poly(ethylenimines) with PEGs having glycidyl epoxide end-groups to form β-aminoalcohol linkages (FIG. 1). PEG-type diepoxides react with ferrocenyl-modified poly(ethylenimine)s, even in aqueous media. It is generally reported that 1° amines are more reactive with glycidyl epoxides than 2° amines, and 3° amines are essentially non-reactive as they do not have protons to transfer. However, the reported selectivity's are modest, ranging from 1°/2° reactivity ratios of about 1.5/1 to about 16/1. Therefore, “PEGylation” of BPEIs will occur mostly on the 1° amines and reaction temperatures below 80° C. can be used to ensure 1° amines are the predominant reaction site. PEG-BPEI compositions having an approximate 1:1 ratio retains efficacy (Table 3) and has a higher maximum tolerable dose than BPEI₆₀₀ itself. The reaction can be performed in absolute ethanol and can be conveniently followed using ¹H-NMR spectroscopy by observing the characteristic epoxide signals disappear (FIG. 2). We can also draw on the other amine reactive PEGs, such as mPEG-Mesylate and mPEG-Tosylate.

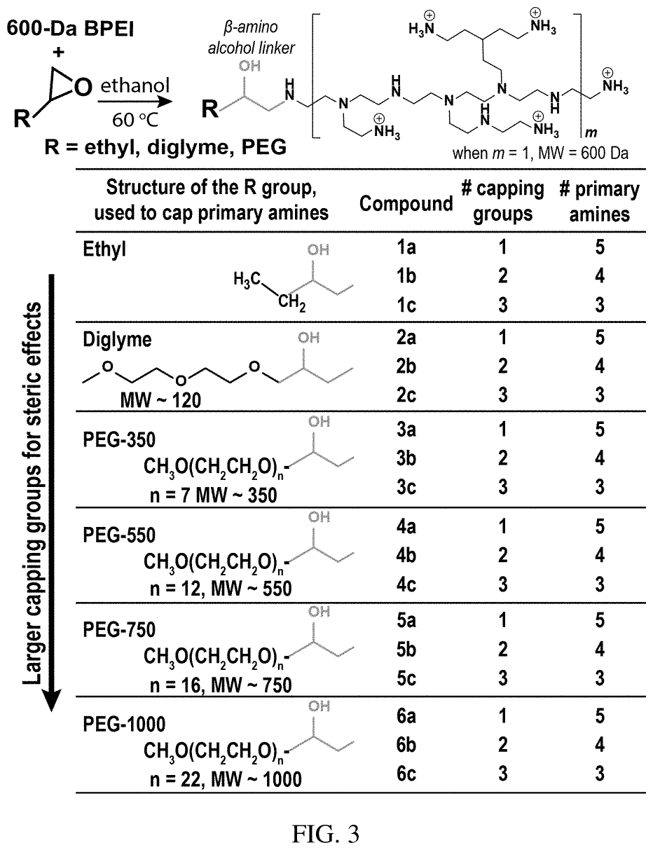

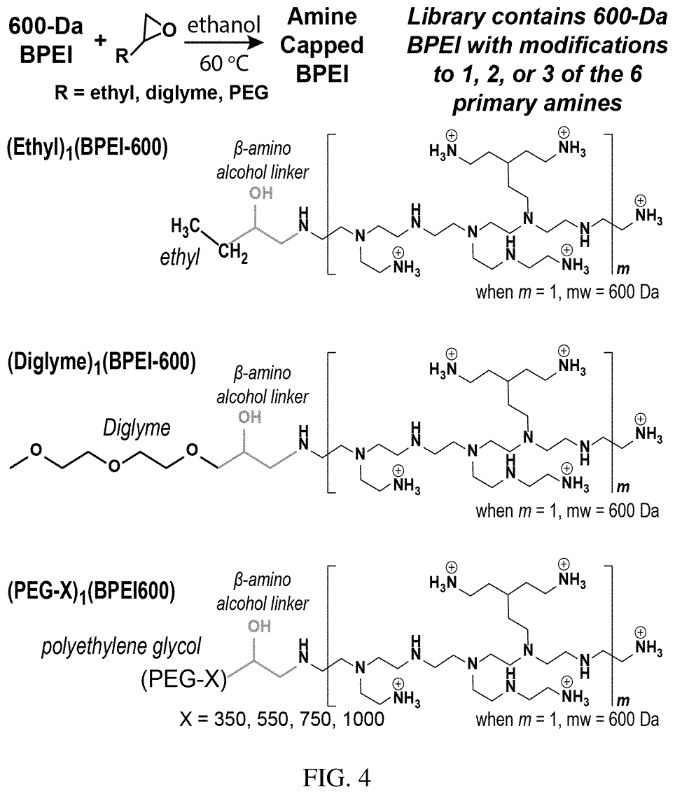

In one non-limiting embodiment, an exemplary library of cationic potentiators based on BPEI₆₀₀ with capped amines was created. In general, BPEI₆₀₀ is less toxic than 1200, 1800 or 10,000-Da BPEI, reducing the number of primary amines further reduces toxicity. In one embodiment epoxide ring-opening chemistry is used to react the primary amines of BPEI₆₀₀ with moieties having glycidyl epoxide end-groups to form β-amino alcohol linkages (FIGS. 3 and 4). Primary amines of BPEI₆₀₀ were reacted with an ethyl, diglyme, or PEG molecules having a glycidyl epoxide end-group. FIG. 3 shows the general reaction steps and FIG. 4 shows a list of the resulting compounds. The capping groups are hydrophilic but vary in steric bulk.

The single-step reaction occurs under mild conditions (ethanol solvent at 60° C.) with minimal workup (single pass through a silica gel column). The different potentiators balance cationic properties (for binding to the anionic targets) and reducing the number of amines with hydrophilic groups (to reduce toxicity). These criteria are met by capping amines with ethyl, diglyme, and PEG groups. Unlike rigid cyclic peptides, capped BPEIs are flexible structures that can access anionic sites on the flexible LPS and WTA molecules. Using TransPharm Preclinical Solutions Inc., the acute toxicity, or maximum tolerable dose (MTD), was evaluated over 3 days using female ICR mice with daily sc q24h dosing. For BPEI₆₀₀, the MTD is 25 mg/kg. Capping BPEI₆₀₀ with single PEG₃₅₀ chain (forming PEG₃₅₀-BPEI₆₀₀) increased MTD to 75 mg/kg but modification with a single PEG₁₀₀₀ chain (PEG₁₀₀₀-BPEI₆₀₀) increased MTD to over 200 mg/kg.

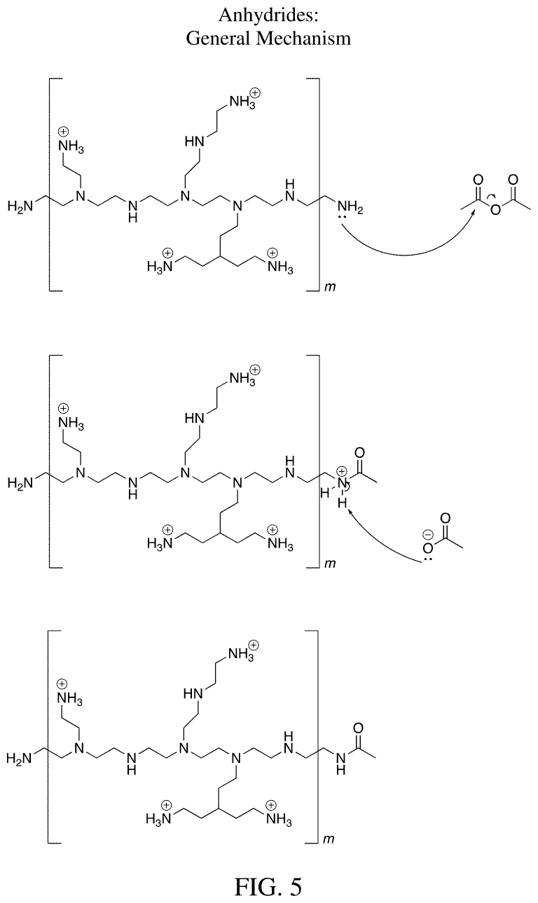

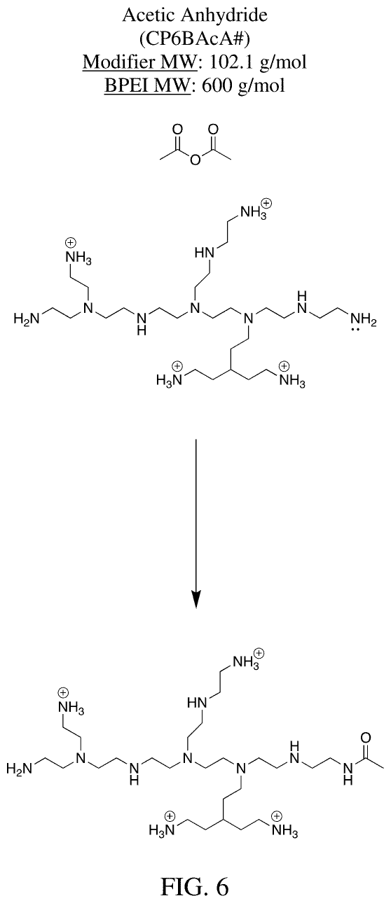

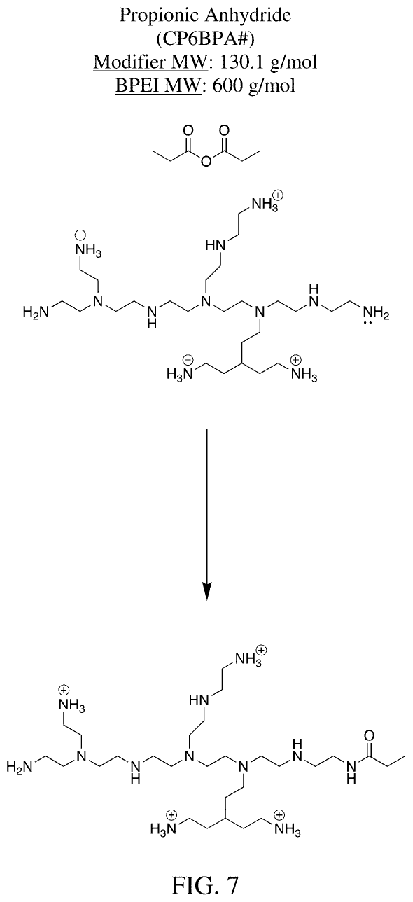

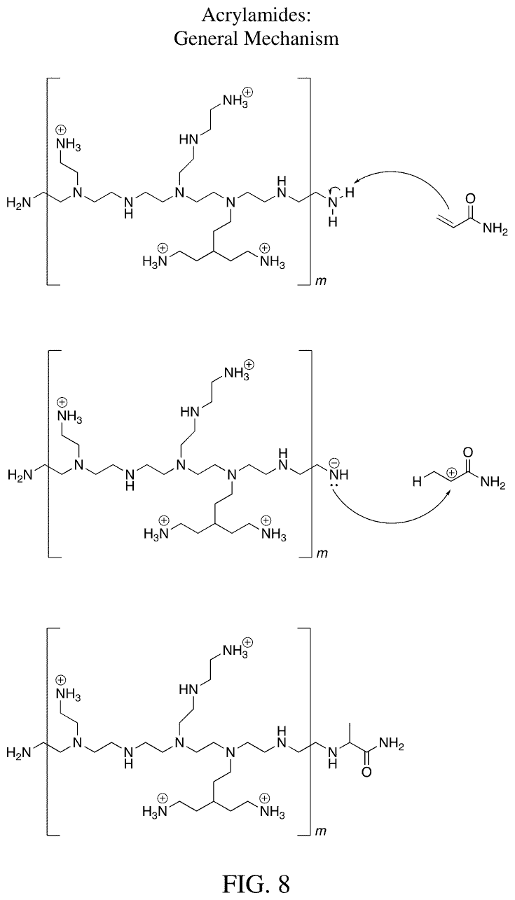

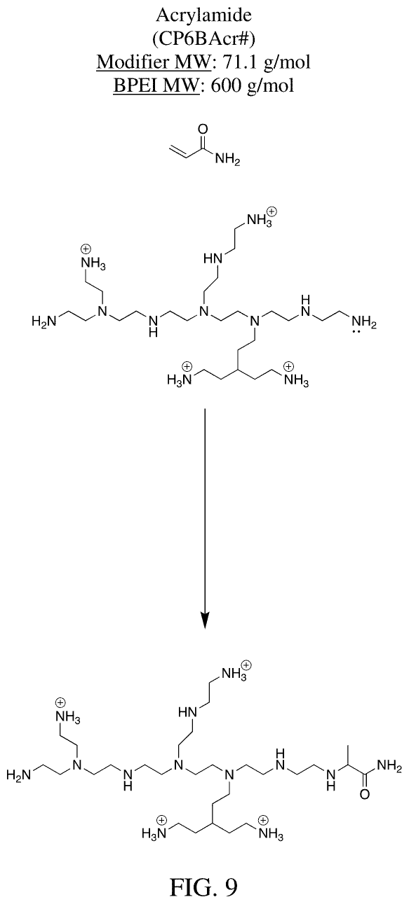

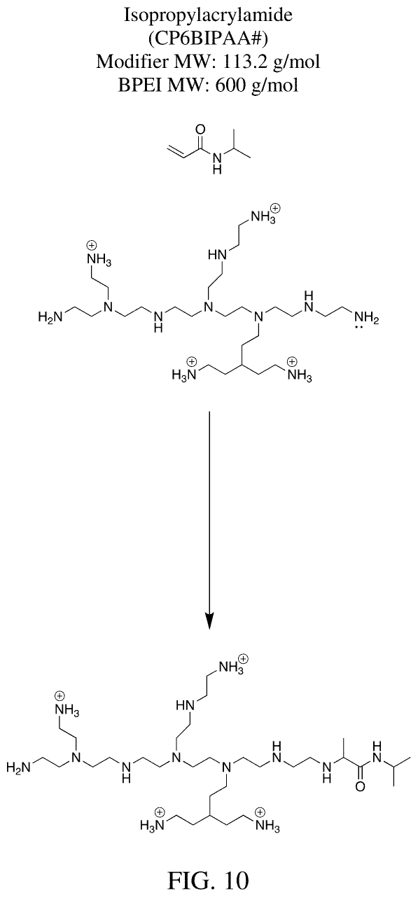

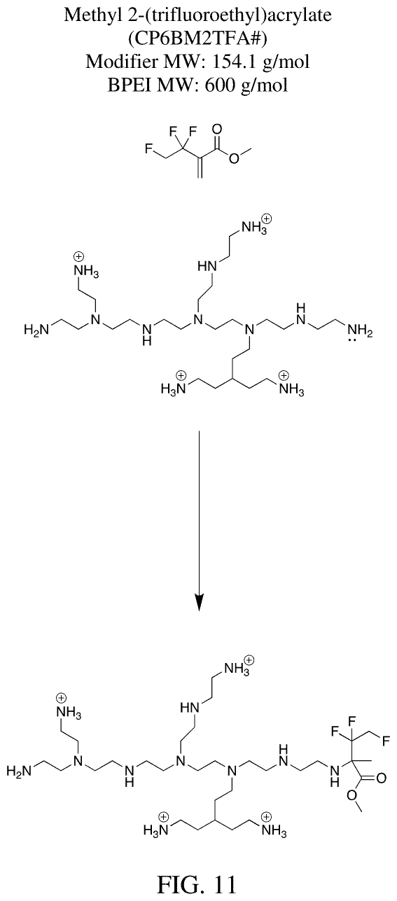

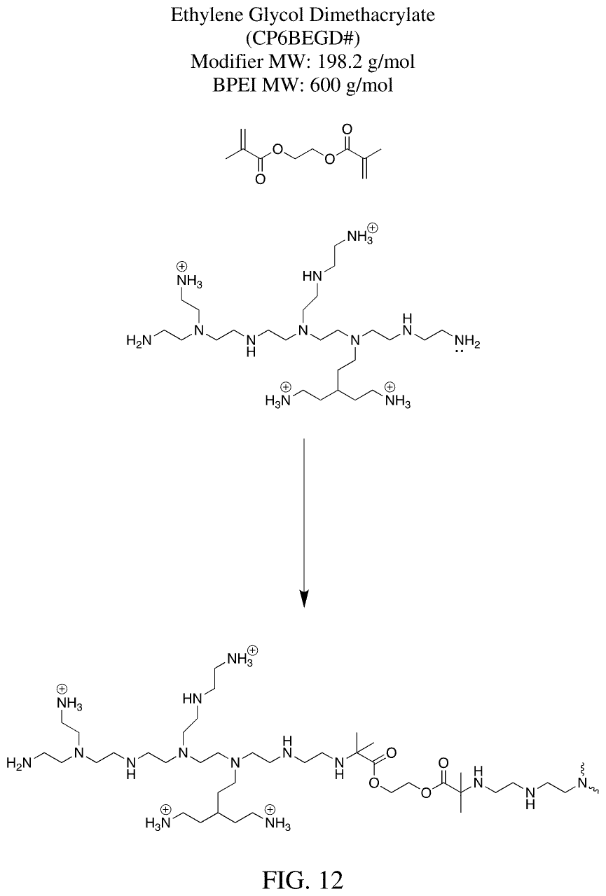

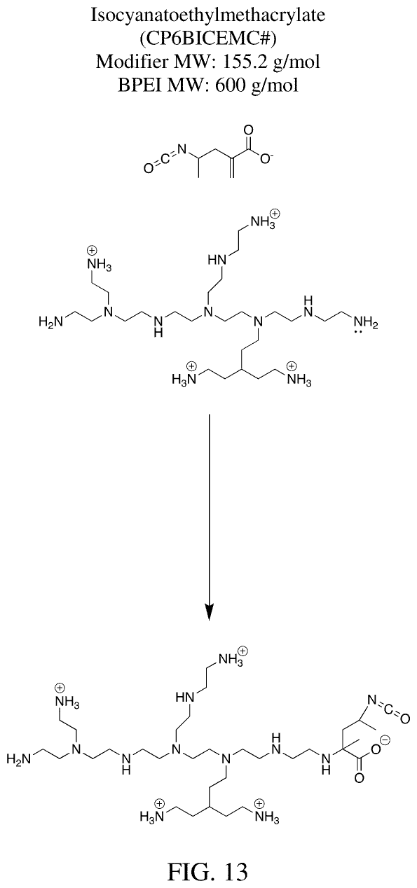

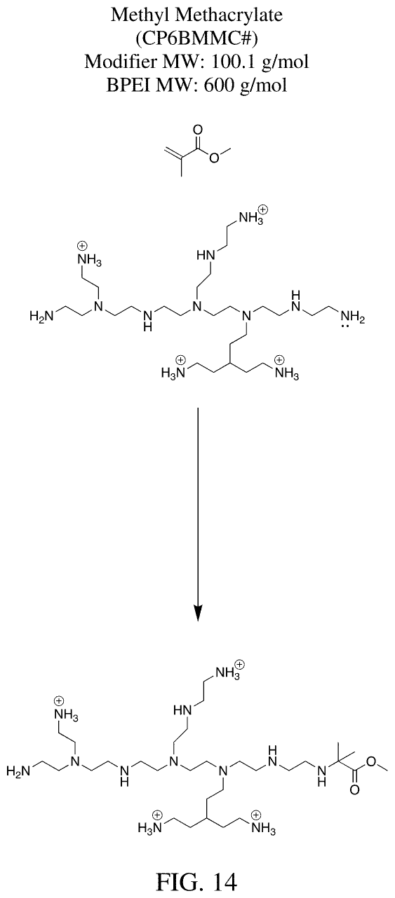

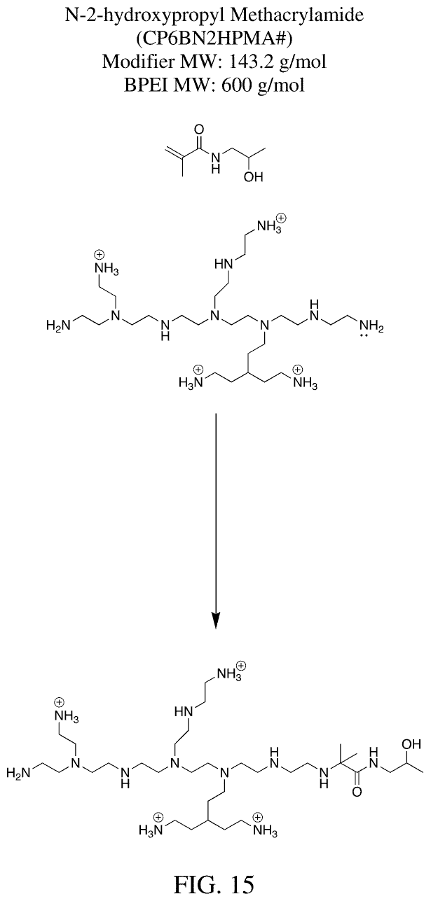

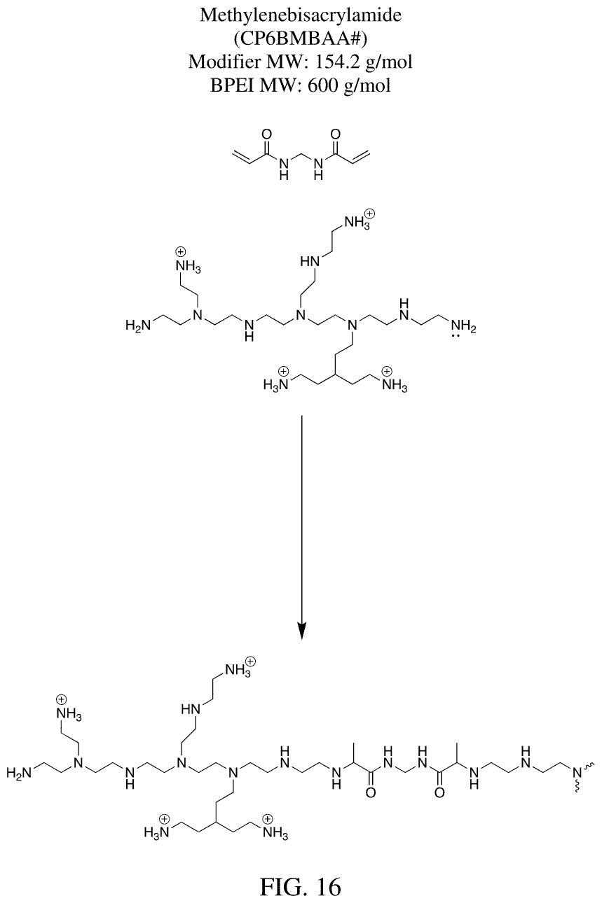

In addition to the PEGylation and epoxide reactions shown and discussed, the compositions and methods of the present disclosure can utilize BPEI molecules which have been modified to form other compounds, such as but not limited to anhydrides (e.g., FIGS. 5-7), acrylamides (e.g., FIGS. 8-10), acrylates (e.g., FIG. 11), methacrylates (e.g., FIGS. 12-14), methacrylamides (e.g., FIG. 15), and bis-methacrylates and bis-acrylamides (e.g., FIG. 16) to form bridged dimers, e.g., (BPEI)-linker-(BPEI).

TABLE 1
Disabling β-lactam Resistance in MRSA
strain USA300 with BPEI₆₀₀ (600-Da BPEI)

| | | |
|---|---|---|
| | | MIC with |
| MRSA | MIC of | 4 μg/ml |
| USA 300 | antibiotic | (6.75 μM) |
| ATCC 1717 | only | 600-Da BPEI |

| 600-Da BPEI | 64 | μg/ml | — | |
|---|---|---|---|---|
| oxacillin | 16 | μg/ml | 1 | μg/ml* |
| amoxicillin | 4 | μg/ml | 1 | μg/ml* |
| piperacillin | 4 | μg/ml | 1 | μg/ml* |
| cephalexin | 2 | μg/ml | 0.5 | μg/ml* |
| cefepime | 4 | μg/ml | 1 | μg/ml* |
| imipenem | 4 | μg/ml | 0.03 | μg/ml* |
| vancomycin | 1 | μg/ml | 1 | μg/ml |
| linezolid | 0.5 | μg/ml | 0.5 | μg/ml |

*below USCAST breakpoints

TABLE 2
Potentiation of Antibiotics against S. epidermidis
using BPEI₆₀₀ (600-Da BPEI)

| | | | antibiotic + | | antibiotic + | |
|---|---|---|---|---|---|---|
| | MIC of | | 2 μg/ml | | 4 μg/ml | |
| | antibiotic | | (3.375 μM) | | (6.75 μM) | |
| | only | | 600-Da BPEI | | 600-Da BPEI | |
| A MSSE 12228 | | | | | | |
| 600-Da BPEI | 16 | μg/ml | — | | — | |
| oxacillin | 0.1 | μg/ml | 0.1 | μg/ml | 0.1 | μg/ml |
| vancomycin | 1 | μg/ml | 1 | μg/ml | 1 | μg/ml |
| linezolid | 0.25 | μg/ml | 0.25 | μg/ml | 0.25 | μg/ml |
| B MRSE 35984 | | | | | | |
| 60a-Da BPEI | 16 | μg/ml | — | | — | |
| oxacillin | 64 | μg/ml | 2 | μg/ml | 0.25 | μg/ml |
| vancomycin | 2 | μg/ml | 2 | μg/ml | 0.5 | μg/ml |
| linezolid | 1 | μg/ml | 0.5 | μg/ml | 0.25 | μg/ml |

TABLE 3
Potentiation of Antibiotics against MRSA strain USA300 using
PEGylated BPEI (PEG₃₅₀- BPEI₆₀₀ or PEG₁₀₀₀-BPEI₆₀₀)

| | | antibiotic + | antibiotic + |
|---|---|---|---|
| A MRSA | MIC of | 8 μg/ml | 1 μg/ml |
| USA 300 | antibiotic | (8.5 μM) | (17 μM) |
| ATCC 1717 | only | PEG₃₅₀BPEI₆₀₀ | PEG₃₅₀BPEI₆₀₀ |
| PEG₃₅₀BPEI₆₀₀ | 64 μg/ml | — | — |
| oxacillin | 16 μg/ml | 8 μg/ml | 0.5 μg/ml |

| | | antibiotic + | antibiotic + |
|---|---|---|---|
| B MRSA | MIC of | 32 μg/ml | 64 μg/ml |
| USA 300 | antibiotic | (20 μM) | (40 μM) |
| ATCC 1717 | only | PEG₁₀₀₀BPEI₆₀₀ | PEG₁₀₀₀BPEI₆₀₀ |
| PEG₁₀₀₀BPEI₆₀₀ | 256 μg/ml | — | — |
| oxacillin | 16 μg/ml | 8 μg/ml | 0.5 μg/ml |
| ceftizoxime | 128 μg/ml | 8 μg/ml | 0.5 μg/ml |

TABLE 4
Disabling Resistance in MRSA strain USA300 using PEGylated
BPEI (PEG₃₅₀-BPEI₆₀₀ or PEG₁₀₀₀-BPEI₆₀₀)

| | | antibiotic + |
|---|---|---|
| MRSA | MIC of | 8 μg/ml |
| USA 300 | antibiotic | (8.5 μM) |
| ATCC 1717 | only | PEG₃₅₀-BPEI₆₀₀ |
| (PEG350)₁(BPEI600) | 64 μg/ml | — |
| oxacillin | 16 μg/ml | 1 μg/ml |
| | | antibiotic + |
| MRSA | MIC of | 32 μg/ml |
| USA 300 | antibiotic | (20 μM) |
| ATCC 1717 | only | PEG₁₀₀₀-BPEI₆₀₀ |
| (PEG1000)₁(BPEI600) | 256 μg/ml | — |
| oxacillin | 16 μg/ml | 1 μg/ml |

TABLE 5
Disabling Resistance in P. aeruginosa
PA01 with BPEI₆₀₀ and PEG₃₅₀-BPEI₆₀₀

| | | antibiotic + |
|---|---|---|
| P. aeruginosa | MIC of | 0.5 μg/ml |
| PA01 | antibiotic | (0.85 μM) |
| ATCC BAA-47 | only | 600-Da BPEI |
| 600-Da BPEI | 8 μg/ml* | — |
| piperacillin* | 4 μg/ml* | 1 μg/ml* |
| piperacillin** | 128 μg/ml** | 16 μg/ml** |

| | | antibiotic + |
|---|---|---|
| P. aeruginosa | MIC of | 4 μg/ml |
| PA01 | antibiotic | (4.25 μM) |
| ATCC BAA-47 | only | PEG₃₅₀BPEI₆₀₀ |
| (PEG350)₁(BPEI600) | 32 μg/ml* | — |
| piperacillin* | 4 μg/ml* | 1 μg/ml* |
| piperacillin** | 128 μg/ml** | 32 μg/ml** |

*standard CLSI inoculum, 3 × 10⁵ CFU/ml
**higher inoculum, 5 × 107 CFU/ml

### Example 2: Antibiofilm Synergistic Effects of β-Lactam/BPEI and β-Lactam/PEG-BPEI

Bacterial biofilms that are impenetrable to antibiotics pose an even greater threat when they are created by drug resistant bacteria. MRSA, MRSE, and their biofilms lead to chronic wound infections (i.e. wounds that have not proceeded through a reparative process in three months) that affect millions of Americans each year. With a dwindling arsenal of new antibiotics, existing drugs and regimens must be coupled with potentiators and re-evaluated as combination treatments for biofilms and antibiotic-resistant diseases.

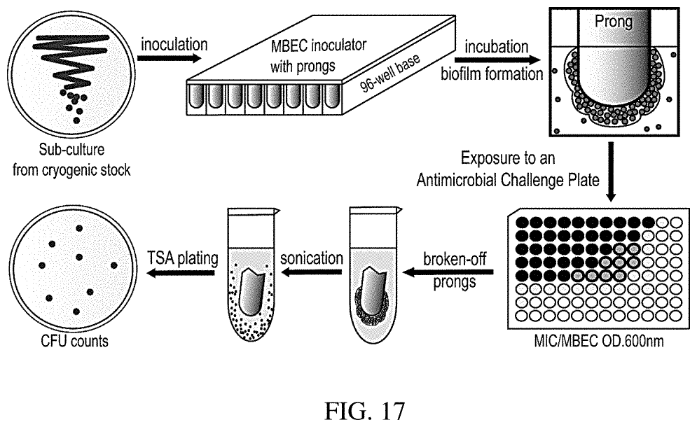

As noted above, BPEI successfully disabled resistance in MRSE strains, restoring their susceptibility to traditional β-lactam antibiotics. These formulations can also be applied to treating biofilms such as MRSE biofilms. The work below was conducted using the MBEC (Minimum Biofilm Eradication Concentration) assay, which is represented in FIG. 17 as a schematic flow. The results demonstrate antibiofilm activity in BPEI alone as well as synergistic effects between BPEI and β-lactams against MRSE biofilms. Since it can both disable resistance mechanisms and eradicate biofilms, BPEI is a dual-function potentiator, making it an ideal means of preventing and treating healthcare-associated S. epidermidis biofilms.

Table 6 below for example shows how BPEI compounds with antibiotic can disrupt biofilms and trigger biofilm death, for example the compounds disable resistance in MRSE from PBP2a and its biofilm. The MBEC was measured using the Calgary Biofilm Device, with plastic pegs on the plate lid and designed for robust and reproducible measurement of MBEC.

TABLE 6
Disabling Resistance from the -MecA gene, EPS
slime, and Biofilms in S. epidermidis

| | | antibiotic + |
|---|---|---|
| | MIC of | 4 μg/ml |
| A MRSE | antibiotic | (675 μM) |
| 35984 | only | 600-Da BPEI |
| 600-Da BPEI | 16 μg/ml | — |
| oxacillin | 64 μg/ml | 0.25 μg/ml |
| vancomycin | 2 μg/ml | 0.5 μg/ml |
| linezolid | 1 μg/ml | 0.25 μg/ml |

| | | antibiotic + |
|---|---|---|
| B MRSE | MBEC* of | 4 μg/ml |
| 35984 | antibiotic | (6.75 μM) |
| BIOFILM | only | 600-Da BPEI |
| 600-Da BPEI | 32* μg/ml | — |
| oxacillin | 128* μg/ml | 8* μg/ml |

*Minimum biofilm eradication concentration (MBEC)

Methods

Materials

In this work, the Staphylococcus epidermidis bacteria were purchased from the American Type Culture Collection (ATCC 29887: methicillin-resistant/biofilm-producer, ATCC 35984: methicillin-resistant/biofilm-producer, and ATCC 12228: methicillin-susceptible/non-biofilm producer). Chemicals were purchased from Sigma-Aldrich (DMSO, growth media, and electron microscopy fixatives). Antibiotics were purchased from Gold Biotechnology. BPEI₆₀₀ was purchased from Polysciences, Inc. MBEC™ Biofilm Inoculator with 96-well base plates were purchased from Innovotech, Inc.

MBEC Assay

Inoculation and Biofilm Formation

A sub-culture of MRSE was grown from the cryogenic stock on an agar plate overnight at 35° C. The MBEC plate was inoculated with 150 μL of TSB/well plus 1 μL of a stock culture made from 1 colony/mL of MRSE in TSB. The MBEC inoculator plate was sealed with Parafilm and incubated for 24 hours at 35° C. with 100 rpm shaking to facilitate biofilm formation on the prongs. Following biofilm formation, the lid of the MBEC inoculator was removed and placed in a rinse plate containing 200 μL of sterile PBS for 10 sec. Biofilm growth check (BGC) was performed by breaking a few prongs off using sterile pliers, submerging them in 1 mL PBS, and sonicating them on high for 30 minutes to dislodge the biofilm. After sonication, the biofilm solution was serial-diluted and spot-plated on agar plates for CFU counting to determine the biofilm density on the prongs.

Antimicrobial Challenge

A challenge plate was made in a new pre-sterilized 96-well plate in a checkerboard-assay pattern to test the synergistic activity of BPEI+antibiotic combinations. Antimicrobial solutions were serial-diluted and added to the 96-well plate, which contained 200 μL of cation-adjusted Mueller-Hinton broth (MHB) per well. Following the rinsing step and biofilm growth check, the MBEC inoculator lid was immediately transferred into the prepared antimicrobial challenge plate and incubated at 35° C. for 20-24 hours. After the challenge period, the MBEC inoculator lid was transferred into a recovery plate containing 200 μL of MHB per well, sonicated on high for 30 minutes to dislodge the biofilm and then incubated at 35° C. for 20-24 hour to allow the surviving bacterial cells to grow. After incubation, the OD₆₀₀ (optical density at 600 nm) of the recovery plate was measured using a Tecan Infinite M20 plate reader to determine the MBEC of the antimicrobial compounds tested. A change in OD₆₀₀ greater than 0.05 indicated positive growth. Likewise, the OD₆₀₀ for the base of the challenge plate was measured immediately after inoculation to determine the MICs of the antimicrobial compounds.

Scanning Electron Microscopy

MRSE 35984 cells were inoculated from 0.5% of an overnight culture and grown at 35° C. with shaking in the MBEC biofilm inoculator for 24 hours to facilitate biofilm formation on the prongs. Prongs were broken off the plate using a sterile plier, submerged, treated with primary fixative (5% glutaraldehyde in 0.1 M cacodylate buffer) in a capped vial, and incubated at 4±2° C. for 2 days. The prongs were removed from the fixing solution and air-dried for 72 hours in a fume hood. They were mounted on aluminum stubs with carbon tape and sputter-coated with AuPd. A Zeiss NEON SEM was used to image the samples at 5 kV accelerating voltage.

In a different experiment, MRSE 35984 cells were inoculated from 0.5% of an overnight culture and grown at 35° C. with shaking in the MBEC biofilm inoculator for 3 days to ensure maturation of biofilms on the prongs. Nutrient media was replaced every 24 hours. After 3 days, biofilms on the prongs were submerged into new 96-well base with BPEI (512 μg/mL) for 24 hours of treatment. Then, the prongs were broken off the plate using a sterile plier, submerged, fixed with primary fixative (5% glutaraldehyde in 0.1 M cacodylate buffer) in a capped vial, and incubated at 4±2° C. for 2 days. The prongs were removed from the fixing solution and air-dried for 72 hours in a fume hood. They were mounted on aluminum stubs with carbon tape and sputter-coated with AuPd. A Zeiss NEON SEM was used to image the samples at 5 kV accelerating voltage.

Biofilm Disrupting Assay

Two similar sets of the experiment were conducted: one used BPEI₆₀₀ and the other used BPEI_{10,000}. A sub-culture of MRSE 35984 was grown from the cryogenic stock on an agar plate overnight at 35° C. A pre-sterilized 96-well tissue-culture treated plate was inoculated with 100 μL of TSB/well plus 1 μL of a stock culture made from 1 colony/mL of MRSE in TSB. The plate was incubated at 35° C. for 24 hours to form mature biofilm. Planktonic bacteria were removed by washing 5 times with water. Crystal violet solution (0.1%) was used to stain the biofilm by adding 100 μL of the solution to each well for 15 minutes. The plate was then washed 5 times with water to remove all excess cells and dye. The plate was turned upside down and air-dried overnight.

Six separate treatments were performed on the preformed biofilm plate (total volume of 100 μL/well): untreated (negative control), BPEI-treated (32, 64, 128, and 256 μg/mL), and 30% acetic acid-treated (positive control). The treated samples were incubated at room temperature overnight to test the biofilm-disrupting ability of BPEI. Carefully, without touching the bottom of the plate, the solubilized solution in each well was transferred to a new flat-bottom plate for an absorbance measurement of OD₅₅₀. The OD₅₅₀ represents the amount of MRSE biofilm that was disrupted by BPEI, allowing for quantitative comparison of the controls and treated samples. Statistical data analysis among treated samples was performed using t-test, n=10.

Biofilm Kill Curve

Biofilm was grown in an MBEC inoculator plate for 24 hours with shaking to facilitate biofilm formation. At time zero, the prongs were sonicated in PBS for 30 minutes and then plated on agar for CFU counting. Four separate treatments were performed in a new 96-well base: Group 1 was the untreated control, Group 2 had 64 μg/mL of BPEI, Group 3 had 16 μg/mL of oxacillin, and Group 4 had a combination of 64 μg/mL of BPEI+16 μg/mL of oxacillin. The prongs on the MBEC inoculator were washed in PBS for 10 seconds and then transferred into the new treated base plate and incubated. Agar CFU plating was performed at 2 hours, 4 hours, 8 hours, and 24 hours for each treatment group. All the agar plating was incubated at 35° C. and counted for colony forming units the next day. Each trial was done in duplicate.

Results

During the staphylococcal biofilm attachment stage, bacteria adhere to a surface through non-covalent interactions (e.g. electrostatic bonds) via microbial surface components recognizing adhesive matrix molecules. The next stages are biofilm proliferation and maturation, during which EPS (containing proteins, polysaccharide intercellular adhesin PIA/PNAG, teichoic acids, and eDNA) and channel architecture are produced. During the last stage—biofilm detachment and dispersal—phenol soluble modulin peptides disrupt the non-covalent interactions established in the attachment stage. To survive in the human body, pathogens need to cope with the host defense mechanisms: the innate immune system, which includes neutrophils and antimicrobial peptides (AMPs) and the acquired immune system, which includes antigen-dependent T and B cells. The latter is ineffective against MRSE infections for reasons that are not well understood. Since they have been colonizing human skin for millennia, perhaps S. epidermidis strains have evolved ways to evade the host defenses. These recalcitrant biofilms particularly threaten immunocompromised patients and those who need prosthetic limbs or artificial implant devices because biofilms can survive on abiotic surfaces for weeks to months.

Confirmation of MRSE Biofilms

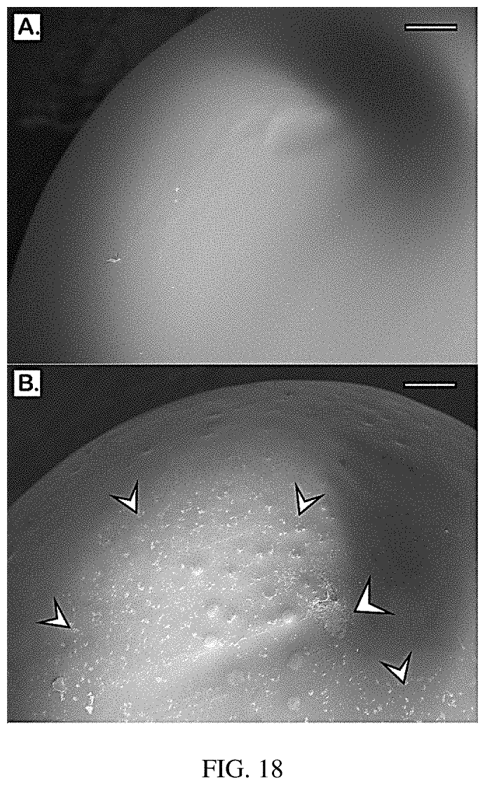

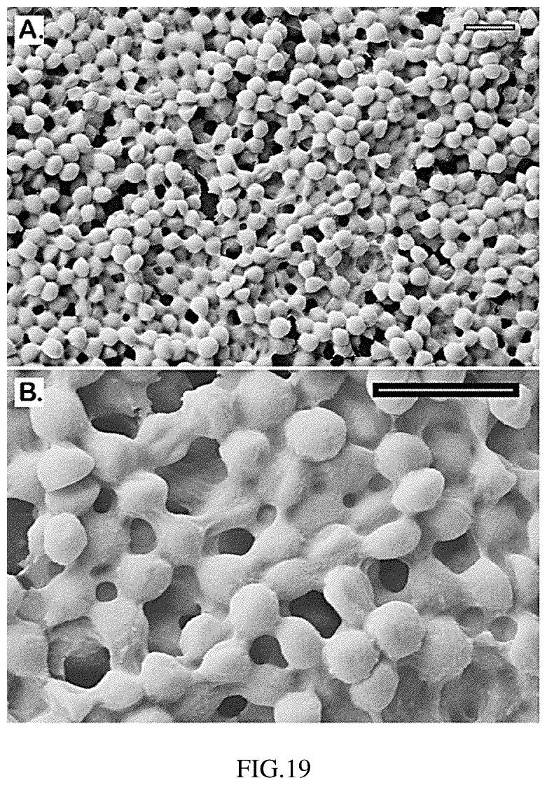

The MBEC plates with protruding-prong lids (FIG. 17) were used in our experiments to determine the antibiofilm activity of BPEI and conventional antibiotics. The prong lids with mature biofilms can fit into regular 96-well microtiter plates for further antimicrobial assays. Many biofilm studies fail to confirm biofilm presence before applying treatments. In this study, scanning electron microscopy (SEM) was performed to confirm that MRSE biofilms formed on the prongs after 24 hours inoculation. Compared to the smooth surface of the control prong (FIG. 18(a)), numerous microcolonies of MRSE were found on the inoculated prong (FIG. 18(b)), indicating that these prongs provide excellent surfaces for biofilm attachment and development. To better characterize the MRSE biofilm morphology, higher magnifications were obtained. Images depict spherical cocci of MRSE bacteria enfolded in a “blanket-like” coat of EPS matrix (FIG. 19(a)). The layers of bacteria are intertwined throughout the matrix, confirming the three-dimensional architecture and the existence of EPS in biofilms (FIG. 19(b)).

Among many substances in the EPS matrix, the poly-N-acetylglucosamine (PNAG, also known as PIA) polymer in particular was suggested to have a critical impact on S. epidermidis biofilms both in vitro and in vivo. Generated from the ica locus, this homopolymer is believed to interact with surface proteins and protect against host defense mechanisms during biofilm formation. Another important protective exopolymer is the pseudopeptide polymer poly-γ-DL-glutamic acid (PGA), which is encoded by the cap gene. Although PGA is produced in very small amounts, it plays a pivotal role in S. epidermidis resistance against host AMPs and leukocyte phagocytosis. These biopolymers, along with teichoic acids and eDNA, comprise the slime-like EPS coat. The SEM images confirms that mature MRSE biofilms have formed before treatment with BPEI and β-lactam combinations.

Efficacy of BPEI and β-Lactams Against MRSE Biofilms

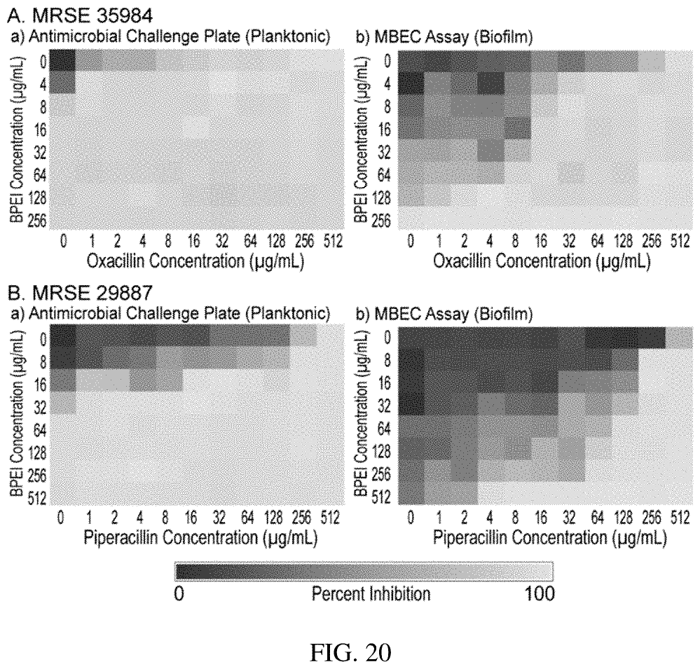

In S. aureus, we know that positively-charged BPEI electrostatically binds to negatively-charged wall teichoic acids. These interactions, which are also present in MRSE cell walls, disrupt the activity of penicillin-binding proteins PBP2a—an important resistance factor due to its low affinity for conventional β-lactam antibiotics—because WTA is essential for the full expression of oxacillin resistance from PBP2a. Thus, disabling PBP2a with 600-Da BPEI re-sensitizes MRSE to β-lactams. Here, we investigated a combination of BPEI and β-lactam antibiotics (oxacillin and piperacillin) against biofilms formed by two MRSE strains, MRSE ATCC 35984™ and MRSE ATCC 29887™. The MICs of BPEI and the antibiotics were found using the antimicrobial challenge plates, which measured the change in OD₆₀₀ of planktonic bacteria. Sonication of the prongs into a recovery plate allows us to measure the MBEC values, which were found to be much higher than the corresponding MIC values. This illustrates the intrinsic resistance of biofilms. For MRSE 35984, the oxacillin MIC is 16 μg/mL, while the oxacillin MBEC is 512 μg/mL (FIG. 20 (Aa)) Likewise, for BPEI, the MIC is 8 μg/mL whereas the MBEC is 256 μg/mL (FIG. 20(Ab)). With the addition of 8 μg/mL of BPEI, a synergistic effect lowered the MBEC of oxacillin from 512 to 32 μg/mL. Higher amounts of BPEI lowered oxacillin MBEC values further—for instance, 64 μg/mL of BPEI leads to an 8 μg/mL MBEC value for oxacillin. For MRSE 29887, the piperacillin MIC is 512 μg/mL, and the BPEI MIC is 64 μg/mL (FIG. 20(Ba)). Although the MBEC values for this strain were found to exceed 512 μg/mL (FIG. 20(Bb)), synergy between 64 μg/mL piperacillin and 128 μg/mL BPEI eradicated the biofilms.

BPEI can Disrupt Biofilms

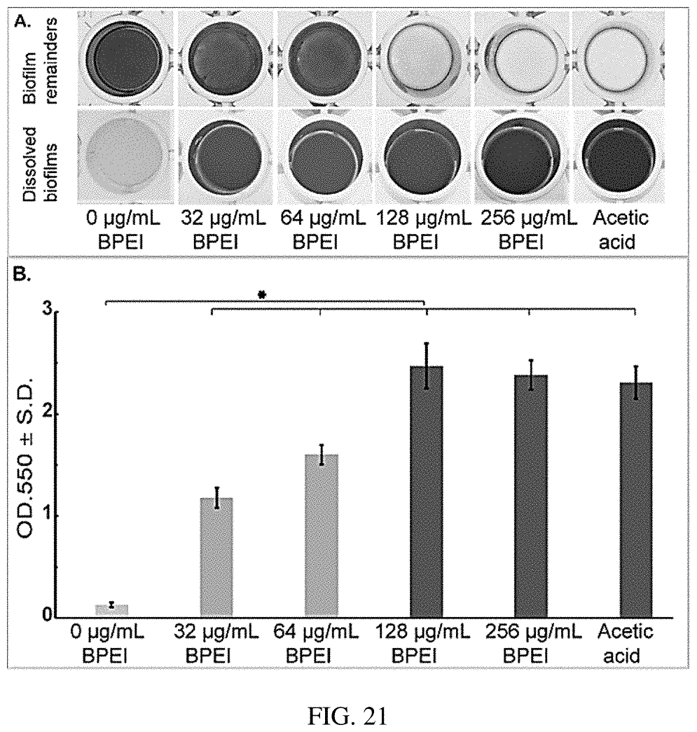

Staphylococcal AMP-defensive mechanisms involve the mprF gene, which modifies the phosphatidylglycerol with L-lysine as well as the D-alanylation of teichoic acids. Both processes lower the negative charge on the bacterial cell wall, thereby evading the cationic host AMPs. Without wishing to be bound by theory, our hypothesis is that cationic BPEI would have a similar electrostatic attraction to the bacterial cell wall, but the bacteria would not recognize BPEI as they would a host AMP and would therefore not deploy their defense mechanisms. Consequently, BPEI could partly neutralize the charge of the bacterial surface, thereby inhibiting biofilm formation and disrupting the EPS matrix so that antibiotics can enter and kill the bacteria. Our hypothesis was supported by the biofilm disrupting assay (FIG. 21). Mature biofilms of MRSE 35984 were stained with crystal violet and then treated with 32, 62, 128, and 256 μg/mL of BPEI₆₀₀. A negative control (0 μg/mL BPEI) and a positive control (acetic acid) were also performed. After 20 hours of treatment, BPEI-treated data was compared with the negative control using Student's t-test, and the results indicated that the MRSE biofilms were significantly dissolved by BPEI₆₀₀ (n=10, p-value<0.01). The dissolved biofilm solutions were carefully transferred to a new plate (without touching the bottom of the wells) for OD₅₅₀ measurement. As shown in FIG. 21(a), MRSE biofilm remained intact in the bottom of the negative control well, while the biofilm in the 32 and 64 μg/mL BPEI-treated wells were partially dissolved into solution. Biofilms treated with 128 and 256 μg/mL BPEI were completely dissolved, as was the biofilm treated with the positive control of acetic acid. FIG. 21(b) shows the OD₅₅₀ values of the crystal violet absorbance, which represent the amount of biofilm dissolved in each treatment.

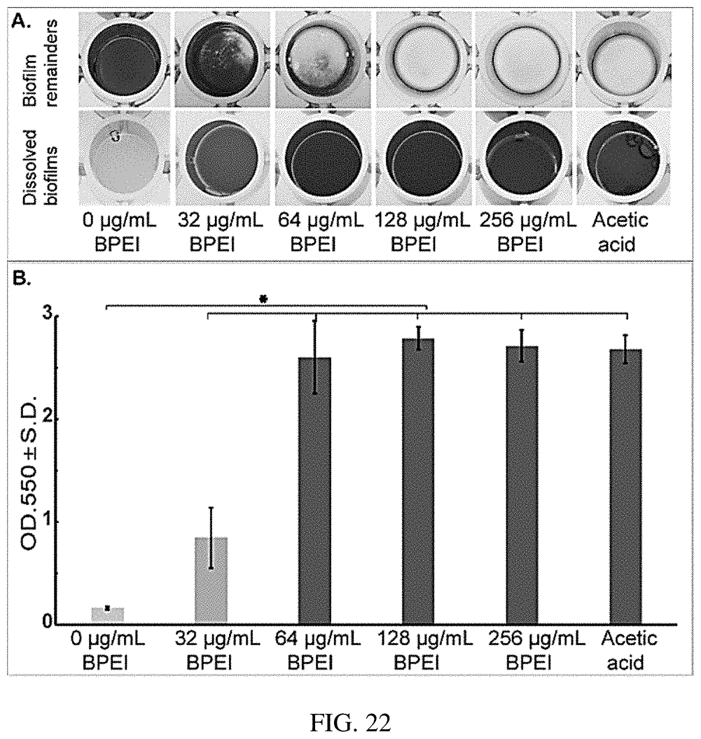

A similar experiment was conducted using BPEI_{10,000} (FIG. 22). As with BPEI₆₀₀, the t-test indicated that BPEI_{10,000} dissolved MRSE biofilms (n=10, p-value<0.01). Greater biofilm disruption effects were seen at 64 μg/mL of BPEI_{10,000}-treated samples (OD₅₅₀=2.60, FIG. 22(b)) than at 64 μg/mL of BPEI₆₀₀ treated samples (OD₅₅₀=1.59, FIG. 21(b)).

Biofilm Inhibition and Eradication Using Combination of BPEI+β-Lactams

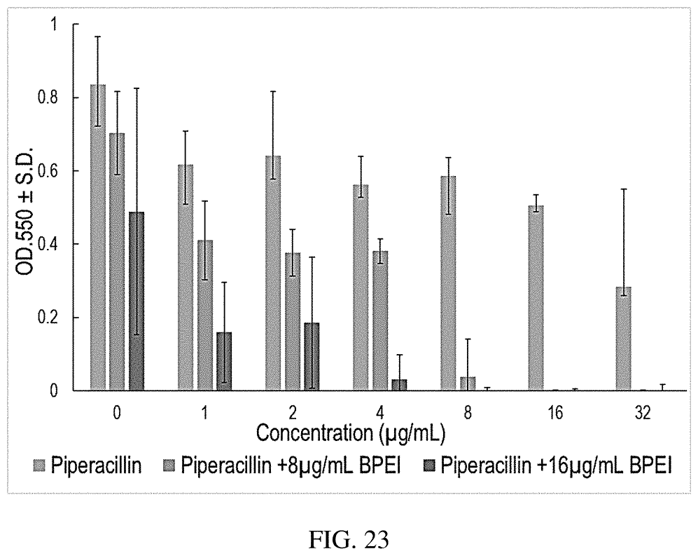

Crystal violet assays were used to demonstrate that BPEI synergizes with piperacillin to inhibit MRSE biofilm formation. Twenty-four hours after inoculation in a 96-well checkerboard plate containing combinations of BPEI₆₀₀ and piperacillin, the cell suspension supernatant was discarded, leaving the attached biofilms, which were then stained with crystal violet for measurement at OD₅₅₀ to quantify the remaining biomass. The Minimum Biofilm Inhibitory Concentration (MBIC) of BPEI was found to be 64 μg/mL, and the MBIC of piperacillin was 64 μg/mL. As shown in FIG. 23, less biofilm formed in BPEI+piperacillin combination wells than in the piperacillin wells. Additionally, higher concentrations of BPEI corresponded to greater inhibition of biofilm formation. For example, 8 μg/mL of BPEI and 16 μg/mL of piperacillin prevented biofilm growth, however 16 μg/mL of BPEI also prevented biofilm growth when combined with 8 μg/mL of piperacillin. These results confirm that BPEI₆₀₀ possesses inhibitory activity against MRSE biofilms.

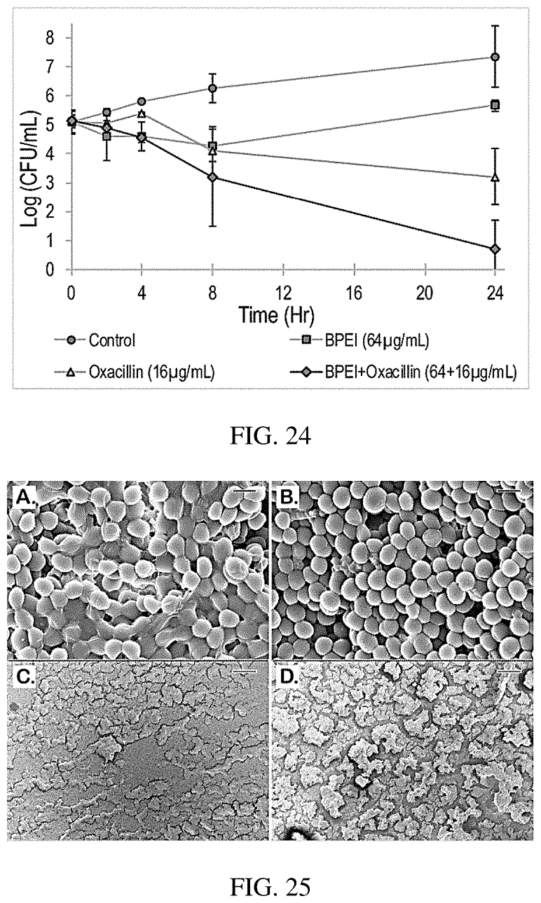

No antibiotic currently on the market can eradicate pathogenic biofilms, but our combination treatment can. To demonstrate this, mature biofilms of MRSE 35984 were treated in four different groups: Untreated control, BPEI-treated, oxacillin-treated, and combination (BPEI+oxacillin)-treated. A kill curve was generated to compare the antibiofilm activities of the treatments (FIG. 24). Before treatment, all four groups had the same cell density of approximately 10⁵ CFU/mL of bacteria. After treatments, the cell densities of each treated group were monitored by serial-diluting and agar-plating the sonicated prongs. Neither BPEI-treated nor oxacillin-treated groups could eradicate the biofilms, though they did inhibit the rate of the bacterial growth compared to the untreated control. At time 24 hours, cell densities were ˜10⁷ CFU/mL in the control group, ˜10⁵ CFU/mL in the BPEI-treated group, and ˜10³ CFU/mL in the oxacillin-treated groups. Since implantable medical devices have ample surface area for bacterial colonization, even a low bacterial inoculum (˜10² CFU/mL S. aureus) can provoke an infection. Oxacillin did eradicate some biofilm—as indicated by its declining kill curve in FIG. 24, but the remaining persister biofilm on the treated prongs (>10³ CFU/mL at 24 hours) are sufficient to grow and spread to new niches. Compared to the control group at time 24 hours (˜10⁷ CFU/mL), the combination treatment of BPEI+oxacillin reduced the cell density of the biofilms by 100,000-fold (<10¹ CFU/mL), illustrating the combination's synergistic ability to eradicate biofilms.

Efficacy of BPEI on 3-Day-Old Biofilms

To test our technology against a chronic wound model, we investigated BPEI's effects on a 3-day-old MRSE biofilm. MRSE 35984 was grown on the MBEC device for 3 days prior to treatment. Then, the untreated control and the BPEI-treated (512 μg/mL) samples were fixed and imaged for microscopic analysis. As shown in FIG. 25, the untreated MRSE biofilms were thick, and encased in EPS (FIG. 25(a)), and they densely occupied the entire prong surface (FIG. 25(c)). In contrast, after BPEI treatment, the EPS coat was visibly disrupted which reveal the bacterial cells with a thin or non-existent EPS coating (FIG. 25(b)), and a greater proportion of the prong surface was exposed (FIG. 25(d)). These results demonstrate that BPEI not only can effectively potentiate antibiotics against planktonic cells, but also against the stubborn mature biofilms through an EPS-disruption mechanism. The exposure of the individual cells without the EPS protection would make them more vulnerable to antimicrobial agents, increasing the likelihood of clinical treatment success against persistent pathogenic biofilms.

### Example 3: Effects of PEGylation on Toxicity of BPEI

As noted above, The persistence of MRSA, MRSE, and/or MDR-PA often allows acute infections to become chronic wound infections. The water-soluble hydrophilic properties of low molecular weight BPEI (e.g., BPEI₆₀₀) enable easy drug delivery to directly attack AMR and biofilms in the wound environment as a topical agent for wound treatment. To mitigate toxicity issues, we modified BPEI₆₀₀ with PEG in a one-step reaction. The PEG-BPEI molecules disable β-lactam resistance in MRSA, MRSE, and MDR-PA while also having the ability to dissolve established biofilms. PEG-BPEI accomplishes these tasks independently, resulting in a multi-function potentiation agent. In non-limiting embodiments of the disclosure, wounds can be treated with antibiotics given topically, orally, or intravenously in which external application of PEG-BPEIs disables biofilms and resistance mechanisms. In the absence of a robust pipeline of new drugs, existing drugs and regimens must be re-evaluated as combination(s) with potentiators. The PEGylation of BPEI₆₀₀ provides new opportunities to meet this goal with a single compound whose multi-function properties are retained while lowering acute toxicity.

Materials and Methods

Methicillin-resistant Staphylococcus epidermidis 35984™, methicillin-resistant Staphylococcus aureus USA300 (BAA-1717), and Pseudomonas aeruginosa 27853™ bacteria were purchased from the American Type Culture Collection. Additionally, MDR-PA OU1 was obtained from clinical isolates from the University of Oklahoma Health Sciences Center using appropriate IRB protocols and procedures. MRSA MW2 was a generous gift from Dr. Suzanne Walker. Chemicals and antibiotics were purchased from Sigma-Aldrich. BPEI₆₀₀ was purchased from Polysciences, Inc. Monofunctionalized PEG-epoxide was obtained from Nanocs, Inc.

Synthesis and Characterization of PEG₃₅₀-BPEI₆₀₀ Conjugate

Approximately 200 mg of BPEI₆₀₀ was added to a small glass vial and dried overnight under high vacuum. The vial was reweighed to determine the final mass of the dry BPEI. This value was used to determine the amount of mPEG-epoxide (350 MW) required to react with BPEI₆₀₀ in a 1-to-1 stoichiometric ratio. The BPEI₆₀₀ was dissolved in 3 mL of 100% ethanol with stirring. Afterwards, a solution of mPEG-epoxide dissolved in 3 mL of 100% ethanol was added dropwise. The mixture was stirred at 60° C. for 24 hours. Afterwards, the mixture was cooled, and solvent removed under high vacuum for 72 hours. A 1-D ¹H NMR spectrum was collected by dissolving a portion of the dry reaction product in CDCl₃ followed by transfer to a 3-mm NMR tube. All NMR experiments were performed using a 28-shim Agilent VNMRS-300 MHz equipped with a triple-resonance PFG probe. Pulse sequences for each experiment were supplied by Agilent. Data acquisition and processing were completed using VNMRJ 2.2C software on the Red Hat Linux 4.03 operating system. MestreNova software was used to analyze the spectra.

Checkerboard Assays

Checkerboard assays were used to determine the synergistic effect between PEG₃₅₀-BPEI₆₀₀ and antibiotics against drug resistant strains growing in cation-adjusted Mueller-Hinton broth (CAMHB). Bacterial growth used CAMHB media augmented with various amounts in serial dilutions of PEG₃₅₀-BPEI₆₀₀ and/or antibiotic (oxacillin or piperacillin) inoculated with bacterial cells from an overnight culture (5×10⁵ CFU/mL). Cells were grown at 37° C. The change in OD₆₀₀ (optical density at 600 nm) was measured and recorded after 24 hr of treatment. Each checkerboard trial was done in triplicate using sterile Greiner CellStar™ flat bottom polystyrene plates, catalog #655180.

In Vivo Toxicity Studies

Experiments to determine the acute toxicity of BPEI₆₀₀ and PEG₃₅₀-BPEI₆₀₀ were performed by a contract research organization (TransPharm Preclinical Solutions, Jackson, Mich.). Fully immunocompetent, uninfected, ICR mice (4-6 weeks old, 18-20 grams each, Envigo, Inc.) were treated once a day for 3 days via subcutaneous injection with low concentrations of BPEI₆₀₀ or PEG₃₅₀-BPEI₆₀₀ and closely monitored for adverse reactions. Adverse events and mortality were tracked through study Day 4. Mice were administered 6.25, 12.5, 25, 50, 75, and 100 mg/kg of BPEI₆₀₀ or PEG₃₅₀-BPEI₆₀₀ once daily on Day 0, 1 and 2 in a volume of 0.2 mL via subcutaneous (sc) injection, beginning with the lowest dose concentration before dosing the next highest concentration. Mice in each group were closely observed for 15 minutes following dose administration for adverse events prior to dosing the next highest dose concentration. Both BPEI₆₀₀ and PEG₃₅₀-BPEI₆₀₀ are very soluble in water, which was formulated in phosphate buffered saline (PBS) at 20 mg/mL solution and handled in a manner to minimize endotoxin and bacterial contamination. The solutions were sterilized by filter sterilization prior to the initial dose. The mice could tolerate 25 mg/kg of BPEI₆₀₀ and 75 mg/kg of PEG₃₅₀-BPEI₆₀₀ with no visible toxicity. Mice injected with 50 mg/kg of BPEI₆₀₀ and 100 mg/kg of PEG₃₅₀-BPEI₆₀₀ succumbed to death within 5 min of treatment.

Biofilm Disrupting Assay

Overnight cultures of MRSE 35984 were used to inoculate a tissue-culture treated 96-well plate (100 μL of tryptic soy broth or TSB/well) with an inoculation size of 1 μL/well (˜5×10⁵ CFU/mL). The plate was incubated at 35° C. for 48 hr to allow the bacteria to form biofilm. It was then washed with water to remove planktonic bacteria and stained with 100 μL of crystal violet solution (0.1%) per well for 15 min. The stained plate was washed excessively with water 5 times to remove any unbound stain and air-dried overnight. After washing to remove crystal violet, various concentrations of PEG₃₅₀-BPEI₆₀₀ or BPEI₆₀₀ were added to the stained-biofilm plate with a total volume of 100 μL/well. Water only and 30% acetic acid were also used for treatment. After 20 hr, without touching the biofilm layer in the bottom of the plate, solubilized solution containing dissolved, stained, biomass in each treated well was carefully transferred to a new 96-well plate for an OD₅₅₀ measurement, which represents the corresponding amount of biomass disrupted by each treatment.

Isothermal Titration Calorimetry (ITC)

Isothermal titration calorimetry (MicroCal PEAQ-ITC, Malvern Inc., Malvern, U.K.) was utilized to test the interactions between P. aeruginosa isolated LPS and PEGylated BPEI. Briefly, solutions of PEG₃₅₀-BPEI₆₀₀ (1 mg/mL) and P. aeruginosa LPS (Sigma product L8643, 5 mg/mL) prepared in 50 mM Tris-HCl (pH 7) were titrated using injections of 2 μL lasting 4 s and separated by 150 s time intervals. Controls were performed and the experiment was done in duplicate.

Results

The acute toxicity LD₀, or maximum tolerable dose (MTD) of BPEI₆₀₀ or PEG₃₅₀-BPEI₆₀₀ in LCR mice was evaluated over 3 days using female ICR mice with daily subcutaneous dosing studied. Toxicity data was collected by TransPharm Preclinical Solutions Inc., a contract research lab. For BPEI₆₀₀, the MTD is 25 mg/kg (Tables 7A-7B). Adding a 350 MW PEG (i.e., PEG₃₅₀) group to BPEI₆₀₀ (forming PEG₃₅₀-BPEI₆₀₀) increases the MTD (i.e., reduces the toxicity) of BPEI₆₀₀ to 75 mg/kg (Tables 8A-8B). These data show that subcutaneous PEG₃₅₀-BPEI₆₀₀, for instance applied to a wound with exposed tissue layers, has lower acute toxicity and is safer to use than BPEI₆₀₀. As described elsewhere herein, (PEG₃₅₀)-(BPEI₆₀₀) is a multi-functional broad-spectrum antibiotic potentiator that also disrupts biofilms. Moreover, Secondly, our potentiators are not cationic peptides nor peptide mimetics that can disable biofilms but lack in vivo activity due to rapid proteolytic degradation and/or protein binding in wounds.

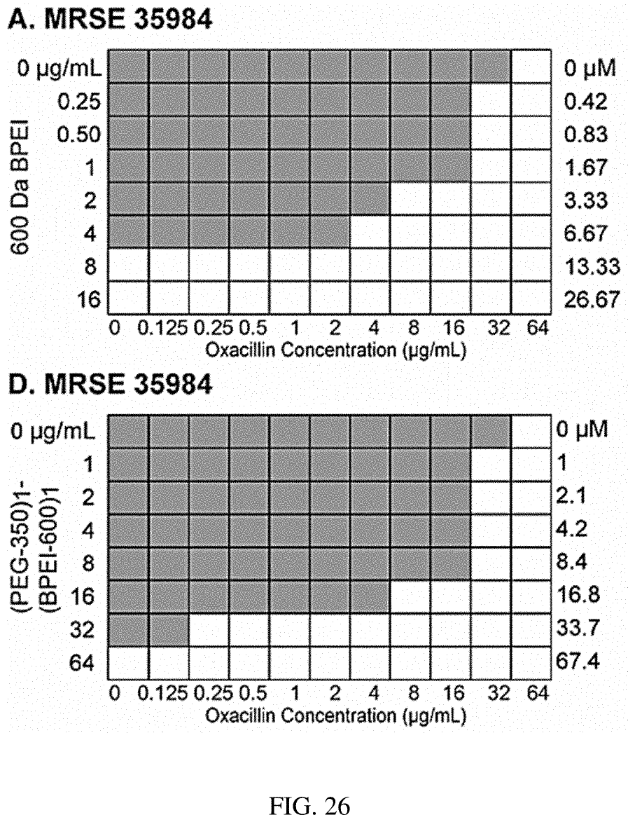

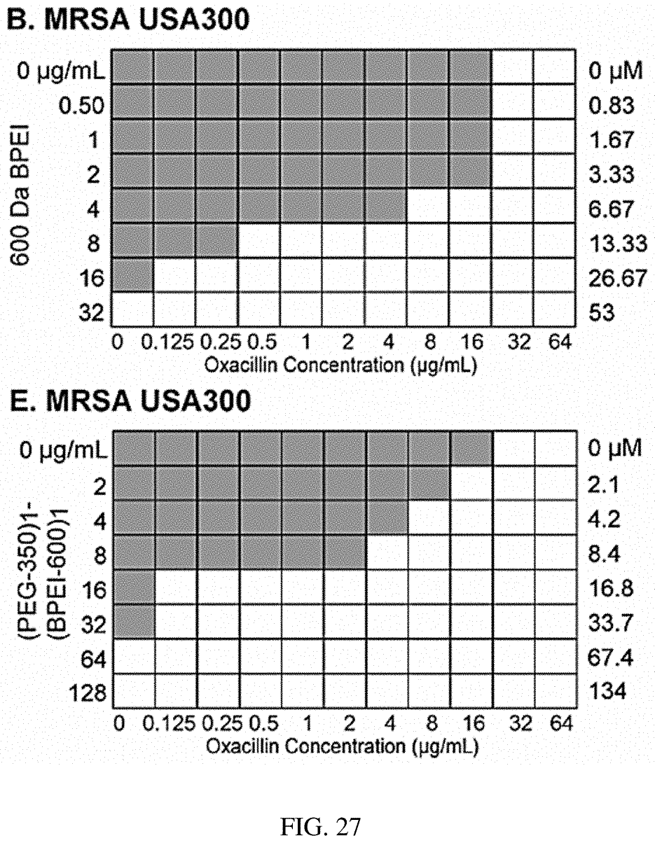

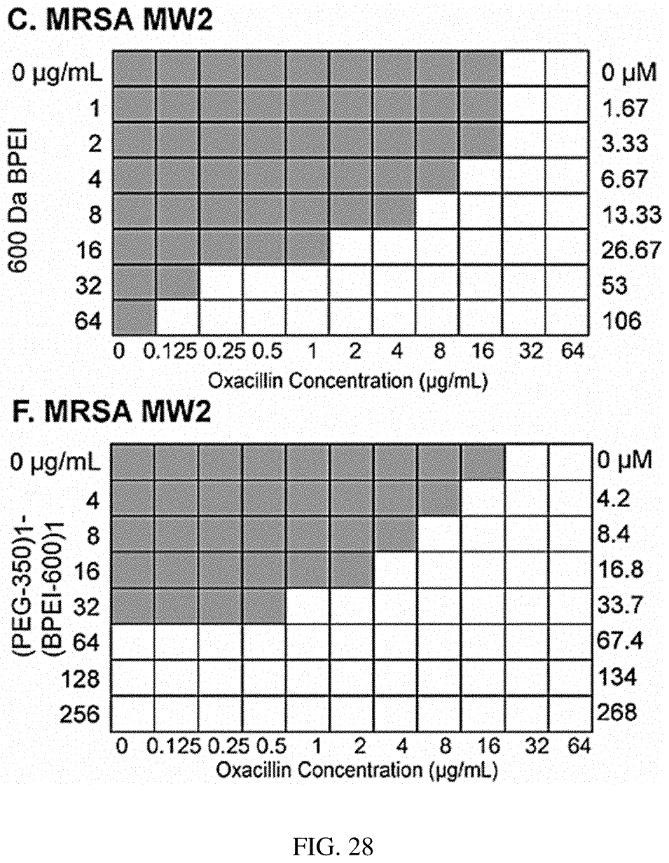

As shown above in Example 1, BPEI₆₀₀ restores susceptibility of MRSE and MRSA to β-lactam antibiotics. In this example, checkerboard assays were conducted to examine the potentiation activity of PEG₃₅₀-BPEI₆₀₀ against MRSE and MRSA when combined with oxacillin (FIGS. 26-27). The minimum inhibitory concentrations (MICs) of PEG₃₅₀-BPEI₆₀₀ and oxacillin for all three tested strains are tabulated in Table 9. The PEG₃₅₀-BPEI₆₀₀ MICs for MRSE 35984, MRSA MW2, and MRSA USA300 are each 64 μg/mL. The oxacillin MICs are 64 μg/mL for MRSE 35984, 32 μg/mL for MRSA MW2, and 32 μg/mL for MRSA USA300. According to standard EUCAST guidelines (European Committee for Antimicrobial Susceptibility Testing of the European Society of Clinical, M.; Infectious, D., EUCAST Definitive Document E.Def 1.2, May 2000: Terminology relating to methods for the determination of susceptibility of bacteria to antimicrobial agents. Clin Microbiol Infect 2000, 6 (9), 503-508), these values denote oxacillin resistance, the breakpoint MIC for resistance is ≥4 μg/mL while values<2 μg/mL denote oxacillin susceptibility. The checkerboard assay data show that growth inhibition is possible with different combinations of BPEI₆₀₀ and oxacillin, or PEG₃₅₀-BPEI₆₀₀ and oxacillin (FIGS. 26-28). Also according to the standard EUCAST guidelines, synergistic effects are indicated when the fractional inhibitory concentration index (FICI)≤0.5, which was found for all three strains tested. Non-PEGylated BPEI was slightly more effective than PEGylated BPEI₆₀₀ in overcoming oxacillin resistance. Achieving an oxacillin MIC of 2 μg/mL against MRSE 35984 required 3.3 μM of BPEI₆₀₀ vs. 33.7 μM of PEG₃₅₀-BPEI₆₀₀; 13.33 μM of BPEI₆₀₀ vs. 16.8 μM of PEG₃₅₀-BPEI₆₀₀ for MRSA USA300; and 26.67 μM of BPEI₆₀₀ vs. 33.7 μM of PEG₃₅₀-BPEI₆₀₀ for MRSA MW2. The ability to increase antibiotic efficacy can be described by a 4-fold Minimum Potentiating Concentration (MPC₄) (see Haynes, K. M.; Abdali, N.; Jhawar, V.; Zgurskaya, H. I.; Parks, J. M.; Green, A. T.; Baudry, J.; Rybenkov, V. V.; Smith, J. C.; Walker, J. K., Identification and Structure—Activity Relationships of Novel Compounds that Potentiate the Activities of Antibiotics in Escherichia coli. Journal of medicinal chemistry 2017, 60 (14), 6205-6219). The MPC₄-OXA for BPEI₆₀₀ was 3.33 μM for MRSE 35984, 6.67 μM for MRSA USA300, and 13.33 μM for MRSA MW2. For PEG₃₅₀BPEI₆₀₀, the MPC₄-OXA was 16.8 μM, 4.2 μM, and 8.4 μM for these three species, respectively. Without wishing to be bound by theory, the differences between PEGylated BPEI₆₀₀ and non-PEGylated BPEI₆₀₀ are likely caused by reducing the number of primary amines in BPEI₆₀₀ by PEGylation and/or steric effects of the PEG group. The methicillin resistance gene mecA is responsible for synthesis of PBP2a, a 78 kDa transmembrane protein that can block all bindings to β-lactams, enabling MRSA/MRSE to survive in the presence of these antibiotics. Wall teichoic acid (WTA) is known to be PBP2a's cofactor, which localizes PBP2a to where to function. As with BPEI₆₀₀, PEG₃₅₀-BPEI₆₀₀ bears positive charges from the amine groups at physiological pH, allowing it to electrostatically bind negatively charged phosphodiester backbone of WTA. Therefore, PEG₃₅₀-BPEI₆₀₀ likely inhibits proper localization of PBP2a/4, disabling this resistance factor and restoring susceptibility of MRSA and MRSE to β-lactams.

TABLE 9
Minimum inhibitory concentrations (MIC) and fractional inhibitory
concentration indices (FICI) of BPEI₆₀₀ and PEG₃₅₀-BPEI₆₀₀ as
potentiators of β-lactam activity against MRSA, MRSE, and P. aeruginosa.
Concentrations are listed in units of μg/mL and the corresponding μM values
are in parentheses for comparison between BPEI₆₀₀ and PEG₃₅₀-BPEI₆₀₀.

| | MIC μg/mL (μM) | | | | | | |
|---|---|---|---|---|---|---|---|
| | | | | OXA^{a} + | | | |
| Strain | 600 Da BPEI | | OXA^{a} | 600 Da BPEI | | FICI | Outcome |
| MRSE | 8 | (13.3) | 32 | 8 + 2 | (3.3) | 0.5 | Synergy |
| 35984 | | | | | | | |
| MRSA | 32 | (53.3) | 32 | 4 + 8 | (13.3) | 0.38 | Synergy |
| USA300 | | | | | | | |
| MRSA | >64 | (>106.7) | 32 | 2 + 16 | (26.7) | 0.19 | Synergy |
| MW2 | | | | | | | |

| | | | OXA^{a} + | | | |
|---|---|---|---|---|---|---|
| Strain | PEG-BPEI^{b} | OXA^{a} | PEG-BPEI^{b} | | FICI | Outcome |
| MRSE | 64 (67.4) | 64 | 8 + 16 | (16.7) | 0.38 | Synergy |
| 35984 | | | | | | |
| MRSA | 64 (67.4) | 16 | 2 + 16 | (16.8) | 0.38 | Synergy |
| USA300 | | | | | | |
| MRSA | 64 (67.4) | 16 | 4 + 16 | (16.7) | 0.5 | Synergy |
| MW2 | | | | | | |

| | | | PIP^{c,d} + | | | |
|---|---|---|---|---|---|---|
| Strain | 600 Da BPEI | PIP^{c} | 600 Da BPEI | | FICI | Outcome |
| PA 27583 | 16 (26.7) | 4 | 0.25 + 4 | (6.7) | 0.31 | Synergy |
| PA OU1 | 16 (26.7) | 64 | 4 + 2 | (3.3) | 0.31 | Synergy |

| | | | PIP^{c,d} + | | |
|---|---|---|---|---|---|
| Strain | PEG-BPEI^{b} | PIP^{c} | PEG-BPEI^{b} | FICI | Outcome |
| PA 27583 | 64 (67) | 4 | 0.5 + 16 (16.8) | 0.31 | Synergy |
| PA OU1 | 256 (268) | 64 | 4 + 32 (33.6) | 0.19 | Synergy |

^{a}Oxacillin (OXA) susceptibility breakpoints are resistance ≥ 4 μg/mL; susceptible < 4 μg/mL
^{b}PEG-BPEI = PEG₃₅₀-BPEI₆₀₀ = (PEG-350)₁-(BPEI-600)₁
^{c}Piperacillin (PIP) susceptibility breakpoints are resistance ≥ 32 μg/mL; susceptible < 16 μg/mL
^{d}Piperacillin only, no tazobactam added

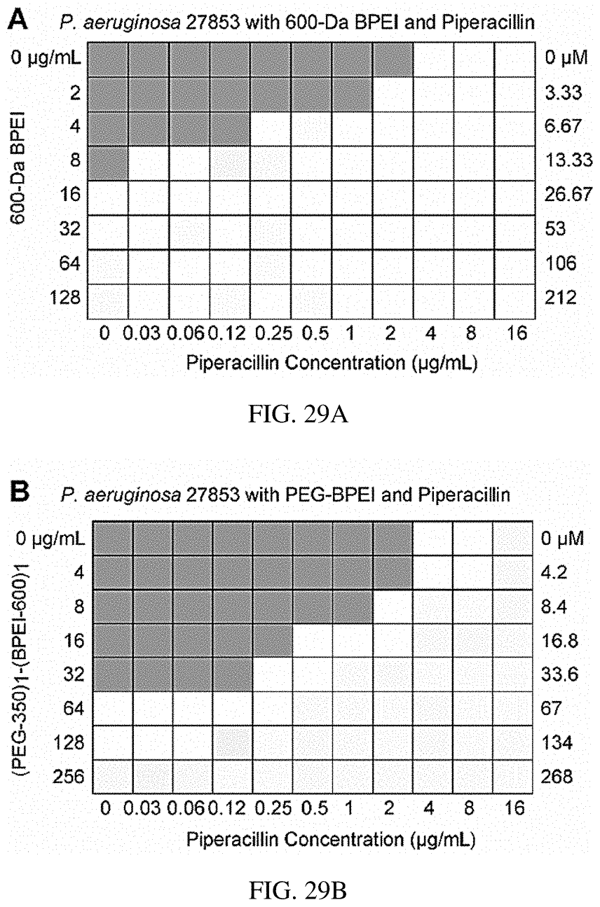

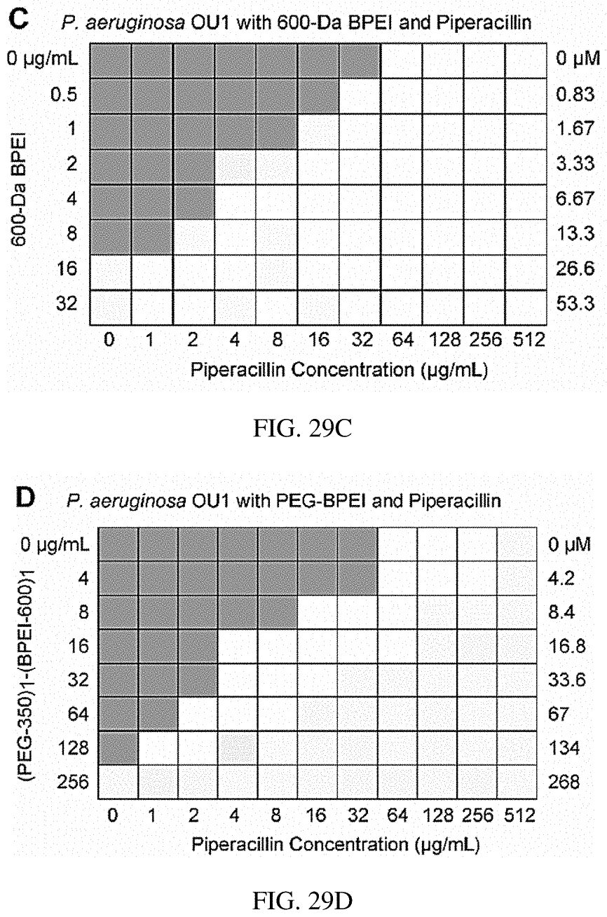

Potentiation of piperacillin against P. aeruginosa is also affected when PEG₃₅₀ is attached to BPEI₆₀₀. The strain P. aeruginosa 27853 is piperacillin susceptible (MIC≤16 μg/mL), and the MPC₄-PIP is 6.67 μM for BPEI₆₀₀ and 16.8 μM for PEG₃₅₀-BPEI₆₀₀ (Table 9). Against the P. aeruginosa clinical isolate OU1, which is multi-drug resistant (Lam, A. K.; Panlilio, H.; Pusavat, J.; Wouters, C. L.; Moen, E. L.; Rice, C. V., Overcoming Multidrug Resistance and Biofilms of Pseudomonas aeruginosa with a Single Dual-Function Potentiator of beta-Lactams. Acs Infect Dis 2020), the MPC₄-PIP of BPEI₆₀₀ is 1.67 μM while 3.33 μM lowers the piperacillin MIC to 8 μg/mL which indicates antibiotic susceptibility (FIGS. 29A-29D). However, PEG₃₅₀-BPEI₆₀₀ is less effective, as the MPC₄-PIP is 8.4 μM and it takes 16.8 μM of PEG₃₅₀-BPEI₆₀₀ to lower the piperacillin MIC to levels considered antibiotic susceptible (FIGS. 29A-29D). The MOA for β-lactam potentiation involves BPEI₆₀₀ binding to the anionic LPS in the outer membrane of P. aeruginosa. The phosphate and carboxylate groups of LPS are located on the lipid A and core oligosaccharides, approximately 1-2 nm away from the acyl chains. These anionic sites allow for the chelation of metals that stabilize the LPS layer and provides targets for BPEI₆₀₀ binding. Cationic polymyxin-B and colistin also bind to these sites, but their hydrophobic alkyl tails penetrate the LPS acyl chain region to disrupt membrane integrity and cause widespread catastrophic disruption. The MIC for polymyxins are low, 1-3 μg/mL. In contrast, BPEI₆₀₀ has weaker antimicrobial action (MIC>26 μM, 16 μg/mL) because, without hydrophobic regions, it does not disrupt the membrane. Instead, BPEI₆₀₀ increases the ability of β-lactams to traverse the O-antigen and core oligosaccharides of LPS and reach porin transporters. It is likely that PEG₃₅₀-BPEI₆₀₀ shares this MOA. The higher MIC and slightly weaker potentiation property suggest that interactions between LPS and BPEI are reduced by PEGylation.

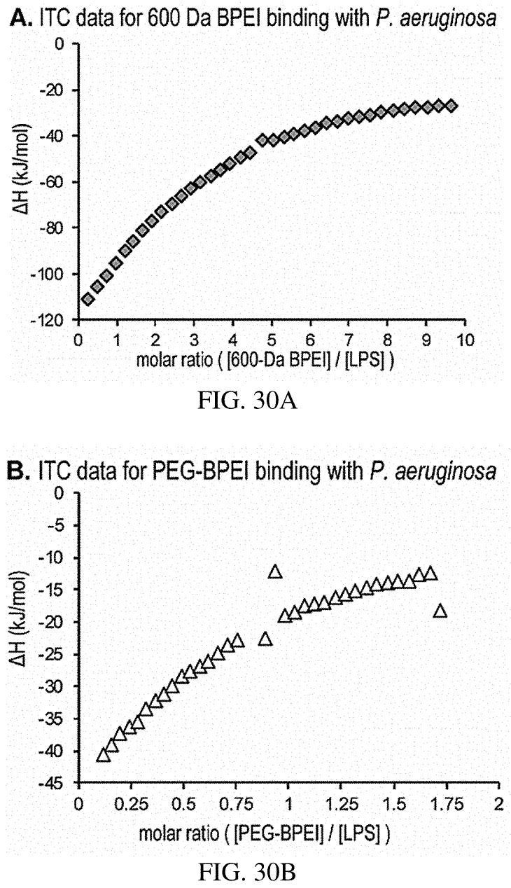

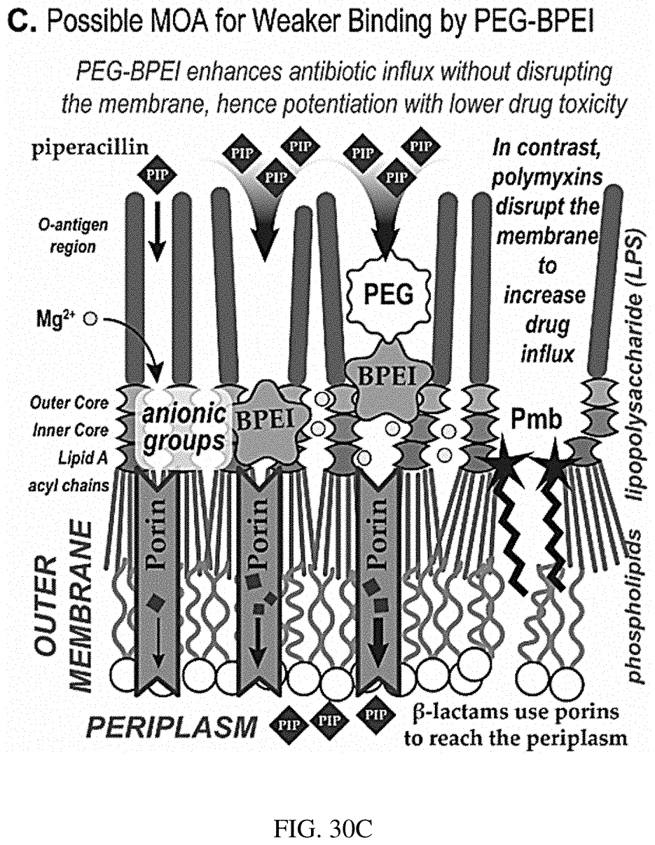

Isothermal Titration calorimetry (ITC) directly measures the enthalpy of molecular binding interactions. We used ITC to confirm interactions between BPEI₆₀₀ and LPS. Without wishing to be bound by theory, we posit a LPS-binding MOA for PEG-BPEIs. The isotherm obtained from a titration of PEG₃₅₀-BPEI₆₀₀ with P. aeruginosa LPS (Sigma #L8643) is shown in FIGS. 30A-30C. The negative ΔH values indicate exothermic binding. However, when compared to the isotherm for BPEI₆₀₀ (FIG. 30A), PEG-BPEI has a less exothermic interaction with P. aeruginosa LPS (FIG. 30B). Likewise, the molar ratio of PEG-BPEI to LPS is approximately is lower than that observed with BPEI₆₀₀. These data demonstrate that PEG₃₅₀-BPEI₆₀₀ does bind with LPS but that PEGylation reduces binding energetics and the ability of a single BPEI₆₀₀ molecule to bind with multiple LPS molecules. This is not surprising as the PEG group would form a large steric barrier to shield some cationic amines from their anionic targets while allowing other amines to bind with LPS. This weakening of LPS binding may explain why PEGylation of BPEI₆₀₀ reduces antibiotic potentiation (FIG. 30C). More PEG₃₅₀-BPEI₆₀₀, (17 μM), than BPEI₆₀₀ (3.3 μM), is needed to potentiate piperacillin against MDR-PA (FIGS. 29A-29D). This weakness is mitigated by considering that PEG₃₅₀-BPEI₆₀₀ has lower in vivo toxicity (MTD=75 mg/kg) than BPEI₆₀₀ (MTD=25 mg/kg); and as discussed below, does not cause β-lactam ring hydrolysis but does possess superior anti-biofilm properties. Without wishing to be bound by theory, it is possible that the PEG group inhibits the active moiety, BPEI₆₀₀, from reaching the phosphates of lipid A at the acyl chain interface. This scenario may also explain why PEGylation increases drug safety, perhaps by preventing PEG-BPEI from disruption eukaryotic membranes.

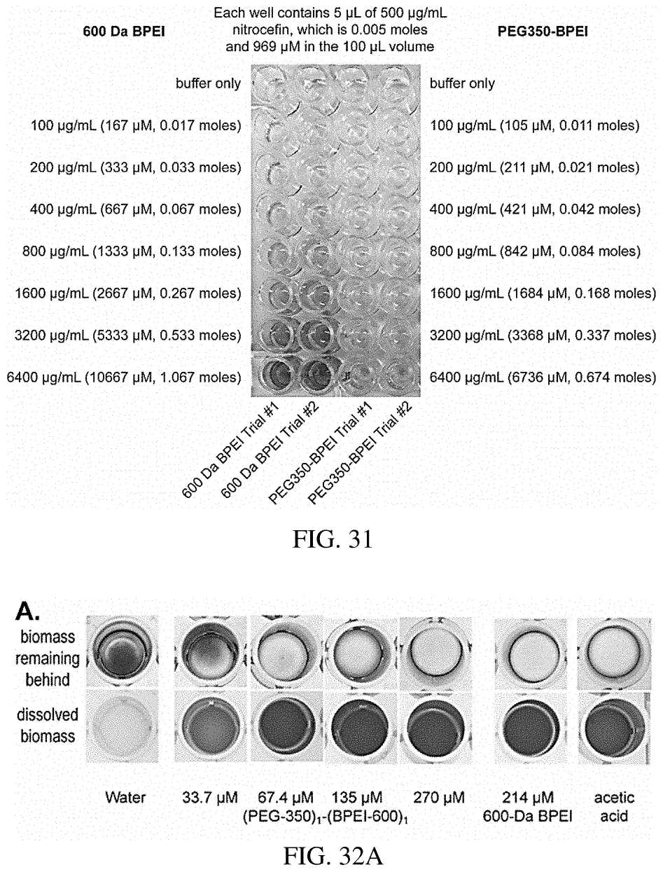

The ability of PEGylation to increase safety and lower the acute toxicity are strong benefits that outweigh any reduction in potentiation efficacy. Because an important use of PEG₃₅₀-BPEI₆₀₀ would be as a topical application to acute and chronic wounds containing MRSA, MRSE, and/or MDR-PA bacteria, higher drug concentrations can be directly applied to the wound. As noted above, PEG-BPEI exposure to subcutaneous tissue does not cause adverse toxicity. Furthermore, the benefits of PEG₃₅₀-BPEI₆₀₀ extend beyond disabling β-lactam resistance. Primary amino groups could disrupt the β-lactam ring of the antibiotics. A colorimetric assay of β-lactam hydrolysis was performed with nitrocefin, a chromogenic cephalosporin whose β-lactam ring which is susceptible to β-lactamase mediated hydrolysis. Once hydrolyzed, the degraded nitrocefin compound rapidly changes color from yellow to red. As shown in FIG. 31, the unmodified BPEI₆₀₀ causes slight hydrolysis at a molar ratio of 0.017:0.005 (3.4:1) whereas PEG₃₅₀-BPEI₆₀₀ has a similar effect at a molar ratio of 0.168:0.005 (33.6:1). Thus, PEGylation of BPEI leads to a 100× reduction in hydrolytic activity of the constrained β-lactam ring of nitrocefin. Bacterial biofilms play a major role the ability of AMR pathogens to withstand antibiotic therapy. They deploy a protective layer of extracellular polymeric substances (EPS) composed of polysaccharides, extracellular DNA, and proteins. These biomacromolecules are crosslinked and encase bacteria. The resulting matrix hinders the diffusion and accessibility of antibiotics and host immune agents. Treating wound biofilms often involves antibiotic therapy plus mechanical debridement and irrigation with saline that may contain detergents. The presence of MRSA, MRSE, and/or MDR-PA renders many standard-of-care antibiotics useless. Bacterial cells that remain after cleansing survive antibiotic therapy quickly populate the wound bed and regenerate the biofilm matrix. An advantage of BPEI₆₀₀ is that PEGylated BPEI₆₀₀ has a superior anti-biofilm properties against staphylococci and P. aeruginosa when compared to non-PEGylated BPEI₆₀₀.

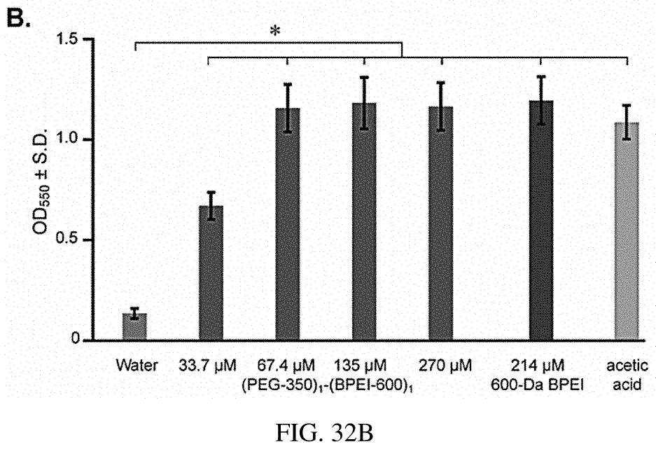

Data from a crystal violet biofilm assays are shown in FIGS. 32A-32B. MRSE 35984 produces strong and consistent biofilms. Biofilms were stained with crystal violet and treated with PEG₃₅₀-BPEI₆₀₀, BPEI₆₀₀, water, and acetic acid (FIG. 32A). The supernatant was carefully transferred into a new plate for the OD₅₅₀ measurement, which corresponds to the amount of dissolved biofilm (FIG. 32B). The biofilm is dissolved with 214 μM (128 μg/mL) of BPEI₆₀₀. Adding PEG₃₅₀ to BPEI₆₀₀ further improves its anti-biofilm properties. The MRSE 35984 biofilm is completely dispersed by 67.4 μM of PEG₃₅₀-BPEI₆₀₀, a concentration that is 3.6 times lower than the 214 μM of BPEI₆₀₀ required to give the same results. This highlights the biofilm disrupting potential of PEG₃₅₀-BPEI₆₀₀ and that PEGylation improves disruption. The biofilm EPS of staphylococci contains a large component of poly-N-acetyl glucosamine (PNAG) and anionic extracellular teichoic acid and cDNA. These components facilitate and stabilize biofilm formation. The primary amines of PEG₃₅₀-BPEI₆₀₀ bind with anionic EPS moieties to disrupt biofilm integrity and stability. The hydrophilic nature of PEG₃₅₀-BPEI₆₀₀ increases the ability of antibiotics to penetrate the biofilm matrix while simultaneously causing the biofilm to disperse. The staphylococci cells become vulnerable to β-lactam antibiotics when additional PEG₃₅₀-BPEI₆₀₀ molecules bind to the planktonic cells and disable PBP2a/4 resistance mechanisms.

Importantly, biofilms can be eradicated without dissolving the EPS. For MRSE, one can overcome oxacillin resistance in planktonic cells where the MIC drops from 32 to 4 μg/mL with 6.67 μM BPEI₆₀₀ and 33.37 μM of PEG₃₅₀-BPEI₆₀₀. Eradication of MRSE biofilms requires a higher amount of oxacillin, MBEC=512 μg/mL, because of barriers imposed by the biofilm EPS. However, BPEI₆₀₀ can weaken the EPS to increase oxacillin activity without dissolving the EPS. The oxacillin MBEC drops to 16 μg/mL in the presence of 13 μM of BPEI₆₀₀. However, this 13 μM of BPEI₆₀₀ does not dissolve the biofilm according to the crystal violet assay. Rather, 214 μM of BPEI₆₀₀ are required to disperse the biofilm EPS into solution. In the MBEC assay using the Calgary biofilm device, biofilms are grown on polystyrene prongs on the lid of a 96-well plate. After biofilms are established on the prongs, they are transferred to a 96-well plate for treatment before transfer to a third plate of media only, where sonication is used to dislodge the biofilms from the prongs. Biofilms that remain attached to the prongs during the treatment phase are weakened by the treatment solution. In this case, 13 μM of BPEI₆₀₀ was able to weaken the MRSE biofilm, allowing 16 μg/mL of oxacillin to kill the cells in the biofilm EPS that remained attached to the prong.

### Example 4

Effects of BPEI on Planktonic and Biofilm MRSAs

Results in this example demonstrate the ability of BPEI₆₀₀ to eradicate MRSA biofilms. Other work shown herein shows that this low-molecular-weight BPEI exhibits low in vitro cytotoxicity on human cells, and strong potentiation with β-lactam antibiotics against planktonic MRSA cells. Strong synergy was also found against MRSE and its biofilms. On this basis it was hypothesized that BPEI would potentiate ampicillin against MRSA biofilms via similar biochemical mechanisms. As described below, BPEI demonstrates strong efficacy against two biofilm-forming MRSA clinical isolates (MRSA OU6 and OU11) that are strongly resistant to antibiotics (see Table 10).

TABLE 10
MRSA clinical isolates OU6 and OU11 demonstrate strong resistance against antibiotics.
MRSA Clinical Isolate Data Collected at OUHSC Clinical Microbiology Laboratory

| | | Methicillin | Clindamycn | | Daptomycin | | Erythromycin | |
|---|---|---|---|---|---|---|---|---|
| Isolate | Species | Resistant | MIC | Interp | MIC | Interp | MIC | Interp |
| 6 | S. aureus | Y | R | >4 | S | ≤0.5 | R | >4 |
| 11 | S. aureus | Y | R | >4 | S | ≤0.5 | R | >4 |

| | | Methicillin | Gentamicin | | Linezolid | | Oxacillin | |
|---|---|---|---|---|---|---|---|---|
| Isolate | Species | Resistant | MIC | Interp | MIC | Interp | MIC | Interp |
| 6 | S. aureus | Y | S | ≤4 | S | 2 | R | >2 |
| 11 | S. aureus | Y | S | ≤4 | S | 2 | R | >2 |

| | | Methicillin | Tetracycline | | Trimeth/Sulfa | | Vancomycin | |
|---|---|---|---|---|---|---|---|---|
| Isolate | Species | Resistant | MIC | Interp | MIC | Interp | MIC | Interp |
| 6 | S. aureus | Y | S | ≤4 | S | ≤0.5/9.5 | S | 2 |
| 11 | S. aureus | Y | S | ≤4 | S | ≤0.5/9.5 | S | 1 |

Unless otherwise indicated, identification and susceptibility performed by the Beckman Coulter MicroScan Walkaway 96plus using the PC33 gram positive panel
*Presumed resistant/D test (inducible clindamycin resistance) positive
\hat{ }Species identification and oxacillin/methicillin Susceptibility/Resistance determined by Verigene Gram Positive Blood Culture assay (probes for Genus/species and mecA)

Experimental Procedure

Materials

In this experiment, the Staphylococcus aureus (MRSA 43300) was purchased from the American Type Culture Collection. Two MRSA clinical isolates (MRSA OU6 & OU11) from patient swabs were kindly provided by Dr. McCloskey from the University of Health Sciences Center with an institutional review board (IRB) approval. Chemicals (DMSO, growth media, and electron microscopy fixatives) were purchased from Sigma-Aldrich. Antibiotics (ampicillin and polymyxin B) were purchased from Gold Biotechnology. BPEI₆₀₀ was purchased from Polysciences. MBEC™ Biofilm Inoculators were purchased from Innovotech. Isopore polycarbonate membrane filters (0.1 μm pore size, hydrophilic, 13 mm diameter) were purchased from MilliporeSigma.

MBEC Assay

Bacterial culture was inoculated in an MBEC pronged-inoculator and incubated for 24 hr to allow biofilm formation. Then, the preformed biofilm prong lid was washed and treated in a separate challenge plate which was prepared as a checkerboard assay: serial dilutions of BPEI₆₀₀ and antibiotic solutions were added to a 96-well base plate with a total volume of 200 μL cation-adjusted Muller Hinton broth (MHB) per well. The change in optical density at 600 nm (Δ OD₆₀₀) was measured. Minimum inhibitory concentration (MIC) of each drug is determined as the lowest concentration that inhibited cell growth (ΔOD₆₀₀<0.05). Fractional inhibitory concentration index (FICI) was calculated as:

$$FICI = \frac{{MIC}_{A B}}{{MIC}_{A}} + \frac{{MIC}_{B A}}{{MIC}_{B}} .$$

Synergistic effects are determined using EUCAST guidelines: synergy (FICI≤0.5), additivity (0.5<FICI<1), and indifference (FICI>1). The treated pronged-inoculator was then washed and transferred to a recovery plate with 200 μL MHB/well to sonicate and recover any remaining biofilm bacteria. The recovery plate was then incubated overnight before measuring ΔOD₆₀₀ to determine MBECs and FICIs of the drugs tested on the biofilms.

Biofilm Disrupting Assay

This experiment was conducted in parallel with polymyxin B (PmB, a cationic polypeptide antibiotic) and BPEI₆₀₀. In short, an overnight MRSA OU 6 culture was inoculated in a tissue-culture treated 96-well plate (100 μL of tryptic soy broth or TSB/well) with an inoculation size of 1 μL/well (˜5×10⁵ CFU/mL). The plate was incubated at 35° C. for 24 hr to allow the bacteria to form biofilm. It was then washed with water to remove planktonic bacteria and stained with 100 μL of crystal violet solution (0.1%) per well for 15 min. The stained plate was washed excessively with water 5 times to remove any unbound stain and air-dried overnight. Varying concentrations of PmB (64 and 128 μg/mL) and BPEI₆₀₀ (64 and 128 μg/mL) were added to the stained-biofilm plate with a total volume of 100 μL/well. A negative control (water only) and positive control (30% acetic acid) were also conducted at the same time of treatment. After 20 hr, without touching the biofilm layer in the bottom of the plate, solubilized solution in each treated well was carefully transferred to a new 96-well plate for an OD₅₅₀ measurement, which represents the corresponding amount of biofilm disrupted by each treatment.

Scanning Electron Microscopy (SEM)

MRSA OU6 were inoculated from 0.5% of an overnight culture on glass coverslips and grown at 35° C. After 24 hr. the biofilm-formed on glass coverslips were carefully removed and washed in water for 10 s. Then each sample was submerged in different treated solution (untreated control, 128 μg/mL BPEI-treated, and bleach-positive control) for another 24 hr. Next, they were removed, washed in water for 10 s, and submerged in primary fixative (5% glutaraldehyde in 0.1 M cacodylate buffer) and incubated at 4±2° C. for 2 days. The glass coverslips were removed from the fixing solution and air-dried for 72 hr. They were mounted on aluminum stubs with carbon tape and sputter-coated with AuPd. A Zeiss NEON SEM was used to image the samples at 5 kV accelerating voltage.

SEM of Biofilms on Polycarbonate Membrane Filters

Pre-sterilized polycarbonate (PC) membranes were gently adhered to a tryptic soy agar plate using sterilized forceps. A volume of 2 μL of the stock MRSA OU6 solution (˜5×10⁵ CFU/mL) was pipetted on top of each PC membrane and incubated at 35° C. for 7-8 hr, when the MRSA biofilm colony on the PC membranes became visible to the naked eye. The PC membranes with preformed biofilm was then carefully removed off the agar, transferred into a treatment solution of 256 μg/mL BPEI₆₀₀, and incubated for another 20 hr. Untreated and treated PC samples were removed and washed in water for 10 s. They were submerged in primary fixative (5% glutaraldehyde in 0.1 M cacodylate buffer) and incubated at 4±2° C. for 2 days. The PC samples were air-dried slowly for 3 more days. They were mounted on aluminum stubs with double-side carbon tape, sputter-coated with AuPd, and imaged at 5 kV accelerating voltage by a Zeiss Neon SEM.

Results

MBEC assays were utilized on MRSA OU6 and MRSA OU11 and a lab strain MRSA ATCC 43300. The MRSA bacteria were used to inoculate a 96-well inoculation plate, where MRSA biofilms were grown on prongs protruding from the plate lid, known as the MBEC inoculator lid and based on the Calgary biofilm device. The inoculator lid was washed to remove unattached MRSA cells and transferred into a separate 96-well base for treatment with BPEI₆₀₀ and ampicillin combinations arranged in a checkerboard assay pattern, the so-called the challenge plate. The final step was moving the treated inoculation lid to a third plate (the recovery plate) containing growth-media only and using sonication to dislodge the biofilm and recover cells remaining in the biofilm. In this manner, the synergy of BPEI and ampicillin against MRSA biofilms was evaluated. Standard CLSI (Clinical & Laboratory Standards Institute) guidelines describe a standard MIC assay using 96-well plates inoculated with a standard cell density, usually ˜10⁶ CFU/mL. However, the MIC data reported here is non-standard because, rather than inoculation via micropipette transfer from an overnight culture, inoculation of the challenge plate occurs from the biofilm-coated inoculation lid where treatment challenge disrupts the protective biofilm EPS matrix. MRSA cells are dislodged and dispersed into the challenge plate media. These cells in the challenge plate media are susceptible to killing by the BPEI₆₀₀, ampicillin, or their combinations, and a minimum inhibitory concentration can be determined. This value is referred to as MIC_{CP} to differentiate it from MIC measurements made with standard methods. The MBEC is determined from cell growth in the recovery plate and reflects the ability of BPEI₆₀₀, ampicillin, or its combinations to kill the biofilm remaining attached to the prongs of the inoculation lid. The MIC_{CP} and MBEC data are shown for comparison in Table 11.

As shown in Table 11, MRSA 43300's BPEI₆₀₀ MBEC (>256 μg/mL) is much larger than its MIC_{CP} (64 μg/mL). Similarly, the ampicillin MBEC (>256 μg/mL) is higher than the corresponding MIC_{CP} (128 μg/mL). The MBECs for BPEI₆₀₀ and ampicillin against the two clinical isolates, MRSA OU6 and OU11, are greater than the highest amount tested, 256 μg/mL. Although the MBECs exceeded the tested concentrations, strong synergy (FICI<0.5) was found between BPEI and ampicillin against the biofilms of MRSA 43300, OU11, and OU6 with an FICI of 0.13, 0.25, and 0.19, respectively. For example, when combined with 64 μg/mL of BPEI, the ampicillin MBECs for MRSA 43300, OU6, and OU11 were reduced to 2, 64, and 32 μg/mL, respectively. For these strains, the MIC_{CP} is higher than previously reported values for planktonic MRSA cells evaluated with CLSI methods, which showed that BPEI₆₀₀ lowers the MIC for the planktonic cells and renders them susceptible to oxacillin. As described above, the disparity arises from different methods of inoculation and the cell density in the challenge plate media is unknown and likely varies between wells. Nevertheless, the MIC_{CP} can be used to show that BPEI and ampicillin combinations can be used to kill antibiotic-resistant cells dislodged from the inoculation lid.

TABLE 11
Synergistic effects between BPEI₆₀₀
and ampicillin against MRSA biofilms

| | | | Ampicillin | | | | |
|---|---|---|---|---|---|---|---|
| | | | (μg/mL) | | | | |
| | BPEI | | | | MBEC + | | |
| | (μg/mL) | | | | 64 μg/mL | | Syn- |
| Strain | MIC_{CP} | MBEC | MIC_{CP} | MBEC | BPEI | FICI | ergy? |
| MRSA | 64 | >256 | 128 | >256 | 2 | 0.13 | yes |
| 43300 | | | | | | | |
| MRSA | >256 | >256 | 256 | >256 | 64 | 0.25 | yes |
| OU6 | | | | | | | |
| MRSA | >256 | >256 | 128 | >256 | 32 | 0.19 | yes |
| OU11 | | | | | | | |

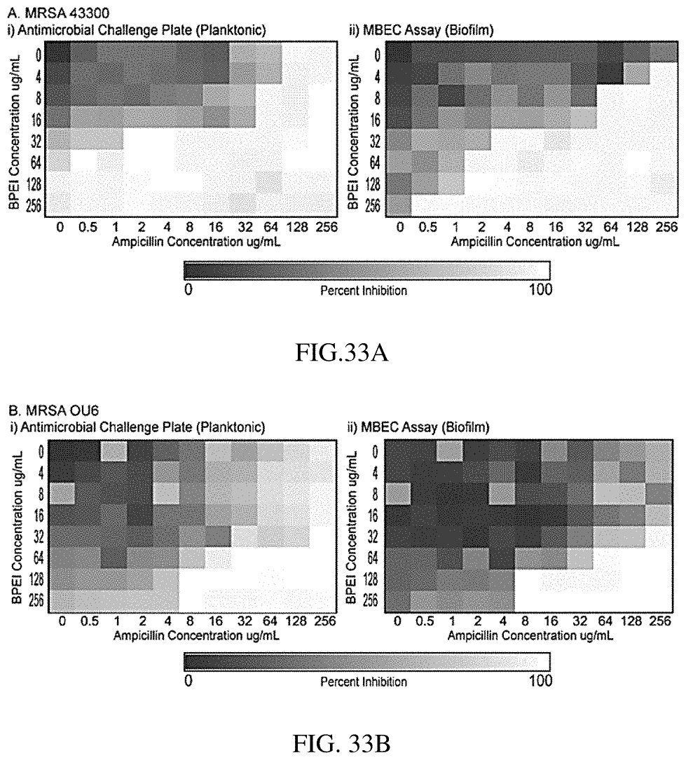

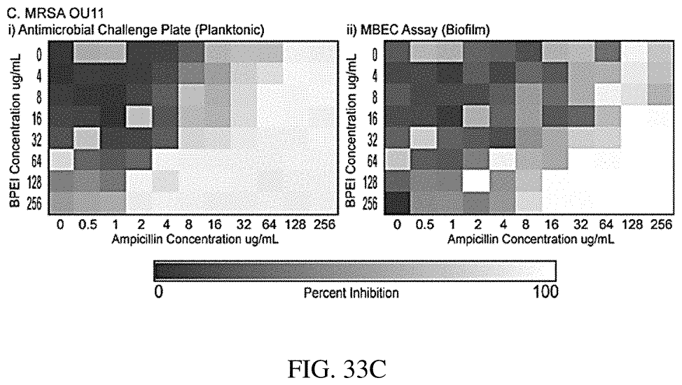

Heatmaps of the average checkerboard results are shown in FIGS. 33A-33C. Data used to determine MIC_{CP} in the challenge plate containing MRSA planktonic data are shown in FIGS. 33A(i), 33B(i), and 33C(i) and the corresponding biofilm data are shown in FIGS. 33A(ii), 33B(ii), and 33C(ii). As expected, the MBECs are larger than the respective MIC_{CP} values. This demonstrates the intrinsic protective nature of biofilms against antimicrobial agents. The stair-case pattern found in the heatmaps indicate that multiple combinations of BPEI₆₀₀ and ampicillin are effective against both planktonic and biofilm forms of MRSA 43300, OU6, and OU11 strains. As BPEI concentration increases, the required MIC_{CP} and MBEC values of ampicillin decrease to achieve high inhibition percentage, highlighting the potentiating ability of BPEI against pathogenic biofilms.

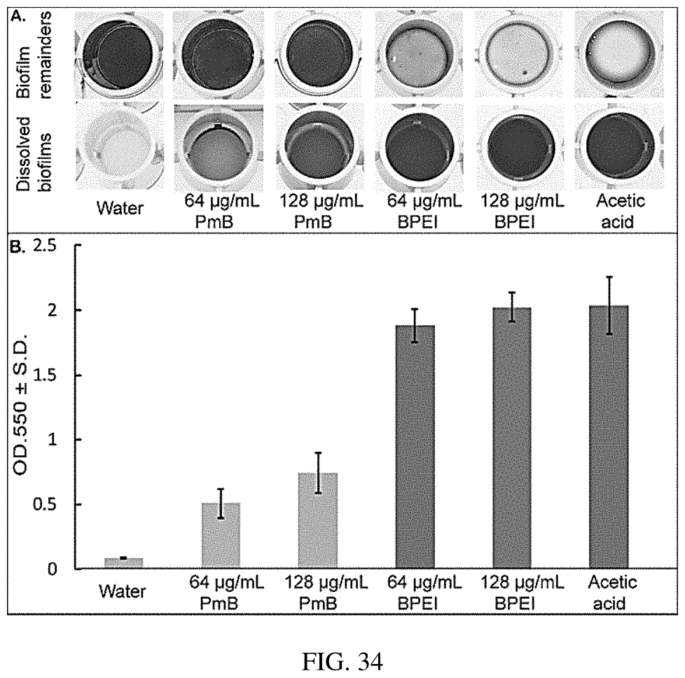

To better elucidate the antibiofilm activity of BPEI, biofilm disruption assays were conducted along with a comparison study using the common cationic antibiotic polymyxin B. Briefly, MRSA OU6 biofilms were grown on the bottom of a 96-well plate for 24 hr. After repeated washing, the biofilms were stained with crystal violet for semiquantitative analysis. The biofilms were then treated to investigate the ability of BPEI or polymyxin-B to disrupt the biofilm. As shown in FIG. 34, the negative control of water only had no impact on disrupting the MRSA biofilms because the biofilm layer remained intact in the bottom (top-down photographic image in FIG. 34(A), upper panel). On the other hand, BPEI₆₀₀ (64 and 128 μg/mL) completely dispersed the MRSA biofilms into its solution in a manner similar to that of the positive control, acetic acid (top-down photographic image in FIG. 34(A), lower panel). However, exposure to polymyxin B, an FDA-approved cationic polypeptide antibiotic, resulted in a slight dissolution in biomass, although 128 μg/mL was more effective than 64 μg/mL. The biofilm-disrupting properties are quantitatively reported as OD₅₅₀ measurements of the amount of biofilm dislodged (FIG. 34(B)). This demonstrates BPEI's ability to eradicate MRSA biofilms by forcing them to detach and disperse its bacterial cells into planktonic culture, where they transition from a persistent quiescent state into a metabolically active realm and thus become vulnerable to antibiotics.

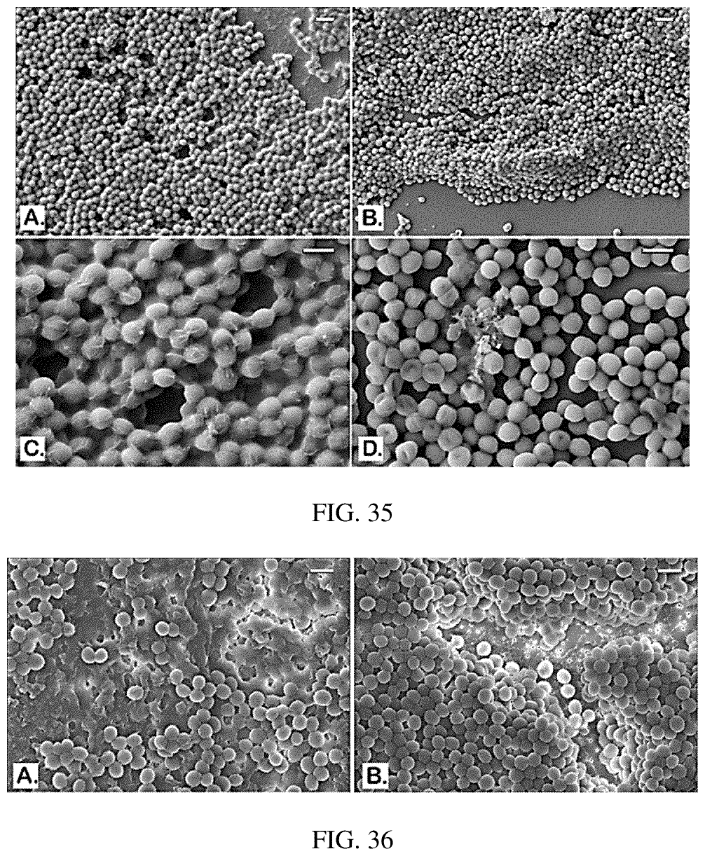

To better characterize the effect of BPEI on MRSA biofilms, morphological analysis was performed using SEM. 24 hr-established MRSA biofilms on glass coverslips were treated with 128 μg/mL of BPEI₆₀₀. An untreated control and the BPEI-treated samples were then fixed and imaged with SEM. As shown in FIG. 35 (A and C), the untreated control MRSA biofilm is enclosed in a thick coat of EPS. Like all biofilm-forming bacteria, the EPS is their self-made protection against harsh environments and antibiotics. With BPEI treatment, the preformed MRSA biofilm lost most of its EPS coat (FIG. 35(B)). At higher magnification (FIG. 35(D)), the lack of EPS in the treated sample rendered visible the inner layers of the bacteria, which were hidden in the untreated control. To mimic a wound environment, MRSA biofilms were grown on polycarbonate (PC) membrane filters (0.1 μm pore size) placed directly on tryptic soy agar. The membrane pores allow for nutrient absorption and we found that these biofilms are more robust than those grown on glass slides. In the untreated control sample (FIG. 36(A)), the EPS is so thick that the SEM scan cannot locate the bottom of the PC membrane filter. In BPEI-treated sample (FIG. 36(B)), many areas are exposed from the absence of EPS, including the bottom surface of the membrane filter whose nano-size pores (tiny white dots through the crack in (FIG. 36(B)) are clearly visible.

The biofilm EPS of S. aureus contains a high fraction of polysaccharide intracellular adhesin (PIA) and anionic species that are prime targets for BPEI₆₀₀ binding, such as eDNA and extracellular teichoic acid (TA). The latter is a key component in the biofilm EPS matrix of S. epidermidis and S. aureus. It enhances bacterial adhesion to biotic and artificial surfaces, which is the first step of biofilm formation. TA has a negative net charge at neutral pH because it contains more negatively-charged phosphates than positively-charged D-alanine residues. Using nuclear magnetic resonance spectroscopy, we found that BPEI₆₀₀ electrostatically binds wall teichoic acid, which indirectly hinders the resistance factor PBP2a/4. Similarly, BPEI most likely binds extracellular TA in the EPS matrix, and also eDNA, to disrupt biofilm structural integrity, as seen in FIGS. 35-36. The exposure of individual bacteria could enhance their contact with various drugs and components of the immune system.

Skin or soft-tissue infections (SSTIs) arise from abrasions, non-surgical wounds, burns, or chronic health problems. For chronic wound infections associated with MRSA and its biofilm, treatment options are scarce. Patients afflicted with these chronic wounds suffer from physical pain and disabilities in addition to psychological and emotional stresses and poor quality of life. Current in-patient treatments include cleansing, debridement, maintaining a moist tissue environment, and when possible, eliminating the underlying pathology or factors that contributed to poor wound healing. In advanced cases, amputation may become necessary. Death, especially in elderly patients, may result from sepsis that can be associated with chronic wounds. Antibiotics can be used effectively against susceptible infections. For drug-resistant infections, the best-practices for effective in-patient intervention are strict sanitary guidelines and antibiotics, such as intravenous vancomycin plus piperacillin/tazobactam or IV-treatment with new antibiotics of last resort. Nevertheless, biofilms and antimicrobial resistance create substantial technological barriers to treating chronic wound infections. This presents a significant and critical need for way to counteract biofilms and antimicrobial resistance. BPEI₆₀₀ is a dual-function potentiator because it disrupts biofilms that are otherwise impenetrable to antibiotics, and also counteracts β-lactam resistance mechanisms in MRSA. However, success requires that BPEI₆₀₀ have low toxicity. In dermal applications, low-molecular-weight BPEI was shown to have high biocompatibility and low genotoxic potential. We also confirmed the non-cytotoxicity of BPEI₆₀₀ toward human kidney, colon, and HeLa cells with the IC₅₀ of 1090 and 690 μg/mL on human HeLa cells and HEK293, respectively. Additionally, lactate dehydrogenase (LDH) assays showed that BPEI₆₀₀ gave the lowest nephrotoxicity of 3.5% at 63 μg/mL (even lower than Polymyxin E/Colistin which was >20% nephrotoxicity at the same concentration tested). With bacterial evolution outpacing the discovery of antimicrobial agents, it is imperative to seek alternative treatments options, such as coupling existing drugs with potentiators. With a dual-function mechanism that eliminates antibiotic efficacy barriers in both planktonic and biofilm-encased bacteria, BPEI₆₀₀ has promise as a therapeutic agent for improving wound care and combating medical device infections. Potency of first-line antibiotics such as ampicillin can now be restored by the addition of BPEI against drug-resistant MRSA, as seen by their strong synergistic effects. Combinations of BPEI and antibiotics could be administered to diagnosed or suspected staph-biofilm infections, which would improve efficacy and treatment of resistant, biofilm-forming, pathogens.

### Example 5

Effects of BPEI on Drug-Resistant Pseudomonas aeruginosa

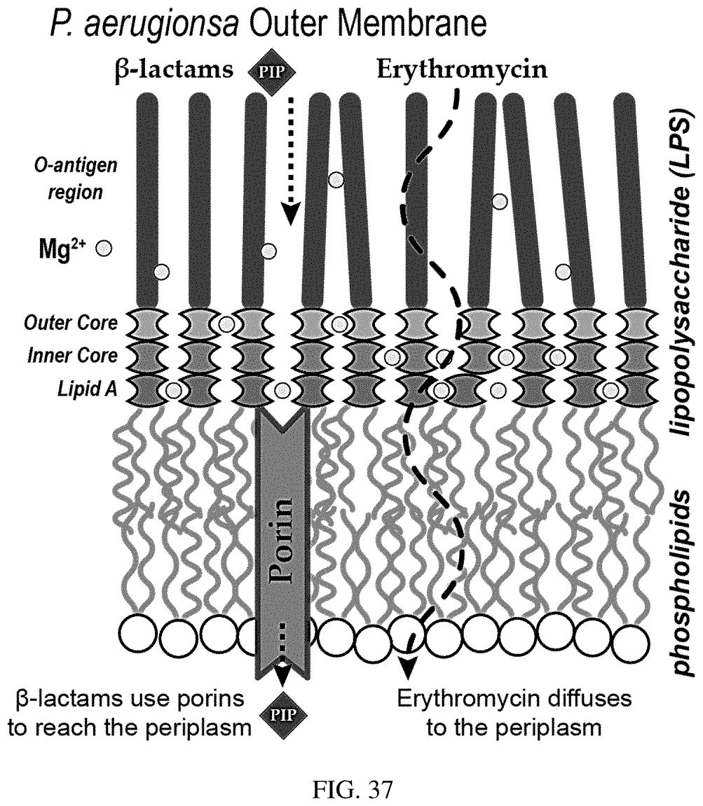

Pseudomonas aeruginosa is a Gram-negative bacterium for which antibiotic therapy is useful, but resistant strains often result in severe chronic infections. It poses a great risk to public health because its outer membrane, composed of lipopolysaccharides (LPS), is a barrier to antibiotic influx (FIG. 37). P. aeruginosa causes severe pneumonia, bloodstream infections, respiratory tract infections (RTIs), urinary tract infections (UTIs), skin infections, and eye infections. Commonly found in burn units, P. aeruginosa is of particular concern in wound healing because it produces biofilms that are impenetrable to antibiotics, leading to chronic infections. Biofilms sequester bacterial pathogens and protect them from antimicrobial attack. They are associated with ear infections, chronic sinus infections, abrasions, wound infections, burns, or chronic health problems. For example, infections of diabetic wounds and foot ulcers often become chronic because they stall in suboptimal inflammatory phase of healing perpetuated by biofilms. aeruginosa infections and their biofilms create serious health issues, and the threat to patient survival increases when the bacterium is multidrug-resistant P. aeruginosa (MDR-PA).

Biofilms and antibiotic resistance create substantial technological hurdles to patient treatment. This presents a significant and critical need for way to counteract them. Existing drugs and regimens are coupled with potentiators that overcome antibiotic resistance or biofilms. However, it is possible to develop a single compound that disables biofilms and combats antibiotic resistance. As a multi-purpose potentiator, BPEI₆₀₀ can disable resistance and dissolve their biofilms. We have used BPEI₆₀₀ to confront the biofilm directly and disrupt the protective exopolymer substances (EPS) network of methicillin-resistant staphylococci while simultaneously counteracting β-lactam resistance mechanisms. This example shows that BPEI₆₀₀ also disables MDR mechanisms, and biofilms, in P. aeruginosa obtained from the American Type Culture Collection (ATCC) and antibiotic resistant clinical isolates.

Methods

Materials

Pseudomonas aeruginosa bacteria were purchased from the American Type Culture Collection (ATCC BAA-47 and 27853). Additional MDR-PA strains were obtained from clinical isolates from the University of Oklahoma Health Sciences Center using appropriate IRB protocols and procedures. Wild-type P. aeruginosa PAO1 and its efflux pump-deficient mutant, PaΔ3, were kindly provided by Prof. Helen Zgurskaya, University of Oklahoma. Chemicals were purchased from Sigma-Aldrich (DMSO, growth media, and electron microscopy fixatives). Antibiotics were purchased from Gold Biotechnology. BPEI₆₀₀ was purchased from Polysciences, Inc. MBEC™ Biofilm Inoculator with 96-well base plates were purchased from Innovotech, Inc.

Checkerboard Assays and Growth Curves

Checkerboard assays were used to determine the synergistic effect between BPEI₆₀₀ and antibiotics against the P. aeruginosa strains growing in cation-adjusted Mueller-Hinton broth (CAMHB). Bacterial growth curves were obtained using CAMHB media augmented with various amounts of BPEI₆₀₀ and/or piperacillin inoculated with P. aeruginosa BAA-47 cells from an overnight culture (5×10⁵ CFU/mL). Cells were grown at 35° C. with shaking. The OD₆₀₀ (optical density at 600 nm) was monitored and recorded for each sample over 24 hr. Each checkerboard trial was done in triplicate using sterile Greiner CellStar™ flat bottom polystyrene plates, catalog #655180. Each growth curve was done in duplicate.

Inoculation and Biofilm Formation

A sub-culture of P. aeruginosa BAA-47 was grown from the cryogenic stock on an agar plate overnight at 35° C. The MBEC plate was inoculated with 150 μL of CAMHB/well plus 1 μL of a stock culture made from 1 colony/mL of P. aeruginosa BAA-47 in CAMHB (˜5×10⁵ CFU/mL). The MBEC inoculator plate was sealed with Parafilm™ and incubated for 24 hr at 35° C. with 100 rpm shaking to facilitate biofilm formation on the prongs. Following biofilm formation, the lid of the MBEC inoculator was removed and placed in a rinse plate containing 200 μL of sterile PBS for 10 sec.

Antimicrobial Challenge

A challenge plate was made in a new pre-sterilized 96-well plate in a checkerboard-assay pattern to test the synergistic activity of BPEI₆₀₀+antibiotic combinations. Antimicrobial solutions were serial-diluted and added to the 96-well plate, which contained 200 μL of CAMHB per well. After the rinsing step, the preformed biofilm prong lid was immediately transferred into the prepared antimicrobial challenge plate and incubated at 35° C. for 20-24 hr.

Recovery and Quantitative MBEC

After the challenge period, the MBEC inoculator lid was washed and transferred into a recovery plate containing 200 μL of CAMHB per well, sonicated on high (Branson B-220, frequency of 40 kHz) for 30 minutes to dislodge the biofilm and then incubated at 35° C. for 20-24 hr to allow the surviving bacterial cells to grow. After incubation, the OD₆₀₀ of the recovery plate was measured using a Tecan Infinite M20 plate reader to determine the MBEC of the antimicrobial compounds tested. A change in OD₆₀₀ greater than 0.05 indicated growth. Likewise, the OD₆₀₀ for the base of the challenge plate was measured to determine the MICs of the antimicrobial compounds. The fractional inhibitory concentration index (FICI) calculated based on the established equation was used to determine synergy (FICI≤0.5), additivity (0.5<FICI<1), and no synergy (FICI≥1).

Scanning Electron Microscopy

P. aeruginosa BAA-47 cells were inoculated from an overnight culture (5×10⁵ CFU/mL) and grown at 35° C. with shaking. The bacteria were grown in four separate sub-lethal treatments: BPEI₆₀₀ (4 μg/mL), piperacillin (1 μg/mL), combination (4 μg/mL 600-Da BPEI+1 μg/mL piperacillin), and untreated control. Growth was stopped at late-lag phase. Samples were collected by centrifugation and fixed with Karnovsky fixative (2% glutaraldehyde and 2% paraformaldehyde in 0.1M cacodylate buffer) for 30 min. The cells were then fixed with 1% OsO₄ for 30 min in the dark. The cells were washed with water three times. A couple drops of each sample were placed on clean, poly-L-lysine coated coverslips and air-dried for 30 min. The samples were dehydrated by going through a series of ethanol solutions (20%, 35%, 50%, 70%, and 95%), spending 15 min in each solution. Afterward, the samples were dried with hexamethyldisilazane (HMDS). They were then mounted on aluminum stubs with carbon tape and sputter coated with AuPd. A Zeiss NEON SEM was used to image the samples at 5 kV accelerating voltage.

Isothermal Titration calorimetry (ITC)

An isothermal titration calorimeter (MicroCal PEAQ-ITC, Malvern Inc., Malvern, U.K.) was used to assess P. aeruginosa Lipopolysaccharide (LPS) binding with BPEI₆₀₀. Solutions of BPEI (0.64 mg/mL) and L8643 P. aeruginosa LPS (5 mg/mL) were prepared in 50 mM Tris-HCl (pH 7) buffer at 25° C. Titrations were carried out at 25° C. using injections of 2 μL that lasted 4 s and separated by 150 s time intervals. For each experimental setup, controls were performed in which the titrant was injected into pure buffer, buffer was injected into the cell and buffer injected into pure buffer. The experiment was done in duplicate.

H33342 Bisbenzimide and NPN Accumulation Assays

Overnight culture of P. aeruginosa BAA-47 was used to inoculate fresh CAMHB media for another 5 hr at 35° C. with shaking. Bacterial cells were collected by centrifugation at 6000 rpm for 40 min and resuspended in PBS. The OD₆₀₀ of the cell suspension was adjusted to ˜1.0 and kept at room temperature during the experiment. Aliquots (180 μL/well) of the cell suspension were transferred to a 96-well flat-bottom black plate in the format of column 1, PBS blank; column 2, untreated control cells BAA-47; column 3, cells BAA-47+BPEI (sub-lethal concentration). Five technical replicates of each group were conducted. Fluorescent probes Hoechst 33342 bisbenzimide (H33342) or 1-N-phenylnaphthylamine (NPN) was added (20 μL) to each well with a final concentration of 5 μM. Fluorescence was read immediately after the addition of H33342 or NPN by a Tecan Infinite M20 plate reader with the excitation and emission filters of 355 and 460 nm for H33342 or 350 and 420 nm for NPN, respectively. Fluorescence data were normalized to the emission before cells were added in the PBS control, and they were plotted against time to show the cellular uptake of H33342 or NPN over 10 min. Control experiments of dye+BPEI₆₀₀ were unchanged from fluorescence emission values obtained with dye only.

Results

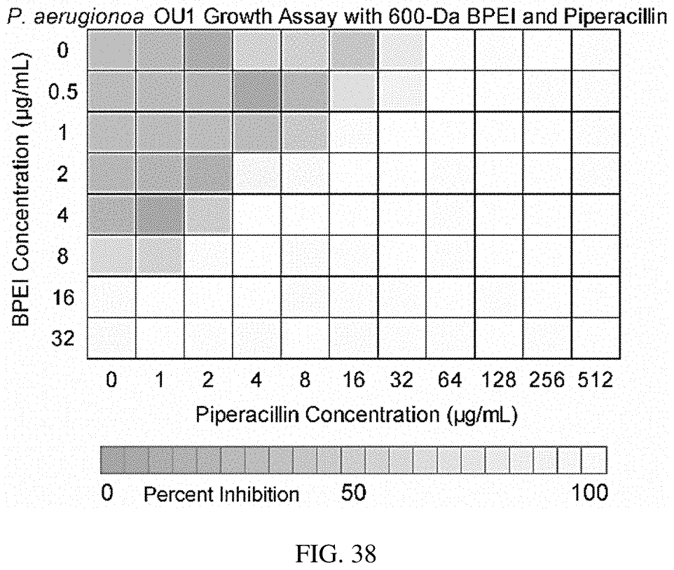

While examining BPEI potentiation of β-lactams against multidrug-resistant P. aeruginosa, checkerboard assays demonstrated synergistic effects between BPEI₆₀₀ and β-lactam antibiotics against two laboratory strains of P. aeruginosa, ATCC 27853 and ATCC BAA-47, and several MDR clinical isolates from patients at the University of Oklahoma College of Medicine. The MICs of BPEI₆₀₀ and piperacillin against these strains were determined and used to calculate the FICIs. An FICI lower than 0.5 indicates synergy while an FICI between 0.5 and 1 represents additivity. The BPEI₆₀₀ MICs against P. aeruginosa ATCC 27853, ATCC BAA-47, and 5 clinical isolates varied from 8 to 64 μg mL⁻¹ (Table 12 and FIG. 38). For the β-lactam antibiotic piperacillin, resistance in P. aeruginosa is defined by USCAST as a minimum inhibitory concentration (MIC)≥8 μg/mL. As shown in Table 12, the ATCC strains were susceptible to piperacillin yet the clinical isolates exhibited strong piperacillin resistance. Using checkerboard assays (FIG. 38), the presence of BPEI₆₀₀ lowered the MIC of piperacillin against MDR-PA isolate OU1 and the other tested strains. The clinical isolates are multidrug-resistant, and all were rendered susceptible to piperacillin with the exception of OU15. Fortunately, we were able to restore susceptibility of OU15 to cefepime, whose 32 μg/ml MIC (resistant) is lowered to 0.5 μg/mL with 16 μg/mL of BPEI₆₀₀. Cefepime resistance in OU19 and OU22 (MIC=64 and 128 μg/mL, respectively) is also eliminated with 16 μg/mL of BPEI₆₀₀ (MIC lowered to 8 μg/mL for both strains).

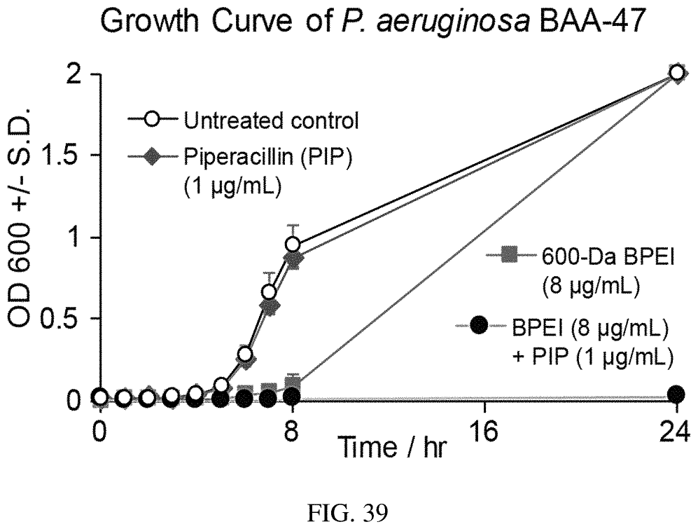

The data in Table 12 were collected without tazobactam, a β-lactamase inhibitor, suggesting that enzymatic activity cannot maintain this form of β-lactam resistance. Perhaps the intracellular piperacillin concentration is sufficient to overcome losses from β-lactamase hydrolysis. Sublethal concentrations of piperacillin become bacteriostatic when combined with sub-lethal concentrations of BPEI₆₀₀ (FIG. 39). Within 24 hours, the untreated control group grew to an OD₆₀₀ of 2, as so did the individual treatment of either BPEI₆₀₀ or piperacillin alone, indicating that these concentrations are insufficient to kill the bacteria. Only the combination BPEI₆₀₀+piperacillin treatment could effectively stop its growth, highlighting the restorative value of BPEI₆₀₀ on β-lactam antibiotic efficacy.

TABLE 12
MIC and FICI values for P. aeruginosa treated
with BPEI₆₀₀, piperacillin, and combinations.

| | MIC [μg/mL] | | | | | | |
|---|---|---|---|---|---|---|---|
| | | PIP/ | | PIP^{a,c} + | | | |
| Strain | BPEI₆₀₀ | TAZO^{a,b} | PIP^{a,c} | BPEI₆₀₀ | | FICI | Outcome |
| PA 27853 | 16 | — | 4 | 0.25 + 4 | μg/mL | 0.31 | Synergy |
| PA BAA-47 | 32 | — | 4 | 1 + 8 | μg/mL | 0.50 | Synergy |
| PA OU1 | 16 | 64 | 64 | 4 + 2 | μg/mL | 0.31 | Synergy |
| PA OU12 | 8 | 128 | 128 | 8 + 4 | μg/mL | 0.31 | Synergy |
| PA OU15 | 32 | n.d. | 128 | 32 + 8 | μg/mL | 0.5 | Synergy |
| PA OU19 | 64 | n.d. | >256 | 1 + 16 | μg/mL | 0.31 | Synergy |
| PA OU22 | 64 | n.d. | >256 | 4 + 16 | μg/mL | 0.37 | Synergy |

^{a}Piperacillin (PIP) susceptibility breakpoints are resistance ≥ 8 μg/mL; susceptible < 8 μg/mL
^{b}Determined by the OUHSC Clinical Microbiology laboratory; TAZO = tazobactam
^{c}Determined in this work; piperacillin only, no tazobactam added
n.d. = not determined

As described below, BPEI₆₀₀ does not inhibit efflux pumps. However, there are concentration dependent effects of BPEI₆₀₀, which has antibiotic properties at high concentration. At lower concentrations used for β-lactam potentiation, the mechanism of action likely involves creating new avenues of access through the LPS layer to increase intracellular antibiotic concentrations and overcome β-lactamase enzymes and efflux pumps. At slightly higher concentrations needed to potentiate erythromycin, BPEI₆₀₀ causes slight perturbations to the outer membrane. However, previous data collected with fluorescence microscopy show that sub-MIC concentrations of BPEI₆₀₀ do not accumulate within E. coli cells.

The ability of improve β-lactam efficacy at low concentration occurs because the cross-linked network of LPS presents a barrier to the free diffusion of antibiotics. The outer membrane of P. aeruginosa contains numerous beta barrel proteins amongst the alkyl chains of the phospholipid and LPS leaflets. These porins allow for the influx of β-lactam antibiotics between the extracellular milieu and the periplasmic space. However, the inner-core, outer-core, and O-antigen regions of LPS slow the uptake of β-lactams. Ca²⁺ and Mg²⁺ ions stabilize these anionic regions and we posit that BPEI₆₀₀ also binds to these sites causing localized reduction in the diffusion barrier. This was evaluated by determining if BPEI₆₀₀ binds to LPS and by performing permeation assays that monitor the intracellular concentration of probe molecules.

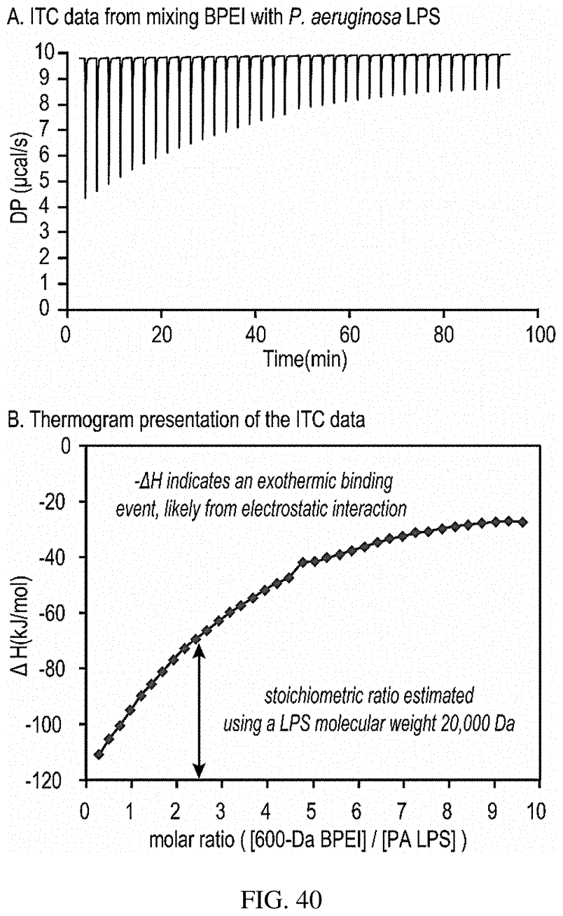

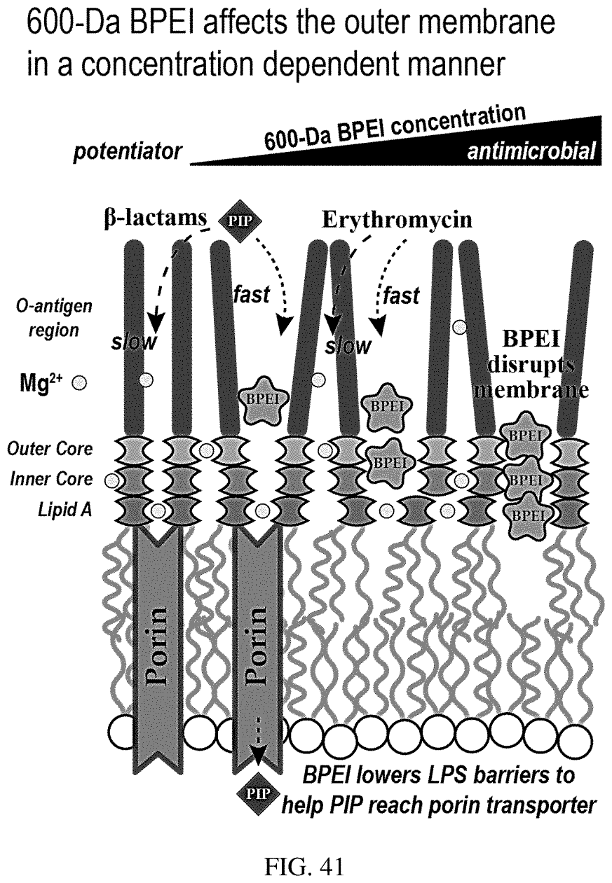

Isothermal Titration calorimetry (ITC) directly measures the enthalpy of molecular binding interactions. Here, it was used to confirm interactions between BPEI₆₀₀ and P. aeruginosa LPS. The raw thermogram data obtained when BPEI₆₀₀ was titrated into LPS (FIG. 40). The peaks resulting from each injection were exothermic and gradually became smaller suggesting that the LPS became increasingly saturated with BPEI₆₀₀. These titration data are converted to an isotherm (FIG. 40). The negative ΔH values indicate exothermic binding. This binding profile indicates that there was an interaction between BPEI₆₀₀ and LPS which is likely through electrostatic interactions between cationic amines of BPEI₆₀₀ and anionic phosphates and carboxylate groups of LPS molecules. The x-axis of the thermogram is used to reveal the molar ratio of each species if their respective molecular weights are known. Using an LPS molecular weight of 20 kDa, the molar ratio of ˜2.5 indicates the several molecules of BPEI₆₀₀ can bind to a single LPS molecule. In the bacterial outer membrane environment, this would allow for multiple BPEI₆₀₀ binding events with the inner-core, outer-core, O-antigen, and lipid A regions. The inner-core and outer-core polysaccharides of LPS contain phosphate and carboxylate groups that attract metal ions. These binding sites, and the corresponding metal ions, are located 1-2 nm away from the membrane. Likewise, the LPS O-antigen region contains carboxylates that bind metal ions. The metal ions form bridges between adjacent LPS molecules and this network presents a barrier to the passive diffusion of hydrophilic compounds, including β-lactam antibiotics. However, the ITC data show that cationic BPEI₆₀₀ binds with these anionic sites of LPS. This would cause localized disruption of the LPS-metal network and creates new avenues of access for β-lactams to reach porin transporters imbedded in the membrane lipid tails. In this manner, BPEI₆₀₀ increases intracellular concentration of β-lactam antibiotics but does not need to cross the membrane itself to be effective (FIG. 41).

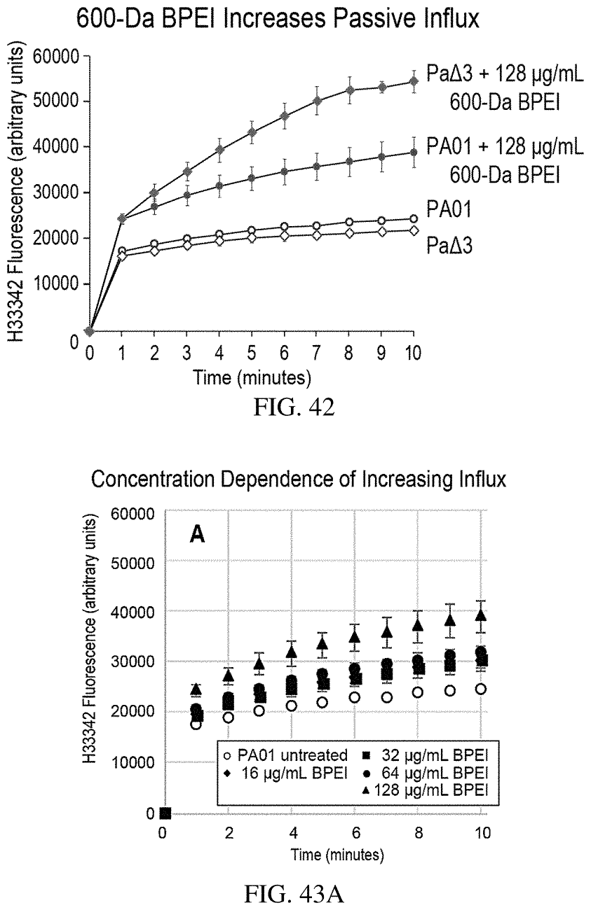

Although BPEI₆₀₀ may be increasing antibiotic influx, it may also be hindering efflux pumps. This can be tested with a fluorescence assay. Using a P. aeruginosa PA01 strain that is multidrug-resistant, bacterial cells were exposed to the fluorescent probe H33342 that is also a substrate for efflux pumps. Fluorescence spectroscopy data measure its accumulation within the cells (FIG. 42). The fluorescence intensity of H33342 is significantly enhanced when bound to the cell membranes and bacterial DNA, levelling off at the maximum intracellular cellular concentration of H33342. The addition of BPEI₆₀₀ increased its fluorescence intensity four-fold. The increase of H33342 intracellular concentration suggests that BPEI₆₀₀ either enhanced the passive diffusion or inactivated the active efflux system. Using this strain's efflux-deficient mutant, PaΔ3, the fluorescence intensity increases further. This shows that BPEI₆₀₀ is not blocking efflux processes. If BPEI was blocking efflux, the intensities would be same because the efflux pump target is absent in PaΔ3 cells and BPEI₆₀₀ would not influence the intracellular concentration in this mutant strain. However, the probe concentration does increase in the presence of BPEI₆₀₀ and thus the effect is attributed to increased drug influx that allows Pseudomonas cells take up more of the fluorescent molecule.

A noteworthy consideration is that the concentration of BPEI (128 μg/mL) used in fluorescence assays is higher than those needed for potentiation or MICs because the cell density needed for a detectable fluorescence signal was much higher. All fluorescence studies used a cell density of ˜6×10⁹ CFU/mL, while checkerboard assays only inoculated a cell density of 5×10⁵ CFU/mL. Therefore, an amount of 128 μg/mL BPEI for fluorescence assays is considered sub-lethal, which is tested and confirmed by resazurin assays. The reduction of resazurin to resorufin occurs via cellular metabolism and thus is an excellent reporter of cell viability. 128 μg/mL of BPEI₆₀₀ for this large cell density (6×10⁹ CFU/mL) is not lethal but causes a 12.5% reduction in cell viability. However, resazurin fluorescence values for cells treated with polymyxin-B are near background levels indicating that these cells are dead. These results have several important impacts. First, the cells in the fluorescence assays are viable and thus drug influx and efflux processes control the intracellular concentration rather than widespread disruption of outer membrane that leads to cell lysis. Secondly, BPEI₆₀₀ is less toxic to P. aeruginosa BAA-47 cells than polymyxin B that is also toxic toward eukaryotic cells. The biocompatibility of BPEI₆₀₀ has been demonstrated against mouse fibroblast cells, immortal human cell lines, and primary human kidney epithelial cells. Finally, at sub-lethal concentration, BPEI₆₀₀ is not disrupting cellular energy metabolism because resazurin reduction occurs via the conversion of NADH/H⁺ to NAD⁺ and thus outer membrane energetics are also likely to be unaffected.

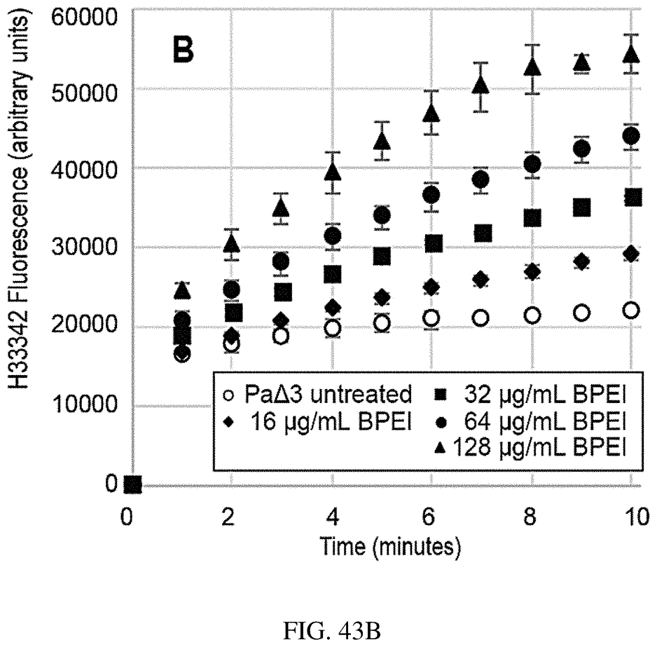

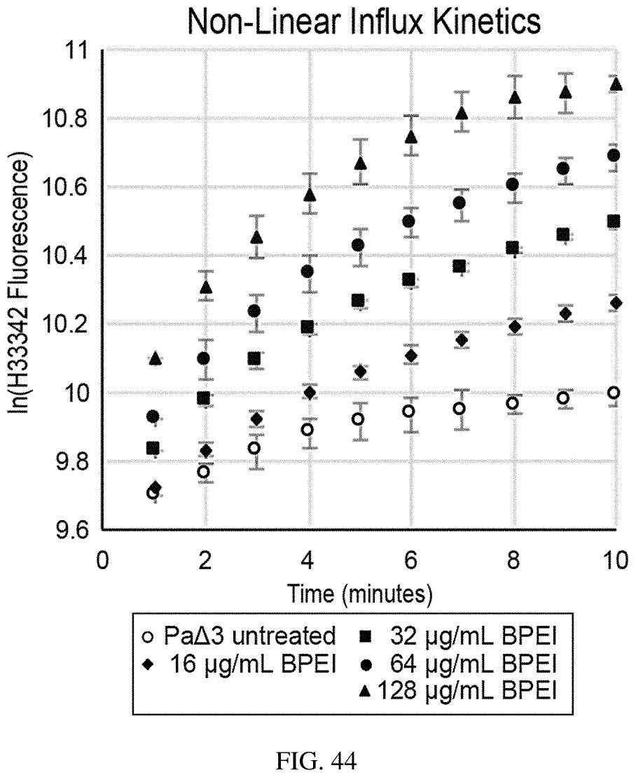

The ability of BPEI₆₀₀ to increase H33342 influx is concentration dependent (FIGS. 43A-43B). In the PAO1 cells, the competition between influx and efflux results in gradual increase in H33342 concentration over time. The data points for H33342 concentration in cells treated with 16 μg/mL and 32 μg/mL of BPEI₆₀₀ overlap whereas the data for 64 μg/mL BPEI₆₀₀ is slightly higher and 128 μg/mL BPEI₆₀₀ gives the highest reading (FIG. 43A). These concentrations are not lethal towards a high density of P. aeruginosa cells. However, the presence of efflux creates a multi-factor condition that complicates the interpretation of biochemical mechanisms. Thus, this experiment was repeated with the efflux-deficient mutant PaΔ3 (FIG. 43B). Inspection of the data reveals that the increase in H33342 concentration over time is not linear but rather exponential in nature, in agreement with a recent kinetic analysis. By plotting the ln [H33342] versus time, it is apparent that the rate of influx is slowest with the lowest concentration of BPEI₆₀₀ (FIG. 44). Thus, a faster rate of influx at higher concentration of BPEI₆₀₀ is due to binding with additional anionic sites of the O-antigen, outer-core, and inner-core regions. Low concentrations of BPEI₆₀₀ limit binding to the outermost regions of LPS. As the BPEI concentration is increased, additional binding sites are occupied until, at levels approaching the MIC, BPEI₆₀₀ binds to lipid A. This scenario allows sub-MIC concentrations of BPEI₆₀₀ to open the LPS network and facilitate diffusion of the H33342 dye. This model also explains the high MIC of BPEI₆₀₀ (16-64 μg/mL), whereas only a fraction of this amount is required for β-lactam potentiation (Table 12). The increased piperacillin MIC in the clinical isolates may be due to overexpression and/or novel β-lactamases, deletion or reduced expression of specific porins, mutations within the porin channel that hinder β-lactam transport, or efflux pumps that are overexpressed. Regardless, BPEI₆₀₀ can restore piperacillin susceptibility in the clinical isolates, causing a considerable reduction of piperacillin MICs.

With regards to other antibiotics, such as meropenem and erythromycin, the situation is more complicated. For the clinical isolates OU15, OU19, and OU22, the meropenem MIC was 16-64 μg/mL but there was no synergy with BPEI₆₀₀, only additivity that caused a modest reduction in MIC values (data not shown). Likewise, the erythromycin MIC's were 256 μg/mL for these 3 isolates and BPEI₆₀₀ exhibited synergy but only reduced the erythromycin MIC by a factor of 4 (data not shown). Recognizing that BPEI₆₀₀ increases the influx of H33342 in a concentration dependent manner, and that the rate of influx also increases with concentration, it is possible to understand the antibiotic potentiation data. Adding BPEI₆₀₀ at ¼^{th} of its MIC value, the diffusion barrier of LPS is reduced. This reduces the piperacillin MIC from over 256 μg/ml to 1-4 μg/mL. The potentiation effect on meropenem is lower, which may be due to hindered porin transport. For erythromycin, reducing the MIC from 256 to 64 μg/mL occurs in the presence of 16 μg/mL of BPEI₆₀₀. As with H33342, erythromycin crosses membranes by passive diffusion and thus, at 16 μg/mL, BPEI₆₀₀ is reducing diffusion barriers from LPS rather than disrupting the membrane itself. Membrane disruption occurs at the MIC of BPEI, 64 μg/mL.

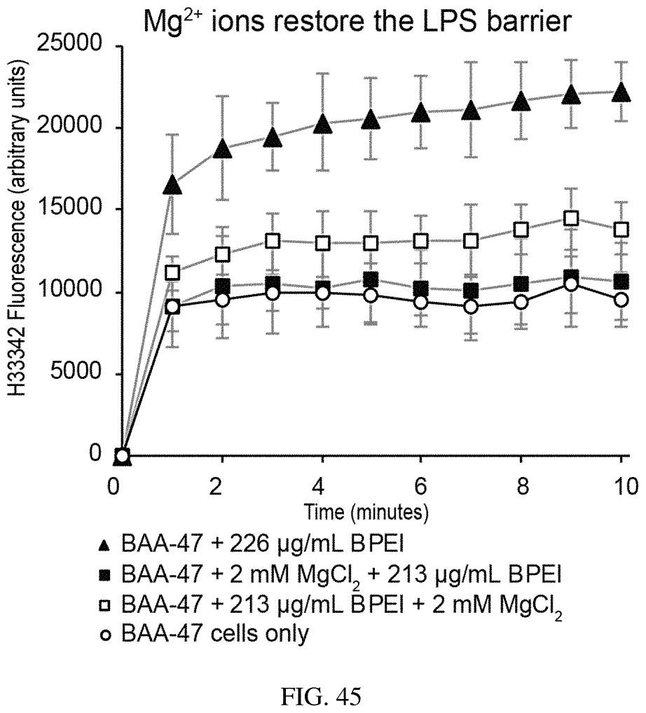

The ability of BPEI₆₀₀ to potentiate β-lactams occurs through electrostatic interactions with LPS anionic sites that also attract metal ions. In the absence of metal ions, the anionic LPS molecules would repel each other and disperse into the extracellular milieu. Instead, Mg²⁺ ions allow the formation of a stable membrane layer by binding to phosphate groups of the lipid A moiety and forming electrostatic bridges between adjacent LPS molecules. Additional phosphate and carboxylate groups are found on heptose and ketodeoxyoctulosonate units of the core oligosaccharides. The O-antigen groups are decorated with hydroxyls and the occasional carboxylate group that can attract metal ions. These anionic LPS sites are critical resistance mechanisms. The primary amines on BPEI₆₀₀ enable it to bind with phosphate and carboxylate groups, and its flexible branches facilitate structural reorganization to reach multiple binding sites within the inner- and outer-core regions of LPS, and span adjacent LPS molecules. The ability of BPEI₆₀₀ to influence membrane permeability, such as the influx of H33342 (FIG. 42), readily occurs at sub-lethal concentrations (data not shown). However, there is a dependence on divalent metal ions. BPEI₆₀₀ weakens the LPS network that otherwise hinders drug uptake. Conversely, adding Mg²⁺ and Ca²⁺ ions stabilize the LPS to strengthen the barrier. This competition is demonstrated in FIG. 45. Here, 2 mM of Mg²⁺ ions were exposed to BAA-47 cells that were treated with BPEI₆₀₀. The lower fluorescence of H33342 indicates that its influx has been slowed by metal ions that reverse weakening of the LPS diffusion barrier, but this process is insufficient to completely dislodge BPEI₆₀₀ from the phosphate/carboxylate binding sites and restore the LPS barrier to its full strength. Similar effects are observed for Ca²⁺ ions. For both metal ions, the order of addition affects the data. When the cells are treated with BPEI₆₀₀ first and metal ions second, the H33342 intensity reaches a magnitude lower than the BPEI-only data but greater than that for cells only (open squares, FIG. 45). However, if the metal ions are added first, the addition of BPEI₆₀₀ does not affect the H33342 intensity (closed squares, FIG. 45). One possible explanation is that the anionic sites of LPS are fully occupied by metals and thus BPEI₆₀₀ cannot bind and promote the diffusion of the fluorescent dye (data not shown). The mechanism for BPEI₆₀₀ is different than other agents that weaken the LPS barrier by chelating metal ions, such as ethylenediamine tetraacetic acid (EDTA). Using data collected with ITC, BPEI₆₀₀ will chelate Cu²⁺ ions but there is no interaction with Mg²⁺ or Ca²⁺ ions (data not shown).

The importance of metal ions in antibiotic mechanism is well established and thus the Clinical and Laboratory Standards Institute (CLSI) guidelines for MIC testing specify the use of cationic-adjusted MHB (CAMHB). These protocols were followed for the growth assay experiments and thus, under standard conditions, metal ions in CAMHB do not interfere with potentiation by BPEI₆₀₀. These data suggest that when cells are grown in CAMHB, the array of metal binding sites within the outer membrane LPS are not fully occupied. This provides an opportunity for BPEI₆₀₀ to establish its own electrostatic interactions with the LPS and create new avenues of access for drugs to reach the membrane. This effect is concentration dependent. Lower amounts of BPEI₆₀₀ facilitate the uptake of piperacillin, a 517 g/mol β-lactam antibiotic that is readily transported to the cytoplasm by transmembrane porins. However, larger amounts of BPEI₆₀₀ are needed to create the larger avenues of access for erythromycin, a 734 g/mol macrolide that reaches the cytoplasm via passive diffusion.

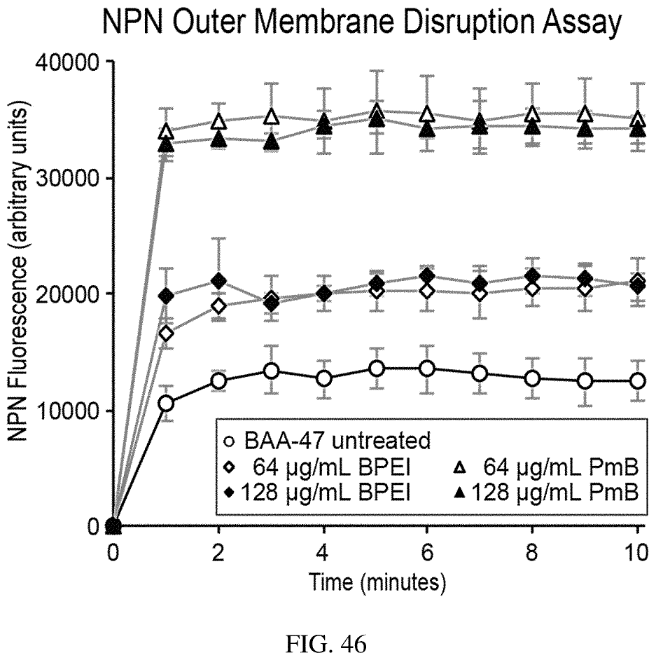

In addition to weakening the LPS barrier to diffusion, BPEI₆₀₀ could be increasing drug influx by changing permeation properties of the outer membrane and perhaps even disrupting the outer membrane lipid bilayer itself. The phenomenon can be tested with the fluorescence probe molecule 1-N-phenylnaphthylamine (NPN) that localizes to the lipid membrane and fluoresces when bound to phospholipids. In the absence of agents that disrupt cell membrane, fluorescence is weak from barriers to passive diffusion. However, then the outer membrane is breached, NPN can easily reach phospholipids of the inner leaflet and fluorescence intensity increases. As shown in FIG. 46, NPN fluorescence reaches value of about 13000 units in a sample of ˜6×10⁹ CFU/ml P. aeruginosa BAA-47 cells. Treating a similar sample with 64 μg/mL of polymyxin-B causes a 2.7 fold increase in fluorescence intensity, which occurs via insertion of polymyxin-B into the membrane via self-promoted uptake. However, 64 and 128 μg/mL of BPEI₆₀₀ cause a 1.5 fold increase in NPN fluorescence and we know that these concentrations of BPEI₆₀₀ are non-lethal. Thus, we suggest that BPEI₆₀₀ is weakening the LPS diffusion barrier, but it is not intercalating into the membrane bilayer that otherwise would lead to a higher increase in NPN fluorescence intensity.

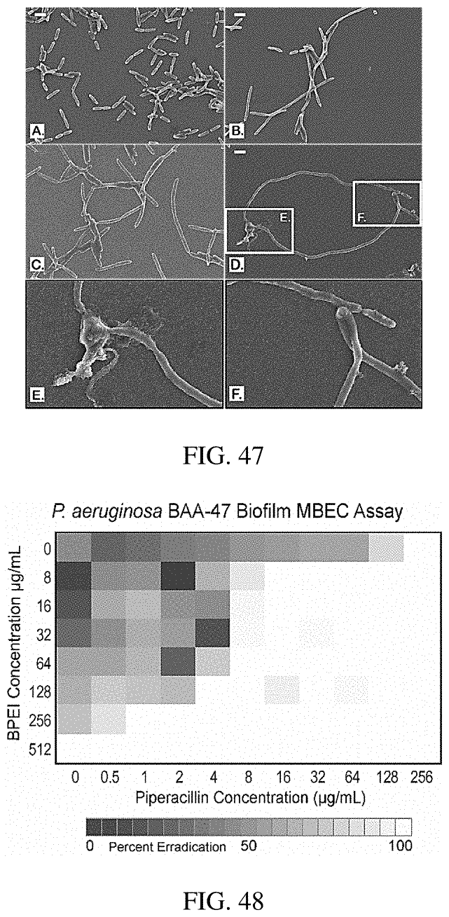

The ability of BPEI₆₀₀ to weaken the LPS diffusion barrier without causing widespread membrane disruption and cell lysis is shown with scanning electron microscopy (SEM). SEM was conducted to examine morphology and the possible effects of BPEI₆₀₀ on bacterial cell division. P. aeruginosa BAA-47 cells were grown to mid-log phase and subjected to four separate treatments: untreated control, sublethal BPEI₆₀₀, sub-lethal piperacillin, and combination of BPEI₆₀₀ and piperacillin, each at sub-lethal combinations. SEM images of the untreated control sample (FIG. 47(A)) show that all the cells have regular rod-shape with a normal size distribution and division septa are clear. BPEI treated cells (FIG. 47(B) are longer and cell-division septa show a gradual narrowing rather than a sharper interface. The piperacillin treated cells (FIG. 47(C)) are longer, do have signs of a well-form division septum, and exhibit signs of cell wall weakening without bursting. Combination of BPEI+piperacillin caused the treated cells (FIG. 47(D)) to rupture (FIG. 47(E)) and show extreme distortions in shape (FIG. 47(F)). The extreme distortions both in size (˜20 times longer than untreated control cells) and shape without obvious division septa suggests that the recruiting, activity, and/or competence of bacterial divisome components is hindered. These cellular morphological changes aid in explaining the killing properties of the BPEI+piperacillin combination although the concentration of each compound is sub-lethal on their own.

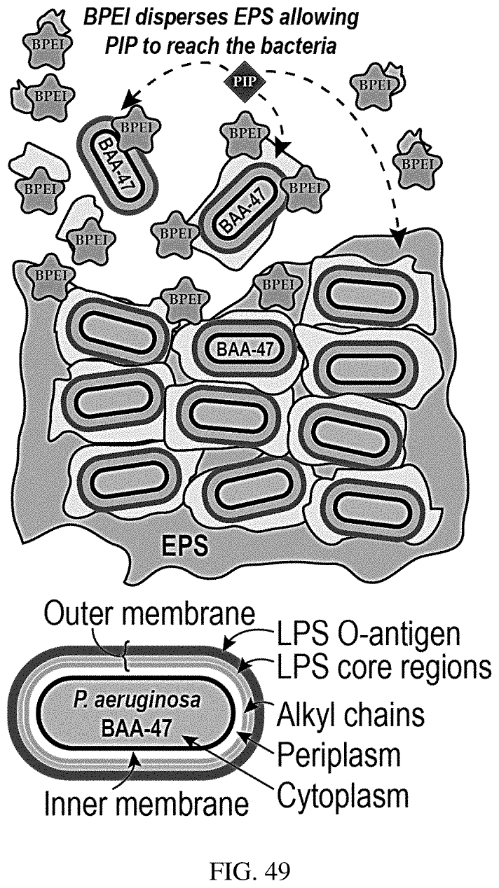

Biofilms are accumulations of microorganisms embedded in a polysaccharide matrix known as extracellular polymeric substance (EPS), which protects the bacteria from antimicrobial agents. Current in-patient treatments include cleansing the wound, debridement, maintaining a moist tissue environment, and—when possible—eliminating the underlying factors that contributed to poor wound healing. BPEI confronts the biofilm directly by disrupting the protective EPS. As shown in FIG. 48, biofilms of P. aeruginosa BAA-47 create additional barriers that require 256 μg/mL of piperacillin to kill the bacteria. This MBEC is significantly higher than the MIC of 4 μg/mL. Likewise, the MBEC of BPEI₆₀₀ is 512 μg/mL, compared to its MIC of 32 μg/mL. A combination treatment results in biofilm eradication with 16 μg/mL of BPEI and 8 μg/mL of piperacillin. As with the planktonic checkerboard assays, this data was collected without a β-lactamase inhibitor. The mechanism of action for disrupting the biofilm relies on the ability of cationic BPEI₆₀₀ to interact with anionic targets. Instead of binding with LPS in the planktonic cells, the biofilm targets are extracellular DNA, anionic polysaccharide Psl, and anionic polysaccharide alginic acid. The presence of the cationic polysaccharide Pel in P. aeruginosa biofilms would repel BPEI, but this affect does not prevent BPEI₆₀₀ from disrupting the biofilm matrix and thus piperacillin can access to the underlying cells (FIG. 49). The data in FIG. 48 also confirms the paradigm that antibiotics effective against planktonic P. aeruginosa are nearly inert against biofilms and resistant strains. When BPEI₆₀₀ binds to EPS, the biofilm disperses because the intermolecular network of exopolymers, protein, and metals ions is disrupted. As a result, quiescent bacteria are released into solution where they become metabolically active and thus the antibiotic can kill the bacteria after additional BPEI molecules reduce LPS barriers to drug influx.

BPEI₆₀₀ has low toxicity, is non-mutagenic, and has high biocompatibility and a low likelihood of causing red blood cell hemolysis. The potentiation of antibiotics varied across different classes, with modest potentiation of β-lactams and no potentiation of erythromycin. Using the efflux deficient mutant PaΔ3, our data show that BPEI₆₀₀ does not inhibit efflux pumps but instead functions by increasing antibiotic permeation. Synthetic diamines are reported to have antibacterial and anti-biofilm properties against P. aeruginosa PAO1 via membrane depolarization and disruption.

Generally regarded as safe and effective, β-lactams are the No. 1 option for treating infections. β-lactams are favored as antibacterial agents. Clinicians prescribed 118 million courses of β-lactam antibiotics in 2011. The recommended treatment of pediatric infections is amoxicillin. These options disappear for infections resistant to β-lactams, which arise from the presence of β-lactamase enzymes that function via hydrolytic cleavage of the lactam ring. Combinations of β-lactams with β-lactamase inhibitors (amoxicillin+clavulanic acid or piperacillin+tazobactam) are used against Gram-negative bacteria. Here, BPEI₆₀₀ targets LPS-mediated resistance in MDR-PA and restores piperacillin efficacy without the need for β-lactamase inhibitors. Additionally, BPEI₆₀₀ will be attracted to anionic components of the bacterial biofilm, resulting in disruption of the extracellular matrix that dissolves the biofilms to enable anti-biofilm activity of piperacillin. Thus, BPEI₆₀₀ may improve patient care outcomes by restoring potency to existing antibiotics with a single potentiator. An advantage of is that it does not need to cross the membrane itself to be effective. By targeting the anionic inner-core and outer-core polysaccharides and biofilm EPS, BPEI₆₀₀ creates new avenues of access for antibiotics to reach their targets. Thus, BPEI does not have to traverse the membrane for potentiation. This contrasts with other cationic antimicrobial agents, such as cationic peptides, aminoglycosides, and polymyxins, whose hydrophobic properties are required for membrane disruption. The delicate balance between potentiation at low concentration and antimicrobial properties are high concentration are possible because we are using BPEI₆₀₀, in contrast to PEIs over 10 kDa that cause widespread membrane disruption and do not have potentiation properties. Nevertheless, the data support the premise that BPEI₆₀₀ increases the influx of fluorescence dyes (FIG. 43A) and does not block efflux pumps (FIG. 43B). There is a strong correlation between increased dye uptake and BPEI₆₀₀ concentration. The presence of efflux pump processes in the WT strain prevents a clear delineation of the trends. However, using the data for the efflux-pump deficient mutant (FIG. 43B) the trend is clear. 64 μg/mL of BPEI₆₀₀ (circles in FIG. 43B) cause and increase in H33342 intensity. However, this amount has a negligible effect of cell viability, as determined from the resazurin assay (Data not shown). This is in stark contrast to polymyxin-B that is lethal to the cells at 64 μg/mL. Thus, we believe that is appropriate to discuss the BPEI₆₀₀ MOA—reducing drug diffusion barriers from LPS—as different than the well-established membrane disruption MOA of polymyxin-B. This interpretation is also supported with the NPN assay data in FIG. 46.

Therefore, in at least certain embodiments, the present disclosure is directed to the compositions, kits, devices, and methods described below.

Clause 1. A method of treating a surface having a biofilm thereon, comprising: conjointly administering to the surface a β-lactam antibiotic, and a potentiating compound comprising a branched poly(ethylenimine) (BPEI) molecule.

Clause 2. The method of clause 1, wherein the BPEI molecule is conjugated to a polyethylene glycol (PEG) molecule forming a PEG-BPEI conjugate.

Clause 3. The method of clause 2, wherein the PEG molecule has an average Mw in a range of about 0.2 kDa to about 5.0 kDa.

Clause 4. The method of clause 1, wherein the surface having the biofilm is a surface of a medical device.

Clause 5. The method of clause 4, wherein the medical device is selected from the group consisting of catheters, cardiovascular devices, orthopedic devices, implants, and tubes.

Clause 6. The method of clause 5, wherein the catheter is selected from the group consisting of intravascular catheters, endovascular catheters, peritoneal dialysis catheters, urethral catheters, peripherally-inserted central catheter (PICC) lines, catheter access ports, and shunts.

Clause 7. The method of clause 5, wherein the medical device is a cardiovascular device selected from the group consisting of heart valves, stents, defibrillators, heart ventricular assist devices, pacemakers, and pacemaker wire leads.

Clause 8. The method of clause 5, wherein the medical device is an orthopedic device selected from the group consisting of orthopedic implants, knee joint replacements, hip joint replacements, shoulder joint replacements, prostheses, spinal disc replacements, orthopedic pins, bone plates, bones screws, and bone rods.

Clause 9. The method of clause 5, wherein the medical device is an implant selected from the group consisting of synthetic bone grafts, bone cements, biosynthetic substitute skins, vascular grafts, surgical hernia meshes, embolic filters, ureter renal biliary stents, urethral slings, gastric bypass balloons, gastric pacemakers, nerve stimulating leads, insulin pumps, neurostimulators, penile implants, silicone implants, saline implants, intrauterine contraceptive devices, cochlear implants, dental implants, dental prosthetics, voice restoration devices, ophthalmic implants, and contact lenses.

Clause 10. The method of clause 5, wherein the medical device is a tube selected from the group consisting of breathing tubes, feeding tubes, intubating tubes, tracheotomy tubes, endotracheal tubes, nasogastric feeding tubes, and gastric feeding tubes.

Clause 11. The method of clause 1, wherein the surface having the biofilm is a tissue surface of a subject.

Clause 12. The method of clause 11, wherein the tissue surface having the biofilm is selected from the group consisting of epithelial surfaces, endothelial surfaces, acute wounds, and chronic wounds.

Clause 13. The method of any one of clauses 1-12, wherein the β-lactam antibiotic, and the potentiating compound are provided in a composition comprising a carrier or vehicle selected from the group consisting of ointments, creams, pastes, gums, lotions, gels, foams, emulsions, suspensions, aqueous solutions, powders, lyophilized powders, solutions, granules, foams, drops, eye drops, adhesives, sutures, aerosols, sprays, sticks, soaps, bars of soap, balms, body washes, rinses, tinctures, gel beads, gauzes, wound dressings, bandages, cloths, towelettes, stents, and sponges.

Clause 14. The method of any one of clauses 1-13 wherein the β-lactam antibiotic is selected from the group consisting of penams, cephems, carbapenems and penems, and monobactams.

Clause 15. The method of any one of clauses 1-14 wherein the BPEI molecule has an average Mw in a range of about 0.1 kilodalton (kDa) to about 25 kDa.

Clause 16. The method of any one of clauses 1-15, wherein the biofilm comprises a bacterium.

Clause 17. The method of clause 16, wherein the bacterium is selected from the group consisting of methicillin-resistant Staphylococcus aureus (MRSA), Enterococcus faecalis, Enterococcus faecium, Staphylococcus aureus, oxacillin-resistant Staphylococcus aureus (ORSA), vancomycin-resistant Staphylococcus aureus (VRSA), a Streptococcus pneumonia, Streptococcus mutans, Streptococcus sanguinis, Staphylococcus epidermidis, methicillin-resistant Staphylococcus epidermidis (MRSE), Bacillus anthracis, Bacillus cereus, Clostridium botulinum, Clostridium botulinum, Listeria monocytogenes, Klebsiella pneumoniae, Streptococcus viridans, Escherichia coli, Pseudomonas aeruginosa, and multi-drug resistant Pseudomonas aeruginosa.

Clause 18. The method of clause 16 or 17, wherein the β-lactam antibiotic and the potentiating compound are conjointly administered to the biofilm have synergistic activity against the bacterium.

Clause 19. The method of clause 18, wherein the β-lactam antibiotic and the potentiating compound together have a synergistic fractional inhibitory concentration (FIC) against the bacterium of the biofilm, wherein the FIC 0.5.

Clause 20. The method of any one of clauses 16-19, wherein the β-lactam antibiotic has a minimum inhibitory concentration (MIC) for the bacterium which is greater than the breakpoint for that bacterium, such that the bacterium is classified as resistant to the β-lactam antibiotic.

Clause 21. An antibiotic composition, comprising: a β-lactam antibiotic, and a potentiating compound comprising a branched poly(ethylenimine) (BPEI) molecule conjugated to a polyethylene glycol (PEG) molecule forming a PEG-BPEI conjugate, wherein the β-lactam antibiotic and the potentiating compound have synergistic activity against a bacterium when administered conjointly.

Clause 22. The antibiotic composition of clause 21, wherein the bacterium against which the antibiotic composition has synergistic activity is selected from the group consisting of methicillin-resistant Staphylococcus aureus (MRSA), Enterococcus faecalis, Enterococcus faecium, Staphylococcus aureus, oxacillin-resistant Staphylococcus aureus (ORSA), vancomycin-resistant Staphylococcus aureus (VRSA), a Streptococcus pneumonia, Streptococcus mutans, Streptococcus sanguinis, Staphylococcus epidermidis, methicillin-resistant Staphylococcus epidermidis (MRSE), Bacillus anthracis, Bacillus cereus, Clostridium botulinum, Clostridium botulinum, Listeria monocytogenes, Klebsiella pneumoniae, Streptococcus viridans, Escherichia coli, Pseudomonas aeruginosa, and multi-drug resistant Pseudomonas aeruginosa.

Clause 23. The antibiotic composition of clause 21 or 22, wherein the β-lactam antibiotic is selected from the group consisting of penams, cephems, carbapenems and penems, and monobactams.

Clause 24. The antibiotic composition of any one of clauses 21-23, wherein the antibiotic composition has a synergistic fractional inhibitory concentration (FIC) against the bacterium, wherein the FIC 0.5.

Clause 25. The antibiotic composition of any one of clauses 21-24, wherein the β-lactam antibiotic has a minimum inhibitory concentration (MIC) for the bacterium which is greater than the breakpoint for that bacterium, such that the bacterium is classified as resistant to the β-lactam antibiotic.

Clause 26. The antibiotic composition of any one of clauses 21-25, wherein the BPEI molecule has an average Mw in a range of about 0.1 kilodalton (kDa) to about 25 kDa.

Clause 27. The antibiotic composition of any one of clauses 21-26, wherein the PEG molecule has an average Mw in a range of about 0.2 kDa to about 5.0 kDa.

Clause 28. The antibiotic composition of any one of clauses 21-27, wherein the antibiotic composition is disposed in a carrier or vehicle.

Clause 29. The antibiotic composition of clause 28, wherein the carrier or vehicle is selected from the group consisting of ointments, creams, pastes, gums, lotions, gels, foams, emulsions, suspensions, aqueous solutions, powders, lyophilized powders, solutions, granules, foams, drops, eye drops, adhesives, sutures, aerosols, sprays, sticks, soaps, bars of soap, balms, body washes, rinses, tinctures, gel beads, gauzes, wound dressings, bandages, cloths, towelettes, stents, and sponges.

Clause 30. A kit, comprising a first container which contains a β-lactam antibiotic, and a second container which contains a potentiating compound comprising a branched poly(ethylenimine) (BPEI) molecule conjugated to a polyethylene glycol (PEG) molecule forming a PEG-BPEI conjugate, wherein the β-lactam antibiotic and the potentiating compound have synergistic activity against a bacterium when administered conjointly.

Clause 31. A method of treating a bacterial infection in a subject, comprising: conjointly administering to the subject an effective amount of a β-lactam antibiotic, and a potentiating compound comprising a branched poly(ethylenimine) (BPEI) molecule conjugated to a polyethylene glycol (PEG) molecule forming a PEG-BPEI conjugate, wherein when administered conjointly, the β-lactam antibiotic and the potentiating compound have synergistic activity against the bacterium causing the bacterial infection.

Clause 32. The method of clause 31, wherein the BPEI molecule has an average Mw in a range of about 0.1 kilodalton (kDa) to about 25 kDa.

Clause 33. The method of clause 31 or 32, wherein the PEG molecule has an average Mw in a range of about 0.2 kDa to about 5.0 kDa.

Clause 34. The method of any one of clauses 31-33, claim 31, wherein the bacterial infection is caused by a bacterium selected from the group consisting of methicillin-resistant Staphylococcus aureus (MRSA), Enterococcus faecalis, Enterococcus faecium, Staphylococcus aureus, oxacillin-resistant Staphylococcus aureus (ORSA), vancomycin-resistant Staphylococcus aureus (VRSA), a Streptococcus pneumonia, Streptococcus mutans, Streptococcus sanguinis, Staphylococcus epidermidis, methicillin-resistant Staphylococcus epidermidis (MRSE), Bacillus anthracis, Bacillus cereus, Clostridium botulinum, Clostridium botulinum, Listeria monocytogenes, Klebsiella pneumoniae, Streptococcus viridans, Escherichia coli, and Pseudomonas aeruginosa.

Clause 35. The method of any one of clauses 31-34, wherein the β-lactam antibiotic is selected from the group consisting of penams, cephems, carbapenems and penems, and monobactams.

Clause 36. The method of any one of clauses 31-35, wherein the β-lactam antibiotic and the potentiating compound together have a synergistic fractional inhibitory concentration (FIC) against the bacterium, wherein the FIC 0.5.

Clause 37. The method of any one of clauses 31-36, wherein the β-lactam antibiotic has a minimum inhibitory concentration (MIC) for the bacterium which is greater than the breakpoint for that bacterium, such that the bacterium is classified as resistant to the β-lactam antibiotic.

Clause 38. The method of any one of clauses 31-37, wherein the BPEI molecule has a Mw in a range of about 0.1 kilodalton (kDa) to about 25 kDa.

Clause 39. The method of any one of clauses 31-38, wherein the bacterial infection comprises a biofilm on or in a tissue surface and the tissue surface is selected from the group consisting of epithelial surfaces, endothelial surfaces, acute wounds, and chronic wounds.

Clause 40. The method of any one of clauses 31-39, wherein the β-lactam antibiotic, and the potentiating compound are provided in a composition comprising a carrier or vehicle selected from the group consisting of ointments, creams, pastes, gums, lotions, gels, foams, emulsions, suspensions, aqueous solutions, powders, lyophilized powders, solutions, granules, foams, drops, eye drops, adhesives, sutures, aerosols, sprays, sticks, soaps, bars of soap, balms, body washes, rinses, tinctures, gel beads, gauzes, wound dressings, bandages, cloths, towelettes, stents, and sponges.

It will be understood from the foregoing description that various modifications and changes may be made in the various embodiments of the present disclosure without departing from their true spirit. The description provided herein is intended for purposes of illustration only and is not intended to be construed in a limiting sense. Thus, while embodiments of the present disclosure have been described herein so that aspects thereof may be more fully understood and appreciated, it is not intended that the present disclosure be limited to these particular embodiments. On the contrary, it is intended that all alternatives, modifications and equivalents are included within the scope of the inventive concepts as defined herein. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulations and compositions described herein, the methods described herein or in the steps or the sequence of steps of the methods described herein without departing from the spirit and scope of the present disclosure.

What is claimed is:

1. A method of treating a surface having a biofilm thereon, comprising:
conjointly administering to the surface a β-lactam antibiotic, and a potentiating compound comprising a branched poly(ethylenimine) (BPEI) molecule.

2. The method of claim 1, wherein the BPEI molecule is conjugated to a polyethylene glycol (PEG) molecule forming a PEG-BPEI conjugate.

3. The method of claim 1, wherein the surface having the biofilm is a surface of a medical device.

4. The method of claim 3, wherein the medical device is selected from the group consisting of catheters, cardiovascular devices, orthopedic devices, implants, and tubes.

5. The method of claim 1, wherein the surface having the biofilm is a tissue surface of a subject.

6. The method of claim 5, wherein the tissue surface having the biofilm is selected from the group consisting of epithelial surfaces, endothelial surfaces, acute wounds, and chronic wounds.

7. The method of claim 1, wherein the β-lactam antibiotic is selected from the group consisting of penams, cephems, carbapenems and penems, and monobactams.

8. The method of claim 1, wherein the biofilm comprises a bacterium selected from the group consisting of methicillin-resistant Staphylococcus aureus (MRSA), Enterococcus faecalis, Enterococcus faecium, Staphylococcus aureus, oxacillin-resistant Staphylococcus aureus (ORSA), vancomycin-resistant Staphylococcus aureus (VRSA), a Streptococcus pneumonia, Streptococcus mutans, Streptococcus sanguinis, Staphylococcus epidermidis, methicillin-resistant Staphylococcus epidermidis (MRSE), Bacillus anthracis, Bacillus cereus, Clostridium botulinum, Clostridium botulinum, Listeria monocytogenes, Klebsiella pneumoniae, Streptococcus viridans, Escherichia coli, Pseudomonas aeruginosa, and multi-drug resistant Pseudomonas aeruginosa.

9. An antibiotic composition, comprising: a β-lactam antibiotic, and a potentiating compound comprising a branched poly(ethylenimine) (BPEI) molecule conjugated to a polyethylene glycol (PEG) molecule forming a PEG-BPEI conjugate, wherein the β-lactam antibiotic and the potentiating compound have synergistic activity against a bacterium when administered conjointly.

10. The antibiotic composition of claim 9, wherein the bacterium against which the antibiotic composition has synergistic activity is selected from the group consisting of methicillin-resistant Staphylococcus aureus (MRSA), Enterococcus faecalis, Enterococcus faecium, Staphylococcus aureus, oxacillin-resistant Staphylococcus aureus (ORSA), vancomycin-resistant Staphylococcus aureus (VRSA), a Streptococcus pneumonia, Streptococcus mutans, Streptococcus sanguinis, Staphylococcus epidermidis, methicillin-resistant Staphylococcus epidermidis (MRSE), Bacillus anthracis, Bacillus cereus, Clostridium botulinum, Clostridium botulinum, Listeria monocytogenes, Klebsiella pneumoniae, Streptococcus viridans, Escherichia coli, Pseudomonas aeruginosa, and multi-drug resistant Pseudomonas aeruginosa.

11. The antibiotic composition of claim 9, wherein the β-lactam antibiotic is selected from the group consisting of penams, cephems, carbapenems and penems, and monobactams.

12. The antibiotic composition of claim 9, wherein the antibiotic composition has a synergistic fractional inhibitory concentration (FIC) against the bacterium, wherein the FIC 0.5.

13. The antibiotic composition of claim 9, wherein the BPEI molecule has an average Mw in a range of about 0.1 kilodalton (kDa) to about 25 kDa.

14. The antibiotic composition of claim 9, wherein the PEG molecule has an average Mw in a range of about 0.2 kDa to about 5.0 kDa.

15. The antibiotic composition of claim 9, comprising an antibiotic/BPEI mass ratio in a range of 100:1 to 1:100.

16. A method of treating a bacterial infection in a subject, comprising:
conjointly administering to the subject an effective amount of a β-lactam antibiotic, and a potentiating compound comprising a branched poly(ethylenimine) (BPEI) molecule conjugated to a polyethylene glycol (PEG) molecule forming a PEG-BPEI conjugate, wherein when administered conjointly, the β-lactam antibiotic and the potentiating compound have synergistic activity against the bacterium causing the bacterial infection.

17. The method of claim 16, wherein the bacterial infection is caused by a bacterium selected from the group consisting of methicillin-resistant Staphylococcus aureus (MRSA), Enterococcus faecalis, Enterococcus faecium, Staphylococcus aureus, oxacillin-resistant Staphylococcus aureus (ORSA), vancomycin-resistant Staphylococcus aureus (VRSA), a Streptococcus pneumonia, Streptococcus mutans, Streptococcus sanguinis, Staphylococcus epidermidis, methicillin-resistant Staphylococcus epidermidis (MRSE), Bacillus anthracis, Bacillus cereus, Clostridium botulinum, Clostridium botulinum, Listeria monocytogenes, Klebsiella pneumoniae, Streptococcus viridans, Escherichia coli, Pseudomonas aeruginosa, and multi-drug resistant Pseudomonas aeruginosa.

18. The method of claim 16, wherein the β-lactam antibiotic is selected from the group consisting of penams, cephems, carbapenems and penems, and monobactams.

19. The method of claim 16, wherein the β-lactam antibiotic and the potentiating compound together have a synergistic fractional inhibitory concentration (FIC) against the bacterium, wherein the FIC≤0.5.

20. The method of claim 16, wherein the β-lactam antibiotic has a minimum inhibitory concentration (MIC) for the bacterium which is greater than the breakpoint for that bacterium, such that the bacterium is classified as resistant to the β-lactam antibiotic.
